(19) United States
(12) Patent Application Publication
(10) Pub. No.: US 2026/0176682 A1
(43) Pub. Date: Jun. 25, 2026

(54) COMPUTER-IMPLEMENTED METHOD FOR IDENTIFYING A DNA MODIFICATION

(71) Applicant: UMC UTRECHT HOLDING B.V., Utrecht (NL)

(72) Inventors: Jeroen DE RIDDER, Utrecht (NL); Marc Pagès GALLEGO, Utrecht (NL); Carlo VERMEULEN, Utrecht (NL)

(21) Appl. No.: 19/128,886

(22) PCT Filed: Nov. 7, 2023
(86) PCT No.: PCT/NL2023/050586

§ 371 Date: May 9, 2025

(30) Foreign Application Priority Data
Nov. 11, 2022 (NL) 2033509
Oct. 10, 2023 (NL) 2035998

(51) Int. Cl. C12Q 1/6869

(52) U.S. Cl. CPC C12Q 1/6869; C12Q 2600/154

(57) ABSTRACT

The present invention relates to computer-implemented methods for identifying a DNA modification in DNA sequencing data for the intraoperative classification of a disease and/or disorder, to methods for identifying a disease and/or disorder, to methods of the pre-operative configuring of a DNA modification-based DL-classifier and to methods of treatment and surgery.

### TECHNICAL FIELD

The present invention relates to computer-implemented methods for identifying a DNA modification in DNA sequencing data for the intraoperative classification of a disease and/or disorder, to methods for identifying a disease and/or disorder, and to methods of the pre-operative configuring of a DNA modification-based DL-classifier.

### BACKGROUND

A big challenge in some diseases and/or disorders, for example in the treatment of a tumor, such as a brain tumor, is that taking a biopsy is often not possible, impractical or too invasive. This means that the type and/or stage of the disease and/or disorder, for example the tumor type, is often unknown at the time of surgery, while often a choice must be made for surgical resection, for example between radical and conservative resection. Rapid histological analysis is currently used to determine the surgical strategy. At a later point, a full molecular classification is made, to determine the exact cancer type based on the methylome of the tumor. In a significant number of cases, the molecular diagnosis differs from the histological assessment, and an additional more radical surgery is needed; or the resection was too radical in hindsight, and side-effects of the surgery could have been avoided.

Molecular classification is based on DNA modification patterns, for example DNA methylation patterns. In the past decades vast numbers of patient derived molecular profiles using microarrays or other high-throughput profiling techniques have been generated and coupled to clinical annotations. In routine practice, Illumina Infinium arrays are a widely used tool to discern the molecular classes based on the modification, e.g. methylation, signal in hundreds of thousands of DNA, e.g. CpG, sites. A major drawback is that these arrays take several days to process, and the result is thus only obtained after surgery.

In light of the foregoing, a fast and comprehensive DNA modification-based classification method would be highly desirable. In particular, there is a clear need in the art for DNA modification-based classification methods or computer-implemented methods that can be used to provide a classification of a disease and/or disorder, for example a tumor, during surgery.

### Definitions

For purposes of the present invention, the following terms are defined below.

As used herein, the singular forms “a,” “an” and “the” include plural referents unless the context clearly dictates otherwise. For example, a method for identifying a modification according to the invention includes the identification of a plurality of such modifications (e.g. 10's, 100's, 1000's, 10's of thousands, 100's of thousands, millions, or more modifications). As used herein, the term “and/or” indicates that one or more of the stated cases may occur, alone or in combination with at least one of the stated cases, up to with all of the stated cases.

As used herein, the term “biomarker” refers to a characteristic which can be evaluated and measured as indicators of normal biological processes, pathogenesis or response to therapy. Cancer biomarkers may be diagnostic, prognostic, predictive, or used to monitor treatment responses.

As used herein, the term “comprising” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, the term “DNA methylation” refers to an epigenetic mechanism that may affect gene expression. DNA methylation is formed by the addition of a methyl group to the 5′ position of cytosine residues within CpG sites (regions of DNA where a cytosine nucleotide is followed by a guanine nucleotide) DNA methylation may cover any one or more of promoters, intergenic, intronic and/or exonic regions of the genome. Aberrant DNA methylation has been shown to promote tumor onset, development, progression and recurrence.

As used herein, the term “exemplary” means “serving as an example, instance, or illustration,” and should not be construed as excluding other configurations disclosed herein.

As used herein, “microarray”, for example as used as in the term “hybridization microarray”, refers to a DNA microarray or a similar technique comprising a collection of orderly microscopic DNA spots, called features, each with thousands of identical and specific probes attached to a solid surface, such as glass, plastic or silicon biochip.

Hybridization microarray technology makes use of the property of complementary nucleic acid sequences to specifically pair with each other. Fluorescently labelled target sequences that bind to a probe sequence generate a signal. The total strength of a signal from a spot depends on the amount of target sample binding tot the probes present on that spot. The intensity of a feature is compared to the intensity of the same feature under a different condition (relative quantitation), and the identity of the feature is known by its position.

As used herein, “nanopore sequencing” refers to sequencing based on nanopores or similar sequencing techniques, for example for whole genome sequencing. The principle of operation of the nanopore sequence technique is the analysis of the DNA strand directly as the molecule is drawn through a tiny pore suspended in a membrane. Changes in electrical current, or tunnelling currents, are used to read off the chain of bases. The technology may be portable and may provide sequence data in real time, and that typically contains higher error rates than e.g. Illumina sequencing.

### BRIEF DESCRIPTION OF DRAWINGS

FIG. 1: Procedural steps from a reference dataset from which, by a simulation module, a dataset is obtained that is used in a training module. The inference data is analysed and a class is assigned to the inference data.

FIG. 2: Simulation showing the number/count of reads of a particular length (top) when performing Nanopore sequencing and the accumulated number of reads over time (bottom).

FIG. 3: Data obtained from Illumina methylation arrays, available from GEO (GSE109381). The dataset consists of methylation profiles from 2801 samples from 82 different cancer classes and 9 control tissues. Cancer classes are grouped into 14 larger family group.

FIG. 4: Balanced upsampling of the data obtained from Illumina methylation arrays for all cancer classes (see FIG. 3) to the same number of samples as the most represented class.

FIG. 5: Cross-validation results after 15 minutes of sequencing with 86.5% accuracy when comparing the truth. The y-axis shows the true label of samples while the x-axis shows the label as predicted by the Sturgeon classifier. Greyscale indicates the percentage of predictions for a particular true label class (white=0%, black=100%).

FIG. 6: Cross-validation results after 30 minutes of sequencing with 89.8% accuracy when comparing the truth. The y-axis shows the true label of samples while the x-axis shows the label as predicted by the sturgeon classifier. Greyscale indicates the percentage of predictions for a particular true label class (white=0%, black=100%).

FIG. 7: Cross-validation results after 60 minutes of sequencing with 91.3% accuracy when comparing the truth. The y-axis shows the true label of samples while the x-axis shows the label as predicted by the sturgeon classifier. Greyscale indicates the percentage of predictions for a particular true label class (white=0%, black=100%).

FIG. 8: Cross-validation results as a median and mean F1 score as an accuracy metric of data. The y-axis shows the true label of samples while the x-axis shows the label as predicted by the Sturgeon classifier. Greyscale indicates the percentage of predictions for a particular true label class (white=0%, black=100%).

FIG. 9: Cross-validation results of surgery classified samples after 15 minutes of sequencing with 94.4% accuracy when comparing the truth. The y-axis shows the true label of samples while the x-axis shows the label as predicted by the Sturgeon classifier. Greyscale indicates the percentage of predictions for a particular true label class (white=0%, black=100%).

FIG. 10: Cross-validation results of surgery classified samples after 30 minutes of sequencing with 96.3% accuracy when comparing the truth. The y-axis shows the true label of samples while the x-axis shows the label as predicted by the Sturgeon classifier. Greyscale indicates the percentage of predictions for a particular true label class (white=0%, black=100%).

FIG. 11: Cross-validation results of surgery classified samples after 60 minutes of sequencing with 97.1% accuracy when comparing the truth. The y-axis shows the true label of samples while the x-axis shows the label as predicted by the Sturgeon classifier. Greyscale indicates the percentage of predictions for a particular true label class (white=0%, black=100%).

FIG. 12: Cross-validation results of surgery classified samples as a median and mean F1 score as a classification accuracy metric of data.

FIG. 13: Accuracy increase with sequencing time for the “Diagnostic” classification system versus the more aggregated “surgery grouping” classification system.

FIG. 14: Fraction of correctly predicted samples, per classified sample, based on four models (model 0 to 3) for various tissues.

FIG. 15: Fraction of correctly predicted samples depending on the number of probes for each of the various tissue samples (<20 k probes, 20-40 k probes and >40 k probes).

FIG. 16: Result of several iterations of adaptive upsampling for each of the various tissue samples.

FIG. 17: Adaptive upsampling changes the accuracy performance depending on the class (x-axis) and that it overall improves accuracy significantly (y-axis). The effect is more pronounced at shorter sequencing times.

FIG. 18: Evaluation of performance of the Sturgeon classifier on a hold-out test fold (25% of the reference dataset).

FIG. 19: Prediction (y-axis) of samples having the correct label (lines with circles) or the incorrect label (lines with squares) over time sequenced (x-axis) for various tissue samples

FIG. 20: Classifier score over time. Prediction scores (y-axis) of patient samples (nanopore sequenced samples). The current method was applied to 14 pediatric CNS cancer DNA samples from the Prinses Maxima Centrum Biobank. Lines indicate the confidence score at 5 minute intervals. In each case, the highest scoring class after 15 minutes of sequencing corresponded to the clinical diagnosis.

FIG. 21: Publicly available nanopore sequencing runs from 415 CNS cancer samples were obtained from GEO (GSE 209865). The current method was applied to each sample. Some samples were undersequenced, and in said undersequenced samples, the method did not reach the confidence threshold (0.8). In samples where the 0.8 threshold was reached, accuracy was 100%. Horizontal dashed line indicates 0.8 confidence threshold, vertical lines indicate the range of probes in which the method was trained on.

FIG. 22: Downsampling profiles and upsampling the number of samples. Since only a fraction of the methylation array data is used the sampling can be performed multiple times to upsample the number of “patients” per diagnosis class to create a very large set (>10.000 profiles) of simulated nanopore runs for training and validation.

FIG. 23A is a block diagram illustrating an example architecture of a trained neural network for identifying a type of tumor in a patient by using the trained neural network to process a sparse DNA methylation profile for the patient, in accordance with some embodiments of the technology described herein.

FIG. 23B is a block diagram illustrating a specific example of a trained neural network having the neural network architecture shown in FIG. 23A, in accordance with some embodiments of the technology described herein.

FIG. 24A is a block diagram of a software for: (1) training a machine learning (ML) model to predict the type of a patient's tumor using a sparse DNA methylation profile generated for the patient; and (2) using the trained ML model to do so, in accordance with some embodiments of the technology described herein.

FIG. 24B is a block diagram illustrating intraoperative use of a CNS tumor type prediction system comprising a nanopore sequencing apparatus for sequencing a biological sample from a patient undergoing surgery and one or more computing device(s) to generate, from nanopore sequencing data, a sparse DNA methylation profile and process it by using a trained ML model to output an indication of the type of the patient's tumor, in accordance with some embodiments of the technology described herein.

FIG. 25A is a flowchart of an illustrative process 2500 for predicting the type of a patient's tumor using a sparse DNA methylation profile generated for the patient from DNA sequencing data for the patient, in accordance with some embodiments of the technology described herein.

FIG. 25B is a flowchart of another illustrative process 2550 for predicting, while a patient is undergoing surgery, the type of the patient's tumor using a sparse DNA methylation profile generated from DNA sequencing data for the patient obtained by nanopore sequencing, in accordance with some embodiments of the technology described herein.

FIG. 26 depicts an illustrative implementation of a computer system that may be used in connection with some embodiments of the technology described herein.

FIG. 27A depicts nanopore sequencing runs that were simulated from the Capper et al.¹⁴ reference set comprising 2,801 labelled methylation profiles from CNS tumor and control samples. Sequencing data was simulated based on existing nanopore sequencing runs (read length distribution and throughput); as these simulations produce very sparse samples millions of unique samples can be simulated.

FIG. 27B depicts fourfold cross-validation was performed by rotating the folds to obtain four models that were used in the final prediction of external microarray data and nanopore sequencing data.

FIG. 27C depicts the performance of Sturgeon on the four test folds of the Capper et al. dataset (added up for the four submodels). F1 scores for each reference label at 40 min of simulated sequencing (approximately 97% missing values compared with microarray data). Solid bars indicate the F1 score for the highest-scoring class and transparent bars show the F1 score for the top three highest-scoring classes.

FIG. 28A depicts classification performance over time on nanopore runs simulated from pediatric CNS tumor methylation arrays. Each series of bars corresponds to one of the 68 cases for which a clear Heidelberg classifier result was obtained (Heidelberg score >0.84). Bars indicate at each timepoint the proportion of outcomes when a 0.95 confidence score is used. The correct fraction is colored by class; colors correspond to those in FIG. 27C. Unclear (no class reached a confidence score ≥0.95 or a control class reached a confidence score ≥0.95) classifications are shown in grey, wrong classes with a confidence score ≥0.95 are yellow.

FIG. 28B depicts a schematic similar to FIG. 28A, but for the 26 samples for which the Heidelberg classifier was inconclusive (Heidelberg score <0.84; unclear cases).

FIG. 28C depicts a stacked bar graph to show the effect of different tumor fractions on classifier performance on short sequencing simulations (on average 8,128 CpG sites covered, equivalent to roughly 20 min of sequencing). Nanopore sequencing experiments were simulated from the reference samples from the Capper et al. dataset. The reported sample purity was used as a baseline and control tissue reads were added to simulate lower sample purities. The fraction of correct and incorrect classifications over or under the confidence threshold (≥0.95) are shown, as well as the number of simulations where the classifier predicted the sample as control tissue.

FIG. 28D depicts a schematic similar to FIG. 28C, with a higher sequencing depth (17,943 CpG sites covered on average, equivalent to approximately 40 min of sequencing).

FIG. 29A depicts two representative examples of Sturgeon classification on nanopore-sequenced samples. The x axis indicates the sequencing time (5-min pseudotime intervals) and the y axis indicates the confidence score. Circles indicate the confidence score of the correct class; diamonds indicate the confidence score of incorrect classes (classes with averaged scores lower than 0.1 are omitted). Asterisks indicate the first timepoint at which the confidence score of the correct class was higher than 0.95.

FIG. 29B depicts Sturgeon classification scores for 27 paediatric CNS tumor samples at increasing sequencing time (5-min pseudotime intervals). Only the confidence score of the correct class is plotted; see FIG. 35 for complete results for each sample.

FIG. 29C depicts Sturgeon classification results on the publicly available data from Kuschel et al. (415 sequencing runs). Samples for which the highest-scoring class is correct are indicated as circles and samples for which the highest-scoring class is incorrect are shown as crosses. Points are filled based on the correct class according to the color scheme shown in FIG. 27C.

FIG. 30 depicts intraoperative sequencing turnaround time. Timeline for surgery and intraoperative sample analysis for INTRA_4, with the turnaround time and required time per processing step indicated (in minutes). Circles indicate the confidence score of the correct class; diamonds indicate the confidence score of incorrect classes (classes with averaged confidence scores lower than 0.1 are omitted). The asterisk indicates the first timepoint at which the score of the correct class was higher than 0.95.

FIG. 31A depicts five samples that were run with adaptive sampling on half of the available channels. Box plots indicate the number of 450K array CpG probe sites during sequencing, normalized to the amount of available sequencing channels; minimum and maximum bounds represent the 25th and 75th percentiles, respectively; and the center bound represents the median; whiskers extend to 1.5 times the interquartile range. Dots indicate the underlying data.

FIG. 31B depicts robustness analysis results of sample PMC 68 on adaptive (left) and non-adaptive (right) channels (results for all samples are presented in FIG. 63). Reads were accumulated in resampled orders (n=100) at a rate based on the average MinION sequencing speed. Sturgeon was then applied to all permutations. Colors indicate the type of prediction made by Sturgeon: plain color (correct class and score ≥0.95), color with diagonal lines (correct class and score <0.95), grey with diagonal lines (incorrect class and score <0.95), black (incorrect class and score ≥0.95).

FIG. 32A depicts the Sturgeon performance at 40 min of simulated sequencing. Specifically, a confusion matrix showing the highest scoring class for each reference label at 40 min of simulated sequencing (˜97% missing values from microarray data)

FIG. 32B depicts a confusion matrix and F1 scores at 40 min of simulated sequencing when scores are aggregated on the family level.

FIG. 32C depicts F1 scores at different sequencing depths (represented by the average number of covered 450 K array methylation sites) when classifying by subclass, by the correct subclass being in the top 3 of highest scoring classes and at the family level. Box plot minimum and maximum bounds represent the 25th and 75th percentiles, respectively, and the center bound represents the median. Whiskers extend to 1.5 times the interquartile range.

FIG. 32D depicts a true positive rate for each subclass at 40 min of sequencing at the 0.95 confidence threshold.

FIG. 33A depicts a classification performance over time on nanopore runs simulated from pediatric CNS tumor methylation arrays, specifically, a clear diagnosis group (Heidelberg classifier score >0.84).

FIG. 33B depicts the difficult diagnosis group (Heidelberg classifier score <0.84).

FIG. 33C depicts the distribution of the number of CpG sites covered at each simulated timepoint.

FIG. 34A depicts a histogram showing the reported sample purity in the Capper et al. training dataset.

FIG. 34B depicts that due to the inherent sample purity, the number of samples where high purity can be simulated is limited. This histogram shows the number of used simulations at each purity level.

FIG. 34C and FIG. 34D depict bar plots showing the simulation results at a 0.95 (c) or 0.8 (d) cutoff at different sequencing depths (represented by the average number of 450 K CpG sites covered). Bars are colored by correct and confident (score above cutoff) outcomes, correct but low confidence outcomes (highest scoring class is correct, but the score is below the confidence threshold), high and low confidence control outcomes (the highest scoring class is one of the control classes), and wrong outcomes where an incorrect class scores highest below or above the confidence threshold.

FIG. 35 depicts the retrospective nanopore sequencing results. Sturgeon confidence scores for 27 pediatric CNS tumor samples (duplicates indicated by appended “_1” to the sample name at increasing sequencing time (5 min pseudo time intervals). Top bar indicates the sample name. Circles indicate the predicted score of the correct class; diamonds indicate the predicted score of incorrect classes (classes with overtime averaged scores lower than 0.1 are omitted). Asterisks indicate the first time point where the score of the correct class was higher than 0.95. Horizontal line indicates the 0.95 threshold.

FIG. 36 depicts the robustness analysis results. For each sample sequence reads were randomly sampled to reflect a nanopore run at a specific duration. 100 simulations were generated for each timepoint. Colored bars indicate correct outcomes above the confidence threshold (0.95), dashed colored bars indicate correct outcomes below the threshold, gray dashed bars indicate unclear outcomes and black bars indicate wrong outcomes above the threshold.

FIG. 37A depicts copy number variations. The sample was sequenced to 1.2 million reads, dots represent the normalized coverage (Methods) for 2 Mb bins, red lines indicate the DNAcopy segmentation result which clearly shows the 1p/19q codeletion. Bins that fall within segments with a log 2 value <−0.5 are colored blue and bins that fall in segments >0.5 are colored green.

FIG. 37B and FIG. 37C depict the results of repeated analysis after 50,000 and 20,000 random reads were subsampled; in both sequence depths the 1p deletion is clearly visible, and the 19q deletion is visible but less clearly defined.

FIG. 37D and FIG. 37E depict the segmentation results from 10 random downsamplings at a sequence depth of 20.000 and 50.000 sequence reads respectively red lines indicate the segmentation of the full dataset and blue dashed lines show the result of individual subsamplings.

FIG. 38 depicts intraoperative sequencing results. Sturgeon confidence scores over time for the 25 intraoperative sequencing experiments. Class corresponding to the integrated histomolecular diagnosis are shown in circles (with the exception of INTRA_24, where the highest scoring Heidelberg V11b4 class is indicated as a circle) and other classes are shown as squares. Headers are colored following the same style as described in the circles, with the exception of INTRA_11 (Germinoma, class not in the classifier) and INTRA_13 (exotic case) which are colored white.

FIG. 39 depicts an overview of the intraoperative sequencing cases.

FIGS. 40A-40C show Concordance between nanopore sequencing and Infinium arrays. FIG. 40A shows Illumina Infinium arrays of five samples were binarized using different beta cutoffs (x-axis) and the calls per CpG site were compared to those generated using nanopore sequencing (R9 chemistry, PromethION flowcell, Megalodon methylation calling). FIG. 40B shows the same data was compared using a symmetrical two-sided cutoff (sites with a beta value in-between the cutoffs were discarded). FIG. 40C shows the fraction of interpretable sites when using two sided cutoffs and discarding sites with beta values in-between the cutoffs.

FIG. 41 shows F1 scores for each class at different simulated sequencing depths. Transparent bars indicate performance when taking the top 3 scoring classes into account.

FIG. 42 shows F1 scores on the family level at different sequencing depths.

FIG. 43 shows the expected versus observed True Positive Rate for each different class in the validation fold prior to calibration. Red bars highlight deviation between expected and true TPR. Bottom right plot represents all aggregated classes.

FIG. 44 shows the expected versus expected True Positive Rate for each different class in the validation fold after temperature scaling. Red bars highlight deviation between expected and true TPR. Bottom right plot represents all aggregated classes.

FIG. 45 shows the expected versus expected True Positive Rate for each different class in the test fold prior to calibration. Red bars highlight deviation between expected and true TPR. Bottom right plot represents all aggregated classes.

FIG. 46 shows the expected versus expected True Positive Rate for each different class in the test fold after temperature scaling. Red bars highlight deviation between expected and true TPR. Bottom right plot represents all aggregated classes.

FIG. 47 shows the true Positive Rate for each class when using a cutoff of 0.8. Asterisks indicate samples where the TPR is below 0.8.

FIGS. 48A-48B show a confusion matrix for pediatric samples using a cutoff of 0.95. For each sample 500 nanopore runs were simulated at timepoint 1 and 3. The number of Sturgeon outcomes for each class is indicated in greyscale, unclear outcomes are also listed in the bottom row. Red squares indicate the Heidelberg classifier outcome (if conclusive), the blue cross indicates the clinical diagnosis.

FIGS. 49A-49B show a confusion matrix for pediatric samples using a cutoff of 0.8. For each sample 500 nanopore runs were simulated at timepoint 1 and 3. The number of Sturgeon outcomes for each class is indicated in greyscale, unclear outcomes are also listed in the bottom row. Red squares indicate the Heidelberg classifier outcome (if conclusive), the blue cross indicates the clinical diagnosis.

FIGS. 50A-50B show overlap between the nanoDx pipeline and Sturgeon classification on an external dataset. FIG. 50A shows the y-axis indicates the Sturgeon confidence score for each sample. x-Axis indicates the number of measured (450K array) CpG sites. Samples are colored by the performance of both. Sturgeon and nanoDx. Dark blue indicates that both methods were correct (N=354), light blue indicates that nanoDx was incorrect and Sturgeon was correct (N=29). Orange indicates that nanoDX was correct and Sturgeon was incorrect (N=20), red indicates both methods were incorrect. FIG. 50B shows an Upset plot showing the performance of Sturgeon and nanoDX.

FIG. 51A shows MinION sequencing metrics. Graphs indicate the sequencing speed of MinION devices used in our experiments. Indicated over time in non-cumulative (left) and cumulative (right) bins, are the number of sequenced bases, number of sequenced reads, the number of CpG methylation calls (independent of relevance to 450K arrays) and read length.

FIG. 51B indicates the number of CpG methylation calls versus the number of sequenced bases. Blue colors are R9 chemistry, flowcells and workflow and red hues are R10 chemistry, flowcells and workflow.

FIG. 52 shows the classification results from a retrospective oligodendroglioma case (UMCU_1). Sequencing results in high but <0.95 confidence scores for IDH mutant Astrocytoma (dark yellow) and IDH mutant oligodendroglioma (light yellow).

FIG. 53 shows Copy Number Variation profiles for PMC_60. Sample was sequenced on a nanopore MinION to a depth of ˜350.000 sequence reads. Top graph shows the CNV profile for the full dataset. The second plot shows the segmentations obtained from the full profile in red, blue dashed lines indicate the segments found in 10 independent samplings of 50.000 and 20.000 sequence reads. The bottom plot shows the CNV profile as it was generated from Whole Exome Sequencing.

FIG. 54 shows the copy Number Variation profiles. Copy Number Variations shown for nanopore sequenced samples PMC_29, PMC_69, PMC_53 and PMC_42. Sample was sequenced on a nanopore MinION, depth is shown in the legend. Top graph shows the CNV profile as obtained from Whole Exome Sequencing. The second plot shows the segmentations obtained from the full profile in red, blue dashed lines indicate the segments found in 10 independent samplings of 50.000 sequence reads.

FIG. 55 shows a brainstem classifier confusion matrix and F1 scores. Sturgeon brainStem performance on the four test folds of the Capper et al. dataset. Confusion matrix showing the highest scoring class for each reference label at 40 minutes of simulated sequencing (˜97% missing values from microarray data). Bars on the right side of the plot indicate the top 1 (solid) and top 3 (transparent) F1-scores per reference label class.

FIG. 56 shows confidence over time for the brainstem classifier on brainstem samples. These plots show the confidence of the brainstem classifier with reads accumulated at a rate expected for a MinION run. Asterisks indicate the first timepoint the confidence score is higher than 0.95. Brown colors indicate that the model classifies a sample as “non-brainstem”.

FIG. 57 shows confidence over time for the general classifier on brainstem samples. These plots show the confidence of the general classifier with reads accumulated at a rate expected for a MinION run. Asterisks indicate the first timepoint the classification score is higher than 0.95.

FIG. 58 shows Confidence over time for the brainstem classifier on non-brainstem samples. These plots show the confidence of the brainstem classifier with reads accumulated at a rate expected for a MinION run. Asterisks indicate the first timepoint the classification score is higher than 0.95. Grey samples are from classes not present in the brainstem classifier. Brown colors indicate that the model classifies samples as non-brainstem.

FIG. 59 shows robustness of the brainstem and general classifier for brainstem samples. Reads were randomly sampled for each timepoint 100× and classified by the brainstem and general classifier. Lines indicate the fraction of simulations that reached a >0.95 confidence score for the correct class in the general (orange) and brainstem (green) classifier.

FIG. 60 shows robustness of the brainstem and general classifier for non-brainstem samples. Reads were randomly sampled for each timepoint 100× and classified by the brainstem and general classifier. Lines indicate the fraction of simulations that reached a >0.95 confidence score for the correct class in the general (orange) and brainstem (green) classifier.

FIG. 61 shows read length in adaptive versus non adaptive sampling. For a single sequence experiment with adaptive sampling enabled on half of the channels, the left plot shows the read length distribution for non-adaptive sampling channels. The right plot shows the read length distribution for the adaptive sampling channels, with lengths colored by whether the read was rejected or not.

FIG. 62 shows throughput of adaptive versus non adaptive channels. For both adaptive and non-adaptive channels, the number of sequenced bases was calculated over time. As expected, adaptive channels spend time ejecting reads, and thus lose some throughput. Solid line indicates the mean, the shadowed area indicates the standard deviation (n=5).

FIG. 63 shows robustness of adaptive versus regular sequencing. Five samples were sequenced using adaptive sampling enabled on half of the channels. The top row shows the results of 100 simulations for each of 12 different simulated sequencing times. The fraction of correct classifications is shown in the class color, dashed lines indicate the number of classifications with a score <0.95. Gray dashed lines indicate an incorrect class had the highest confidence score but <0.95, black indicate simulations with a wrong class and confidence >0.95 (N=1).

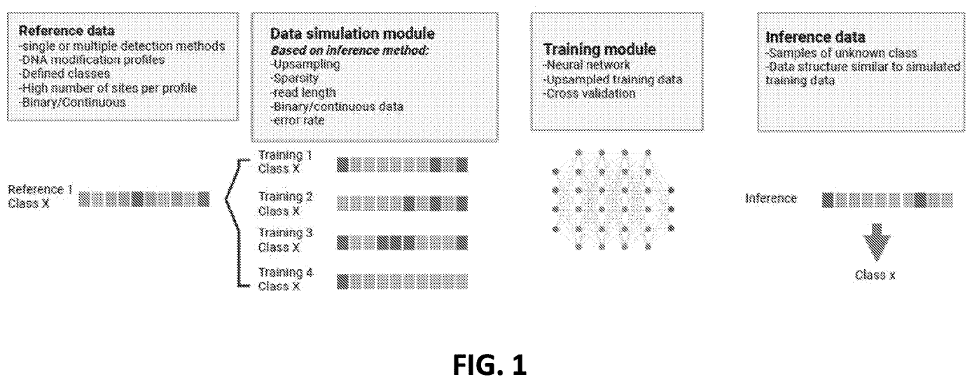

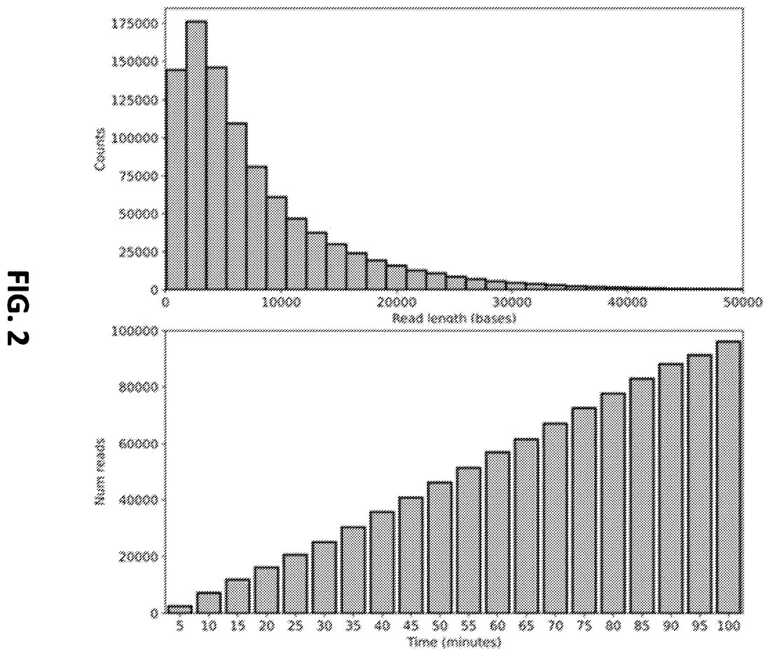

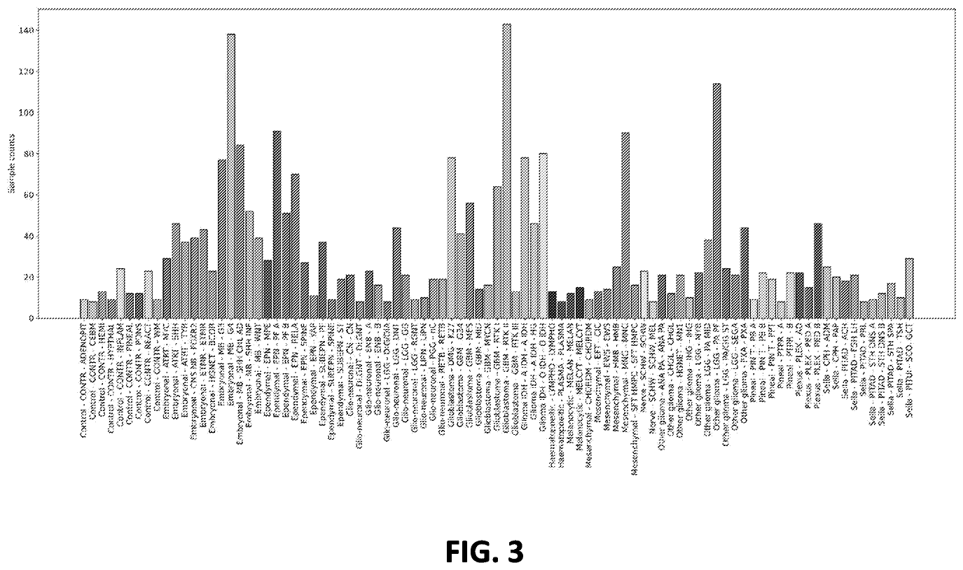

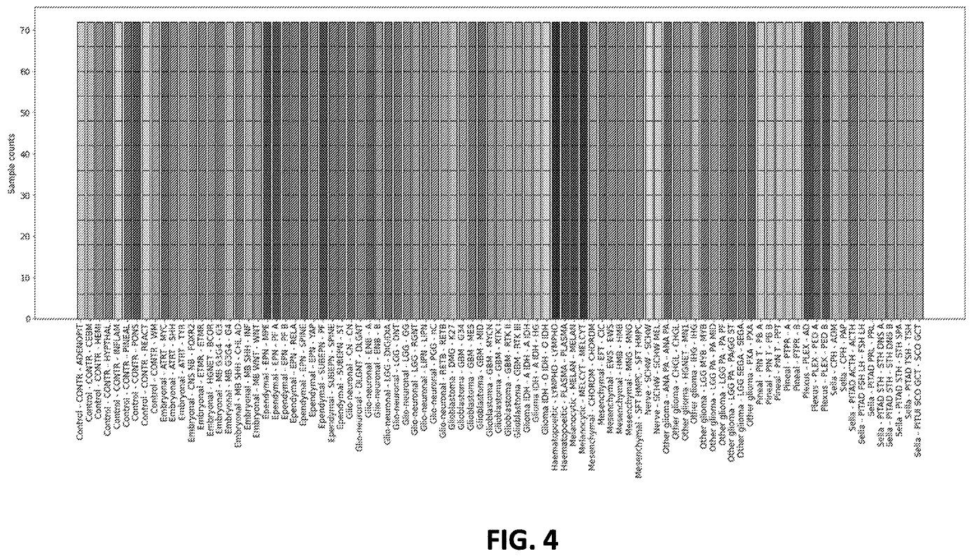

### DETAILED DESCRIPTION

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

The inventors found a method, more in particular, a computer-implemented method, comprising a DNA modification based deep learning (DL)-classifier (named “Sturgeon” by current inventors) comprising a one-size-fits-all sparse DNA modification classifier, preferably DNA methylation classifier, that can be applied to data while it flows from a nanopore sequencer in real time. The current computer-implemented method does not require computationally intensive (re)training of a classifier based on the patient specific sequencing results, and thereby takes away a major time-consuming step in the workflow. The method uniquely enables TATs of around 60 minutes (from sample to result), which makes it compatible with an intraoperative setting, preferably intraoperative diagnostic setting.

The classifier can build the algorithmic model in several days of computational time in which creating, training and/or configuring the model is performed. However, once created, it can be applied to new modification, preferably methylation, profiles in a matter of seconds. Innovatively, the method in accordance to the invention employs a data simulation strategy which enables it to learn from the vast amount of microarray-based data available, while retaining its accuracy on (much sparser) nanopore data.

This is important, as large well-annotated nanopore datasets are currently lacking and will take years to reach the comprehensiveness of the available microarray datasets. This approach also allows to train multiple models for different classification systems, and to apply them in parallel in a clinically relevant timeframe.

In an illustrative example, current method is similar to detecting, say, a cat in internet images whilst 95-98% of the pixels in the image are missing. To achieve this, current method employs a deep learning architecture that is extremely robust to missing data and performs a data subsampling/augmentation approach to vastly expand the training dataset.

In one embodiment, the current invention comprises:
(a) A DNA modification based deep learning (DL)-classifier that takes as input reference data that contains DNA modification profiles (the modification state for locations in the reference genome) linked to cell types/states for the sample of origin to be ultimately detected.
(b) The DNA modification based deep learning (DL)-classifier then generates training data, from said reference data, into methylation profiles similar to those that will be obtained during inference. The DNA modification based deep learning (DL)-classifier then trains a classification model on the training data and is subsequently able to classify inference data regardless of its dissimilarity to the original reference data.
(c) DNA modification based deep learning (DL)-classifier is applied on inference data.

In an embodiment, the reference data may comprise molecular profiles for human subject derived samples, for instance tumor samples, and/or samples from other tissues and/or diseases. The molecular profiles used for the DNA modification based deep learning (DL)-classifier preferably reflect the DNA modification (for example DNA methylation or hydroxymethylation) state of a multitude of DNA sites in a reference genome. Any types of modifications are contemplated and encompassed herein. Each patient-derived profile is associated to one or more class-labels identifying each profile as being derived from a specific class (for example cancer type, immune response type, developmental stage, disease type).

In one, non-limiting, example, the profiles may be derived from a tissue sample, and therefore reflect the modification, preferably methylation, state of multiple cell populations in the sample, also comprising contamination by healthy cell material. In the case of a tumor sample, the tumor fraction may vary, and may be unknown, but should be larger than zero (unless the profile is from the ‘health’ or a ‘control’ class). Profiles can be derived from any number of a priori defined specific sites, for example in the Illumina Infinium arrays captures 450 k CpG sites in the genome, whereas the EPIC arrays capture 850 k sites. Some of the sites/measurements may be missing at random (due to dropout or technical issues) or be incorrect (due to measurement noise). Alternatively, and/or additionally, profiles may be derived from targeted sequencing strategies, which aim to capture methylation at a limited set of predefined sites in the genome.

Alternatively, and/or additionally, profiles can be obtained through a genome-wide approach; meaning that methylation calls are obtained at random (typically many or even all) sites in the genome, for example by genome-wide bisulfite sequencing or Nanopore sequencing.

As described herein, reference data may comprise a combination of profiles, e.g., profiles obtained using different methods, i.e. microarray data in combination with sequencing data may be used, complete or incomplete data may be used, etc. For example, the reference data comprises microarray data and the sequencing data comprises nanopore sequencing data. In another non-limiting example the reference data may comprise nanopore sequencing data and the sequencing data also comprises nanopore sequencing data.

Moreover, profiles can be measured as binary values (methylated vs unmethylated) and/or as continuous values (x % of observations of a genomic site are methylated).

In an embodiment, the reference data can be converted into training data by simulating profiles as similar as possible as they are obtained in the target procedure. The DNA modification based deep learning (DL)-classifier (Sturgeon) in accordance to the method of the invention can take sparsity into account (i.e. many measurements (sites in the genome) are missing at random), where only a fraction of the sites covered in the reference data is covered in the target data. The amount and randomness of the sparsity is modelled according to the target procedure.

### Examples

Nanopore sequencing: read length will influence the amount of sparsity and the randomness because, the longer the reads, the more sites you can cover; but also the less random they are as they will be adjacent to each other. Sequencing time will influence the amount of sparsity since the more you sequence the more sites you can cover.
Shallow bisulfite sequencing: reads are usually short, so fewer sites covered (more sparse), but they will be more evenly distributed throughout the genome, therefore randomness is increased.

The DNA modification based deep learning (DL)-classifier in accordance to the method of the invention can take random data collection into account, where an a priori unknown collection of reference sites are covered in the inference data.

The DNA modification based deep learning (DL)-classifier in accordance to the method of the invention can take sequence read length into account, where adjacent sites in the genome are more likely to be simultaneously covered. For example in nanopore sequencing, or e.g., PacBio sequencing (PacBio SMRT sequencing) reads can be 5 kB in length on average, and thus all sites within 5 this 5 kB window are detected simultaneously. With Illumina sequencing, reads are 50-300 bp in length and fewer adjacent sites are covered per read.

The DNA modification based deep learning (DL)-classifier in accordance to the method of the invention can take the binary vs continuous data into account to match the intended inference data.

The DNA modification based deep learning (DL)-classifier in accordance to the method of the invention can take inaccuracy and/or measurement noise into account, where modification calls may be faulty in a percentage of observations. The DNA modification based deep learning (DL)-classifier in accordance to the method of the invention can take platform-specific error rates into account during data simulation. Error rate can come from different sources depending on the platform. If known, noise properties can be modelled and included in the simulation.

### Examples

Nanopore sequencing: detection of methylation from the noisy measured electric signal.
Bisulfite sequencing: enzymatic conversion rate.

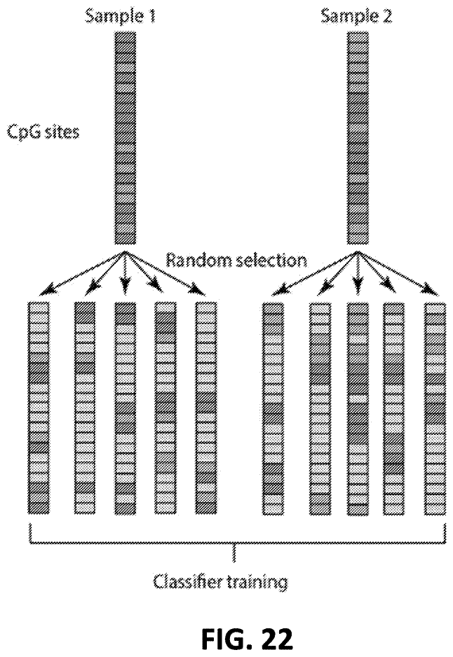

Preferably, due to the fact that a single training sample is a sparse subsampling from a reference data, many training samples can be created from a single reference sample. In addition, the amount of sparsity can be varied. As a result, the training data of the DNA modification based deep learning (DL)-classifier in accordance to the method of the invention can be majorly upsampled (by a factor of several thousand, even millions of times). This thus represents a powerful data augmentation procedure that greatly facilitates robustness when applied to new and unseen samples on inference. Since only a fraction of the methylation array data is used, the sampling can be performed multiple times to upsample the number of “patients” per diagnosis class. This enables to create a very large set (>10.000 profiles) of simulated runs, preferably nanopore runs, for training and validation (See FIG. 22). Moreover, this procedure allows to balance and/or control the number of training samples per class and sequencing depth.

Due to the fact that enormous amounts of training data can be made available (due to the upsampling) and that from the abundantly available reference data any desirable target data can be simulated, a very robust and accurate classification model can be trained. The DNA modification based deep learning (DL)-classifier preferably is trained on the simulated data and tested on simulated data as well as real inference data.

The DNA modification based deep learning (DL)-classifier is applied to classify newly generated target data containing DNA modification profiles derived from samples with an unknown class. Such data preferably comprises a modification profile structure similar to the training data.

In short, the current invention provides methods and means for identifying a DNA modification, preferably DNA methylation, in DNA sequencing data, for the intraoperative classification of a disease and/or disorder, preferably a tumor, more preferably a brain tumor, of a subject on which the operation is performed. One advantage of the current invention is that it can be performed intraoperatively, preferably within 2 hours. Potentially a classification and/or diagnosis can be reached approximately an hour after a biopsy is taken. The current invention further can provide an intra-operative (during the surgery) molecular classification of a disease and/or disorder, preferably of a tumor, thereby impacting the surgical strategy. In one preferred embodiment there is provided a machine learning framework for ultrafast cancer diagnoses.

The inventors, by means of the present invention, demonstrate that by using a DNA modification based deep learning (DL)-classifier a molecular classification for a disease and/or disorder can be obtained very fast, preferably even intraoperatively, namely during the operation of the subject of whom a sample, e.g., a tumor sample is obtained for said molecular classification.

The inventors have obtained preliminary results as disclosed in the examples and figures provided herein.

In a first aspect of the invention there is provided for a computer-implemented method for identifying a DNA modification, preferably DNA methylation, in DNA sequencing data, for the intraoperative classification of a disease and/or disorder, preferably a tumor, more preferably a brain tumor, of a subject on which the operation is performed, the method comprising the steps of:
a) obtaining DNA sequencing data of the subject, preferably wherein the DNA sequencing data is obtained by using a sequencing method capable of directly sensing a DNA modification, more preferably nanopore sequencing;
b) identifying the DNA modification status of the DNA sequencing data, thereby obtaining a DNA modification profile;
c) classifying the DNA modification profile using a DNA modification based deep learning (DL)-classifier thereby obtaining a classification score for a disease and/or disorder;
wherein the DNA modification based DL-classifier is trained with a training set, wherein the training is performed in a pre-operative training routine; and wherein the pre-operative training routine comprises training the DNA modification based DL-classifier on a training set generated from reference data comprising DNA modification data, preferably wherein the DNA modification data is any one or more selected from the group of microarray data, preferably hybridization microarray data, whole genome bisulfite sequencing data, TAPS-sequencing data, PacBio SMRT sequencing data or nanopore sequencing data.

It is contemplated that a sequencing method capable of directly sensing a DNA modification comprises pre-operative and/or intraoperative sequencing methods such as, microarray, (bisulfite) sequencing, TAPS-sequencing or nanopore sequencing. For example, in a pre-operative setting the sequencing comprises microarray, (bisulfite)
sequencing, PacBio SMRT sequencing data, TAPS-sequencing or nanopore sequencing, and in an intraoperative setting sequencing may comprise (bisulfite) sequencing, methods capturing sequence information during the replication process of the target DNA molecule, PacBio sequencing, NGS, TAPS-sequencing or nanopore sequencing. Preferably the sequencing method capable of directly sensing a DNA modification comprises nanopore sequencing (e.g., due to the speed), or other, fast (and therefore more shallow) sequencing approaches. A skilled person is aware of suitable alternative sequencing methods.

As provided herein the DNA modification based deep learning (DL)-classifier comprises a classifier based on a deep learning architecture, e.g., a neural network. The classifier is trained with a training set generated from reference data comprising DNA modification data and as described herein.

In another aspect of the invention there is provided for a computer-implemented method for identifying a DNA modification, preferably DNA methylation, of a sample of a subject, said sample comprising DNA for the intraoperative classification of a disease and/or disorder, preferably a tumor, more preferably a brain tumor, of a subject on which the operation is performed, the method comprising the steps of: (a) providing the sample comprising DNA; sequencing the DNA of the sample, preferably by using a sequencing method capable of directly sensing a DNA modification, more preferably nanopore sequencing, thereby obtaining DNA sequencing data; (b) identifying the DNA modification status of the DNA sequencing data, thereby obtaining a DNA modification profile; (c) classifying the DNA modification profile using a DNA modification based deep learning (DL)-classifier thereby obtaining a classification score for a disease and/or disorder; wherein the DNA modification based DL-classifier is trained with a training set, wherein the training is performed in a pre-operative training routine; and wherein the pre-operative training routine comprises training the DNA modification based DL-classifier on a training set from reference data comprising DNA modification data, preferably wherein the DNA modification data is any one or more selected from the group of microarray data, preferably hybridization microarray data, whole genome sequencing data, whole genome sequencing bisulfite sequencing, PacBio SMRT sequencing data, TAPS-sequencing data or nanopore sequencing data.

A sequencing method capable of directly sensing a DNA modification preferably comprises a method as disclosed herein, more preferably comprises nanopore sequencing. It is contemplated that the obtaining of DNA sequencing data can be done in one location (e.g. the location, such as the hospital, where the clinical procedure, preferably a surgical procedure, is performed on the subject whose disease and/or disorder, preferably a tumor, more preferably a brain tumor that benefits from intraoperative classification) and the sequenced data can be classified at the same and/or another location (e.g. another place in the hospital, remote of the hospital, by a local or remote server, web-based interface, SaaS or the like).

In an embodiment there is provided for the computer-implemented method in accordance with the present invention, wherein the reference data comprising DNA modification data is obtained by using a technique that differs from the technique used for obtaining DNA sequencing data of the subject and/or of the sample, preferably wherein said sample is obtained intraoperatively.

It is preferential that the reference data is obtained by using a different technique, preferably a different method for obtaining modification data, preferably a DNA modification profile, e.g., a DNA methylation profile, than the technique or method used for obtaining DNA sequencing data. Optionally, a combination of a different technique as described above may be used, and a technique that is similar and/or the same as the technique or method used for obtaining DNA sequencing data. In one preferred embodiment the reference data is obtained by using microarray, e.g., hybridization microarray, and the DNA sequencing data is obtained by using nanopore sequencing. If for example, in the future a larger number of data may be obtained through the nanopore and the target data is obtained from Nanopore as well. In such example, the method may apply despite both datasets being from the same technique. The reason why this method would still be suitable, is due to the fact the target sequencing would still be very shallow, hence, still shallow data would still be simulated from the reference data.

In an embodiment there is provided for the computer-implemented method in accordance with the present invention, wherein the pre-operative training routine comprises: (a) providing reference data comprising DNA modification data, preferably whole genome sequencing data, as input in a simulation module, preferably wherein the DNA modification data comprises a DNA modification profile associated with a cell type and/or cell state of interest; (b) using the simulation module to generate a training set from the reference data, wherein the training set comprises data having a DNA modification profile, preferably a nanopore profile; (c) training the DNA modification based DL-classifier on the training set.

It is contemplated that the DNA modification based DL-classifier is able to classify any DNA modification profile, preferably any DNA methylation profile for any cell type, cell state or disease and/or disorder. It is contemplated that the reference data should comprise DNA modification data of at least one cell type and/or cell state of interest to provide for suitable data for simulating a training set.

In an embodiment there is provided for the computer-implemented method in accordance with the present invention, wherein generating the training set from the reference data comprises any one or more of: (a) Binarization; (b) Non-uniform subsampling; and (c) Error simulation. preferably, wherein non-uniform subsampling comprises the random selection of CpG sites and the extension of the size of the sequencing reads and/or the sequencing time, and/or, preferably wherein error simulation comprises the generating of an error-rate of at least 10% in DNA modification-calling.

It is preferred that error simulation comprises the generating of an error-rate of, with increasing preference, at least 1%, at least 5%, at least 10% in DNA modification-calling. For example, the error-rate may be between 0.1% and 99.9%, preferably between 1% and 99%, or even 5% and 95%, or any value there in between, such as, at least 10%, 15%, 20%, 25%, 30%, 35%, 40%, 45%, 50%, 60%, 70%, 75%, 80%, 85%, 90%, 95%. Any suitable error rate may be used including any rate between 0 and 0.1%. For example, generating the training set from reference data may be used without error simulation, which may be achieved by setting the error rate to 0% or by not executing the error rate generation code.

It is preferred that an error simulation is achieved that more or less resembles nanopore sequencing data, i.e., the corresponding error-rate of nanopore sequencing data. It is contemplated that for various uses, methods and/or diseases and/or disorders different error-rates are preferred, e.g. error rates of at least 1%, 2%, 3%. To create the current classifier model, preferably one that can handle the sparsity challenge, a simulation module consisting of any one or more, preferably all three, components was developed to transform the reference data into the training set, preferably nanopore-like profiles:
Component 1—binarization: 450K and 850K arrays yield continuous data (beta values), representing methylated fraction of each site in the investigated population of cells. Nanopore data, on the other hand, yields a call for each CpG site in every sequenced read. In most cases this means methylation (Meissner et al.) data in nanopore experiments is binary: a site is either methylated or unmethylated. Reference data was converted to binary profiles using a cutoff of >0.6 for the Beta values. Accordingly, binarization refers to converting continuous data values (e.g., beta values) obtained for various sites to respective binary indications for the sites indicating, for each particular site, whether that site is methylated or unmethylated. For example, binarization may involve converting continuous data values for sites to respective binary values for the sites, with a particular binary value for a site indicating whether that site is methylated or unmethylated. The binary values may “0” and “1”, “−1” and “1”, “M” and “U”, or any other suitable pair of values, with one value of the pair indicating that the site is methylated and the other value of the pair indicated that the site is not methylated.
Component 2—non-uniform subsampling: Nanopore sequence reads are long, averaging ˜5 kB in the rapid sample prepping methods used in an intraoperative setting. As a result, the analyzed CpG sites are not individually sampled from the genome, but in series of adjacent CpG sites. As the methylation state between adjacent sites is correlated (Meissner et al.), this may negatively influence the efficiency. “Covered regions” were simulated by randomly selecting a location and extending the “coverage” based on the size of randomly selected reads from a nanopore sequencing experiment. Extending the coverage may include starting from a randomly selected location or locations and generating a sequence read by including, in the generated sequence read, the selected location(s) and its neighboring locations so that the total number of selected locations results in a synthetic read having a target length (e.g., always or on average). The target length may be set in accordance to an expected nanopore read length (e.g., ˜5 kB) appropriate for the nanopore sequencing technology whose sequence reads are being emulated through simulation.
Component 3—error simulation: Nanopore sequencing is error prone, and may result in some mis-calls, conservatively estimated to be 10%. Therefore, to generate training data, a corresponding noise as part of the data simulation module was included.

As described herein, reference data may comprise a combination of profiles, e.g., profiles obtained using different methods, i.e. microarray data in combination with sequencing data may be used, complete or incomplete data may be used, etc. Moreover, profiles can be measured as binary values (methylated vs unmethylated) and/or as continuous values (a fraction of observations of a genomic site are methylated).

In an embodiment there is provided for the computer-implemented method in accordance with the present invention, wherein the pre-operative training routine further comprises after c) the step d), wherein step d) comprises an intraoperative validation routine comprising validation of the DNA modification based DL-classifier on whole genome sequencing data, preferably nanopore sequencing data.

In an embodiment there is provided for the computer-implemented method in accordance with the present invention, wherein the pre-operative training routine comprises down sampling of the DNA modification data, preferably whole genome sequencing data and/or wherein the classifying of the DNA modification profile comprises up sampling of the whole genome sequencing data, preferably nanopore sequencing data.

Preferably, the DNA modification data may be downsampled, and/or preferably the whole genome sequencing data may be upsampled. Downsampling and/or upsampling may be done to even the unbalance in the data types as the DNA modification data may be over represented whereas the whole genome sequencing data may be underrepresented. Hence, with the upsampling (synthetic) generated data elements of the sample may be added to the dataset, whereas with downsampling (which may also be termed undersampling or subsampling), those data elements of the sample that are over-represented are reduced.

Accordingly, in some embodiments, DNA modification data obtained using a methylation profiling microarray (e.g., a microarray for profiling methylation at hundreds of thousands of CpG sites, such as between 400K and 500K sites or between 800K and 900K sites), may be upsampled such that a single set of DNA modification data can be used to generate any suitable number of simulated nanopore reads (e.g., at least 1K reads, at least 5K reads, at least 10K reads, at least 25K reads, at least 50K reads, at least 100K reads, at least 250K reads, between 5K and 50K reads, between 20K and 25K reads, between 10K and 40K reads, or any other suitable range within these ranges). In some embodiments, the number of simulated reads may be set based on the length of a desired sequencing run. For example, for a 20 minute sequencing run, 20-25K sequence reads may be simulated (e.g., approximately 1K reads per simulated sequencing minute).

In some embodiments, when DNA modification data obtained using a methylation profiling microarray is used to simulate nanopore reads, each simulated nanopore read may indicate methylation status for a smaller number of CpG sites than does the DNA modification data obtained using the microarray. In that sense, a simulated nanopore read may be considered to downsample the DNA modification data—it indicates methylation status for only a subset of the microarray CpG sites, which subset may be selected at random (though, as noted elsewhere, selected CpG sites may be correlated). When the number of CpG sites sampled by a simulated nanopore read is substantially smaller than the total number of CpG sites covered by the microarray (e.g., less than 15%, less than 10%, less than 5%, less than 4%, less than 3%, less than 2%, between 0.001 and 5%, between 0.001% and 4%), the sampling may be considered “sparse”.

Several methods are known for down and for upsampling, and the skilled person will appreciate which methods will be suitable and may be selected to pre-process the data for the DL-classifier according to the present disclosure.

In an embodiment there is provided for the computer-implemented method in accordance with the present invention, wherein the DNA modification classification provides a diagnosis of a tumor species, preferably of a brain tumor species.

Preferably the disease and/or disorder comprises a tumor, more preferably a CNS tumor, or brain tumor, e.g., a glioblastoma. It is preferred that the obtaining/taking of a biopsy of the tumor of the subject as provided herein is complex and, preferably, the subject undergoes surgery, for example craniotomy. It is contemplated herein that the current computer-implemented invention provides for a means to quickly provide a DNA classification of a tumor.

In an embodiment there is provided for the computer-implemented method in accordance with the present invention, wherein the DNA sequencing data is obtained by whole genome sequencing, preferably by nanopore sequencing.

In an embodiment there is provided for the computer-implemented method in accordance with the present invention, wherein the sample comprising DNA is a sample, preferably a tumor sample, and wherein the sample is derived from a subject intraoperatively; and/or wherein the subject is a human subject, preferably a human subject having a disease and/or disorder, preferably a tumor, more preferably a brain tumor, and preferably wherein the subject is under surgery.

It is contemplated that the current computer-implemented method is suitable for classifying a DNA modification (profile) of a sample, e.g. a sample comprising DNA, intraoperatively, meaning that the classification is performed during an (surgical) operation, preferably during the surgical operation of a subject, preferably of the subject undergoing said operation.

For example, when surgery is performed on a subject or a patient, for example for removing a brain tumor, a biopsy may be taken from the subjects, e.g. a biopsy from the brain. The biopsy may then be prepared as a sample for sequencing. The skilled person will appreciate how such sample preparation may be done. Then, the sample is sequenced, for example through nanopore sequencing. The sequenced data may subsequently be fed to the DNA modification based DL-classifier for classification of the sample. The classifier performs a comparison and provides its outcome, e.g. as a classification score for a disease and/or disorder, in the present example, of the classification of the brain tumor. The outcome of the classifier may assist in determining whether in accordance with the classification, more radical surgery would be needed, or a more restrictive and defensive resection. The classification according to the present disclosure may be performed to historical assessment and can be a used for pathologists in performing histological assessments. Moreover, in an example, the step of sequencing may continue after surgery, for obtaining additional data, e.g. for copy number variation.

It is expressed that the computer-implemented method according to the present disclosure is suitable for intraoperative (molecular) classification, but is not limited to such intraoperative classification as the skilled person will appreciate that other applications in which only sequenced samples are available which are shallow and sparse and/or wherein time is on the essence and conventional DL-classifiers would require time-consuming sample or patient specific training of the model, which time is not available or fast classification is required.

It is expressed that a single, some or all steps of the method may be performed on-site, e.g. in or near the surgery, for example in an inhouse data-centre of a hospital, but may also be performed remotely, e.g. through a cloud-based service, Software as a Service solution, or terminal-client based solution. The skilled person will appreciate which centralized or decentralized implementations may be suitable.

It is contemplated that the subject can be any mammal, preferably a human.

In an embodiment there is provided for the computer-implemented method in accordance with the present invention, wherein the DNA modification data comprises a DNA modification selected from the group consisting of: methylation or oxidation. Preferably the DNA modification is DNA methylation.

It is preferred that methylation comprises any one or more of CpG methylation, GpC methylation, 4mC methylation, 6 mA methylation, 5mC methylation, 5-hydroxymethylation, preferably 5mC methylation. Preferably methylation is of (one or more nucleotides of) the DNA.

A method for identifying a disease and/or disorder, preferably a tumor, more preferably a brain tumor, comprising performing the computer-implemented invention in accordance to the present invention.

It is contemplated that any disease and/or disorder can be classified, identified and/or diagnosed by using the computer-implemented method in accordance to the invention. It is preferred that the disease and/or disorder requires obtaining a biopsy and/or a sample from a subject suffering from said disease and/or disorder and/or that a DNA modification profile is advantageous in the identifying of the disease and/or disorder. The disease and/or disorder preferably is a tumor. Any tumor may be suitable for identification by the method in accordance to the invention, such as a melanoma, meningioma, astrocytoma and the like. The skilled person is aware of other types of tumors as encompassed herein. Preferably the obtaining of a DNA modification profile is advantageous in the identifying and/or operating on the tumor. It is most preferred that the tumor is a brain tumor. Any brain tumor, such as the brain tumors listed in Table 2, is encompassed herein.

In another aspect of the invention there is provided for a method of the pre-operative configuring of a DNA modification based DL-classifier, preferably a DNA modification based DL-classifier as used in any one of the previous claims, to receive a sample comprising DNA and/or to receive DNA sequencing data and to generate a classification score for a disease and/or disorder, preferably for a tumor species, the method comprising: training a DNA modification based DL-classifier, which runs on a processor coupled to memory, comprising a pre-operative training routine using as input reference data, wherein the reference data comprises DNA modification data, and wherein the DNA modification based DL-classifier after training is configured to receive a DNA modification profile, and wherein the DNA modification based DL-classifier generates a classification score output that indicates whether the DNA modification of the DNA modification profile is indicative for a disease and/or disorder.

In an embodiment, there is provided for the method of the pre-operative configuring of a DNA modification based DL-classifier in accordance with the present invention, wherein the DNA modification data comprises any one or more selected from the group of microarray data, preferably hybridization microarray data, whole genome bisulfite sequencing data, TAPS-sequencing data or nanopore sequencing data.

In an embodiment there is provided for the method of the pre-operative configuring of a DNA modification based DL-classifier in accordance with the present invention, wherein the DNA sequencing data is obtained by using a sequencing method capable of directly sensing a DNA modification, more preferably nanopore sequencing

In an embodiment there is provided for the method of the pre-operative configuring of a DNA modification based DL-classifier in accordance with the present invention, wherein the reference data comprising DNA modification data is obtained by using a technique that differs from the technique used for obtaining DNA sequencing data of the subject and/or of the sample, preferably wherein said sample is obtained intraoperatively.

In another embodiment there is provided for computer-implemented method in accordance to the present invention or for a method in accordance to the present invention, wherein the disease and/or disorder is a cancer, preferably a tumor, preferably a brain tumor.

In an aspect of the invention there is provided for a method of treatment comprising the step of performing the computer-implemented method as disclosed herein. It is preferred that the method of treatment as provided herein comprises the steps of (a) performing the computer-implemented method as disclosed herein; and (b) treating a tumor, preferably a brain tumor.

In an alternative aspect there is provided for a method of surgery comprising the step of performing the computer-implemented method as disclosed herein. It is preferred that the method of surgery as provided herein comprises the steps of (a) performing the computer-implemented method as disclosed herein; and (b) removing a tumor, preferably a brain tumor by surgery, preferably by surgical resection.

All references cited herein are incorporated by reference in their entirety.

The preceding description is presented to enable the making and use of the technology disclosed. Various modifications to the disclosed implementations will be apparent, and the general principles defined herein may be applied to other implementations and applications without departing from the scope of the appended claims. Thus, the technology disclosed is not intended to be limited to the implementations shown.

The inventors have developed new machine learning techniques for identifying the type of a patient's tumor from DNA methylation data obtained by sequencing a sample of the patient's tumor. The machine learning techniques developed by the inventors are able not only to accurately identify the tumor from DNA methylation status at a small number of candidate DNA methylation sites (e.g., using less than 5% of CpG sites of approximately 450K CpG or 850K CpG sites present on an Illumina Infinium methylation array), but also to do so much more quickly than conventional techniques (e.g., with a turn-around time of 90 minutes or less, or even 60 minutes or less). The ability to quickly and accurately determine tumor type from DNA methylation status at a small number of candidate DNA methylation sites makes the approach developed by the inventors applicable in intra-surgical applications (e.g., surgery for removing central nervous system tumors), where time is of the essence.

Conventional microarrays can be used to determine presence or absence of DNA methylations at hundreds of thousands of potential sites, which would allow the construction of a complete and robust DNA methylation profile for a patient. However, using such arrays takes several days and cannot be used in a surgical setting. On the other hand, nanopore sequencing can be used in a surgical setting because it may be used to rapidly sequence a tumor sample and perform base and methylation calling. However, given the limited amount of time available during surgery, nanopore sequencing can generate DNA methylation status for only a small fraction of the hundreds of thousands of potential DNA methylation sites and it is a priori unknown which DNA methylation sites will be covered.

As a result, one conventional approach to using nanopore sequencing data to identify tumor type is to: (1) obtain some DNA sequencing data for the patient during the surgery; (2) training a random forest model to predict the patient's tumor type for the CpG sites covered by the DNA sequencing data; and (3) using the trained random forest model to predict the tumor type from the DNA sequencing data. A major issue with this approach is that the training step is a computationally-intensive step that must take place during the surgical procedure because random forest models could not and cannot be trained to process data from an unknown subset of CpG sites (the sheer number of combinations of sites for which this would have to be performed is a combinatorial explosion). And, indeed, until the technology developed by the inventors, there was no technique available for processing DNA methylation data for an a priori unknown set of CpG sites.

By contrast, the technology developed by the inventors allows for offline (e.g., prior to surgery) training of a machine learning model that, once trained, can be used to process sparse DNA methylation data (e.g., data indication DNA methylation status for only a small fraction of all potential CpG sites of interest) obtained from a patient during a surgery via nanopore sequencings. And it is not necessary to know in advance the potential CpG sites that will be covered by nanopore sequencing when applied to the tumor sample. The developed techniques allow for overall turn-around-time of 60 minutes (or less) from sample to result.

As a result, the technology developed by the inventors improves on conventional ML technology for identifying tumor type from DNA methylation data obtained by nanopore sequencing. The developed technology can be used in a surgical setting, which was not possible using conventional techniques.

Accordingly, an aspect provides for a method for identifying a type of tumor (e.g., a type of a central nervous system (CNS) tumor) in a patient based on DNA methylation status of a subset of a plurality of candidate DNA methylation sites, the method comprising: (A) obtaining DNA sequencing data previously obtained in part by (e.g., nanopore sequencing) sequencing a biological sample obtained from the patient (e.g., from the patient's tumor); (B) identifying, using the DNA sequencing data, the subset of the plurality of candidate DNA methylation sites as those DNA methylation sites for which the DNA sequencing data indicates DNA methylation status (e.g., by assigning methylation calls in the DNA sequencing data to microarray CpG sites); (C) generating a sparse DNA methylation profile for the patient using the DNA sequencing data, the sparse DNA methylation profile indicating DNA methylation status of sites in the identified subset of the plurality of candidate DNA methylation sites; and (D) identifying the type of tumor in the patient by processing the sparse DNA methylation profile using a trained neural network model to obtain output indicative of the type of the tumor in the patient. In a further aspect, there is provided a system at least one computer hardware processor; and at least one non-transitory computer readable storage medium storing processor-executable instructions that, when executed by the at least one computer hardware processor, cause the at least one computer hardware processor to perform said method. In a yet further aspect there is provided at least one non-transitory computer readable storage medium storing processor-executable instructions that, when executed by at least one computer hardware processor, cause the at least one computer hardware processor to perform said method.

Some embodiments provide for a system for identifying a type of tumor in a patient based on DNA methylation status of a subset of a plurality of candidate DNA methylation sites, the system comprising: a nanopore sequencing apparatus; and at least one computing device configured to perform: (A) obtaining DNA sequencing data at least in part by sequencing a biological sample obtained from the patient using the nanopore sequencing apparatus; (B) identifying, using the DNA sequencing data, the subset of the plurality of candidate DNA methylation sites as those DNA methylation sites for which the DNA sequencing data indicates DNA methylation status; (C) generating a sparse DNA methylation profile for the patient using the DNA sequencing data, the sparse DNA methylation profile indicating DNA methylation status of sites in the identified subset of the plurality of candidate DNA methylation sites; and (D) identifying the type of tumor in the patient by processing the sparse DNA methylation profile using a trained neural network model to obtain output indicative of the type of the tumor in the patient.

In some embodiments, sequencing the biological sample consists of sequencing the biological sample for an amount of time between 5 and 60 minutes (e.g., between 10 and 45 minutes, between 15 and 30 minutes, or any other suitable range within these ranges) to obtain the DNA sequencing data.

In some embodiments, the acts (A), (B), (C), and (D) may be performed while the patient is undergoing surgery. For example, a biological sample (e.g., of a patient's tumor) may be obtained and sequenced to obtain DNA sequencing data, which may be used to generate a sparse DNA methylation profile for the patient, all during the surgery. Moreover, during the surgery, the type of tumor for the patient may be identified by processing the sparse DNA methylation profile using a trained neural network. In turn, the identified type of tumor may be used to influence the manner in which the surgery proceeds. For example, the surgery may be stopped based on the identified tumor type (e.g., in favor of an alternative treatment such as a radiotherapy, chemotherapy, immunotherapy, etc.). As another example, the manner in which the surgery is conducted may be changed based on the identified tumor type (e.g., the surgery may be conducted more aggressively or less aggressively with respect to resecting the tumor).

In some embodiments, nanopore sequencing may be performed continuously such that more DNA sequencing data for the patient is generated over time. The sparse DNA methylation profile for a patient may be updated (e.g., according to a schedule), as additional DNA sequencing data becomes available, and the updated sparse DNA methylation profile may be reprocessed by the trained neural network model to see if the indication of the patient's tumor type is the same (or different) and/or whether the confidence in the indication is greater or lower than before.

Accordingly, in some embodiments, the output indicative of the type of tumor in the patient includes a confidence associated with the type of tumor indicated, and the method further comprises, while the patient is undergoing the surgery and when the confidence is below a predetermined threshold confidence, continuing to sequence the biological sample using the nanopore sequencing to generate additional DNA sequencing data; augmenting the sparse DNA methylation profile using information in the additional DNA sequencing data to obtain an augmented sparse DNA methylation profile; and identifying the type of tumor in the patient by processing the augmented sparse DNA methylation profile using the trained neural network model to obtain a second output indicative of the type of the tumor in the patient.

In some embodiments, the DNA sequencing data indicates DNA methylation status only for sites in the identified subset of plurality of candidate DNA methylation sites, and the identified subset consists of between 0.001% and 4.0% of sites in the plurality of candidate DNA methylation sites. In some embodiments, the plurality of candidate DNA methylation sites consists of between 400,000 and 500,000 sites or between 800,000 and 900,000 sites.

In some embodiments, the DNA sequencing data was obtained by nanopore sequencing; and the plurality of candidate DNA methylation sites consists of a number of sites substantially equal to a number of CpG probes in a methylation profiling microarray.

In some embodiments, generating the sparse DNA methylation profile comprises: generating a data structure representing the methylation profile, the data structure configured to store values for a plurality of entries corresponding to the plurality of candidate DNA methylation sites; and setting, based on the DNA sequencing data, values for a subset of the plurality of entries that correspond to the identified subset of the plurality of candidate DNA methylation sites, wherein a first value for a first entry in the subset of the plurality of entries indicates presence or absence of DNA methylation at a candidate DNA methylation site in the identified subset to which the first entry corresponds.

In some embodiments, values of entries, which are in the plurality of entries but not in the subset of the plurality of entries, indicate that DNA methylation status was not indicated by the DNA sequencing data for candidate DNA methylation sites to which the entries correspond.

In some embodiments, processing the sparse DNA methylation profile using the trained neural network model comprises processing values stored in the data structure using the trained neural network model.

In some embodiments, the subset of the plurality of entries in the data structure consists of between 0.001 and 4% of all entries in the data structure.

In some embodiments, the trained neural network comprises a plurality of fully connected layers with non-linear activations therebetween and a classification head.

In some embodiments, the trained neural network comprises: a first fully connected layer having a first input size corresponding to a total number of sites in the plurality of candidate DNA methylation sites and a first output size smaller than the input size; a second fully connected layer having a second input size equal to the first output size and a second output size smaller than the second input size; a third fully connected layer having a third input size equal to the second output size and a third output size smaller than the third input size and corresponding to a number of candidate types of the tumor; and a first non-linear activation between the first and second fully connected layers and a second non-linear activation between the second and third fully connected layers.

In some embodiments, the trained neural network comprises at least 10 million parameters, and processing the sparse DNA methylation profile using the trained neural network comprises determining the output indicative of the type of the tumor in the patient by using values in the sparse DNA methylation profile and values of the at least 10 million parameters.

In some embodiments, the output indicative of the type of tumor in the patient comprises a plurality of likelihoods corresponding to a respective plurality of tumor types, wherein each particular likelihood of the plurality of likelihoods indicates a likelihood that the patient has a respective particular type of tumor in the plurality of tumor types.

In some embodiments, sequencing the biological sample using nanopore sequencing comprises sequencing the biological sample using nanopore sequence with adaptive sampling. In some embodiments, sequencing the biological sample using nanopore sequencing comprises, while a DNA strand is being sequenced using a nanopore part of a nanopore sequencing apparatus, obtaining a partial sequence of the DNA strand using measurements generated by the nanopore; determining whether the partial sequence maps to at least one of the plurality of candidate DNA methylation sites; when it is determined that the partial sequence does not map to at least one of the plurality of candidate DNA methylation sites, ejecting the DNA strand from the nanopore; and when it is determined that the partial sequence maps to the at least one of the plurality of candidate DNA methylation sites, continuing to sequence the DNA strand.

In some embodiments, the techniques further comprise: generating training data using microarray methylation data; and training a neural network model using the training data to obtain the trained neural network model. In some embodiments, generating the training data using microarray methylation data comprises generating, from microarray methylation data, sparse DNA methylation profiles representative of types of sparse DNA methylation profiles that would be generated using nanopore sequencing with a threshold amount of time.

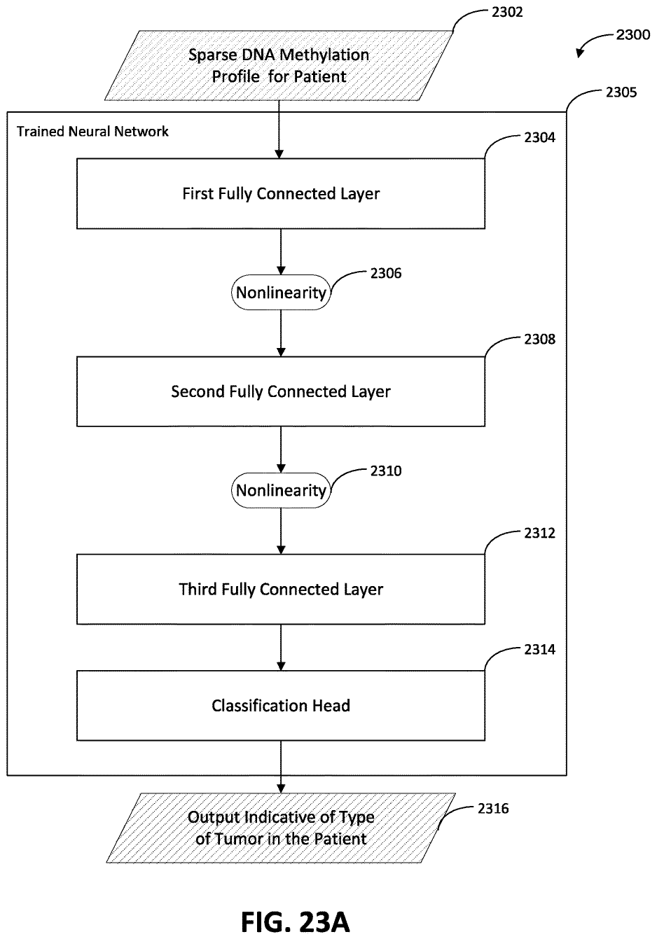

FIG. 23A is a block diagram 2300 illustrating an example architecture of a trained neural network 2305 for identifying a type of tumor in a patient by using the trained neural network to process a sparse DNA methylation profile 2302 for the patient to obtain an output 2316 indicative of the type of tumor in the patient, in accordance with some embodiments of the technology described herein.

As shown in FIG. 23A, trained neural network 2305 comprises a plurality of layers including first fully connected layer 2304, second fully connected layer 2308, and third fully connected layer 2312. In addition, trained neural network 2305 includes first nonlinearity 2306 between first fully connected layer 2304 and second fully connected layer 2308 and second nonlinearity 2310 between the second fully connected layer 2310 and third fully connected layer 2312. In the illustrative embodiment of FIG. 23A, the third fully connected layer 2312 is not followed by a non-linearity and so may be considered a “linear layer”. The third fully connected layer 2312 is followed by classification head 2314, which may be implemented using a softmax classifier, for example as shown in the embodiment illustrated in FIG. 23B.

As shown in FIG. 23A, sparse DNA methylation profile 2302 is provided as input to first fully connected layer 2304. Accordingly, in the illustrated embodiment, the first fully connected layer has an input size that is equal to the total number of entries in the sparse DNA methylation profile 2302. The total number of entries in the sparse DNA methylation profile 2302 may be the same as the total number of candidate methylation sites being monitored, which may be hundreds of thousands of sites (e.g., the number of sites may be within one or more of the following ranges 100K-1M sites, 100K-200K sites, 100K-500K sites, 400K-500K sites, 500K-1M sites, 700-900K sites, 800K-900K sites). As one specific example, shown in FIG. 23B, the total number of entries in the sparse DNA methylation profile may be 428643. Among the total number of entries, only a small percentage of entries (e.g., less than 5% of entries) may indicate the presence of a DNA methylation.

In some embodiments, including the embodiment shown in FIG. 23A, the output size of the first fully connected layer 2304 is smaller than its input size. For example, the output size of first fully connected layer may be at least order of magnitude (e.g., at least ten or a hundred times smaller) that its input size. As one specific example, shown in FIG. 23B, the output size of the first fully connected layer is 256, which is more than 100 times smaller than the input size of 428,643.

As shown in FIG. 23A, a first non-linearity 2306 is applied to the output of the first fully connected layer 2304. The first non-linearity 2306 may be a sigmoid, a hyperbolic tangent, a rectified linear unit (ReLU), a leaky ReLU, a softmax, a swish function, or any other suitable type of non-linearity. After the first non-linearity 2306 is applied to the output of the first fully connected layer 2304 the result is provided as input to the second fully connected layer 2308. Accordingly, the second fully connected layer 2308 may have an input size equal to the output size of the first fully connected layer 2304. For example, as shown in FIG. 23B, the input size of fully connected layer 2356 is equal to the output size of fully connected layer 2354.

As shown in FIG. 23A, a second non-linearity 2310 is applied to the output of the second fully connected layer 2308. The second non-linearity 2310 may be a sigmoid, a hyperbolic tangent, a rectified linear unit (ReLU), a leaky ReLU, a softmax, a swish function, or any other suitable type of non-linearity. The second non-linearity 2310 may be the same type of non-linearity as first non-linearity 2306 (e.g., both may be a sigmoid) or may be a different type of non-linearity than first non-linearity 2310 (e.g., one may be a sigmoid and the other a ReLU).

After the second non-linearity 2310 is applied to the output of the second fully connected layer 2308 the result is provided as input to the third fully connected layer 2312. Accordingly, the third fully connected layer 2312 may have an input size equal to the output size of the second fully connected layer 2308. For example, as shown in FIG. 23B, the input size of fully connected layer 2358 is equal to the output size of fully connected layer 2356.

As shown in FIG. 23A, output of the third fully connected layer 2312 is provided to classification head 2314, which in turn generates output 2316 indicative of the type of tumor in the patient. The classification head 2314 may be implemented in any suitable way. For example, in some embodiments, the classification head 2314 may be implemented using a softmax scaling, for example, by applying the softmax scaling to output of the third fully connected layer. As another example, the classification head 2314 may be implemented using a softmax scaling applied to temperature-scaled output of the third fully connected layer (e.g., as shown in the example of FIG. 23B).

In some embodiments, the output 2316 comprises a plurality of likelihoods (e.g., probabilities) corresponding to a respective plurality of tumor types, where each particular likelihood of the plurality of likelihoods indicates a likelihood that the patient has a respective particular type of tumor in the plurality of tumor types. For example, as shown in the example of FIG. 23B, the output may include multiple (e.g., 91 or 30) probabilities corresponding to multiple respective tumor types (e.g., 91 types of brain tumors or 30 types of brainstem tumors), where each particular indicates a probability (as estimated by the neural network) that the patient has a respective particular type of tumor (e.g., one of the 91 or 30 tumor types) in the multiple tumor types.

The trained neural network may be trained to determine likelihoods that a patient's tumor belongs to any desired set of tumor types. For example, in some embodiments, the set of tumor types may be some or all of the tumor types used in any version of the brain classifier described by Capper D, Jones D T W, Sill M, Hovestadt V et al., Nature. 2018 Mar. 22; 555(7697):469-474., which is incorporated by reference in its entirety herein. For example, the set of tumor types may be some or all of the tumor types used in version 12.3 of the brain classifier made available at https://www.molecularneuropathology.org/mnp/classifiers/14, which is incorporated by reference in its entirety.

It should be appreciated that the neural network architecture shown in FIG. 23A is illustrative and that there may be variations. For example, one or more additional layers may be added to the neural network architecture (e.g., one or more fully connected layers, a batch normalization layer, a convolutional layer, and/or any other type of neural network layer). As another example, a transformer-based neural network architecture may be considered.

The trained neural network 2305 may be trained as a solution to a supervised multi-class classification problem. The parameters of the neural network 2305 may be learned from training data using any suitable neural network learning technique, such as stochastic gradient descent, for example, as implemented using optimization software. For example, the training software may implement the Adam optimizer described by Kingma, D. and Jimmy, B. (“Adam: A method for stochastic optimization.” arXiv preprint arXiv:1412.6980 (2014)). As another example, the training software may implement the AdamW optimizer described by Loshchilov, I. AdamW-and-SGDW: “Decoupled Weight Decay Regularization” (ICLR 2019).

The training data may comprise input-output pairs where, the input is a sparse DNA methylation profile for a patient and the output is a tumor type previously determined for the patient. As described herein, in some embodiments, one or more the sparse DNA methylation profiles in the training data may be simulated based on microarray data. Additionally or alternatively, one or more of the sparse DNA methylation profiles may have been obtained using nanopore sequencing (e.g., during surgery or otherwise).

FIG. 23B is a block diagram illustrating a specific example of a trained neural network 2355 having the neural network architecture shown in FIG. 23A, in accordance with some embodiments of the technology described herein.

As shown in in FIG. 23B, the trained neural network 2355 contains three fully connected layers 2354, 2356, and 2358. Each of the fully connected layers 2354 and 2356 are followed by a sigmoid activation. The input size of the fully connected layer 2354 is 428,643 corresponding to the number of probes on the microarrays used to generate sparse DNA methylation profiles part of the training data for training the neural network 2355. The output size of the fully connected layer 2354 is 256, which is the same as the input size of the fully connected layer 2356. The output size of the fully connected layer 2356 is 128, which is the same as the input size of the fully connected layer 2358. The fully connected layer 2358 is a linear layer in the sense, unlike layers 2354 and 2356, it is not followed by a sigmoid activation. The dimensionality of the fully connected layer 2358 is equal to the number of classes to be predicted. For example, the dimensionality of layer 2358 may be 91, in the case of predicting CNS tumor types generally, or 30, in the case of predicting brainstem-specific tumors.

In this example, the output of the fully connected layer 2358 is a vector of unscaled logits 2360, which is provided as input to classification head 2359. The unscaled logits 2360 are then divided by a learned scalar value (temperature parameter t in this example) to obtain scaled logits 2362. The temperature scaled logits 2362 are then softmax scaled to obtain per-class probabilities 2364, which indicate, for each of the output classes (e.g., 91 or 30 output classes) the probability that the patient has a tumor type corresponding to the output class.

Although in the example embodiment of FIG. 23B, the dimensionality of the output of fully connected layer 2356 (i.e., 128) is smaller than the dimensionality of its input (i.e., 256), in other embodiments, the output and input dimensions of the fully connected layer 2356 may be the same or the output dimension may be greater that (e.g., 512) than the input dimension (i.e., 256).

The trained neural network 2355 was trained using cross-entropy classification loss with uniform weights. Dropout rate between layers was set to 0.5. Aspects of training the trained neural network 2355 are described herein including in Example 6 below.

As shown in FIG. 23B, the trained neural network 2355 processes a sparse DNA methylation profile 2352 to obtain the output 2364 indicating, for each of multiple tumor types, a respective probability that the patient has that type of tumor. The sparse DNA methylation profile 2352 may be implemented using a data structure (e.g., one or more arrays, vectors, matrices, linked lists, etc.) configured to store values for a plurality of entries corresponding to a plurality of candidate DNA methylation sites (e.g., the DNA methylation sites with corresponding probes on a microarray used to generate training data to train the neural network 2355). In the sparse DNA methylation profile 2352, values for a subset of the entries may indicates presence or absence of DNA methylation at the candidate DNA methylation sites to which the subset of entries correspond. The size of that subset of entries may be between 0.001 and 4% of the total number of entries. The values rest of the entries (e.g., 96-99.999% of the entries) indicate that the DNA methylation status is not known (e.g., not indicated by the DNA sequencing data generated by nanopore sequencing) for the candidate DNA methylation sites to which the entries correspond.

In some embodiments, including in the embodiment illustrated in FIG. 23B, the sparse DNA methylation profile may be embodied in a vector of values in which the entry value of 1 indicates the presence of a DNA methylation at the candidate DNA methylation site to which the entry corresponds, the entry value of −1 indicates the absence of a DNA methylation at the candidate DNA methylation site to which the entry corresponds, and the entry value of 0 indicates the DNA sequencing data (e.g., generated by nanopore sequencing) provides no indication as to the DNA methylation status at the candidate DNA methylation site to which the entry corresponds.

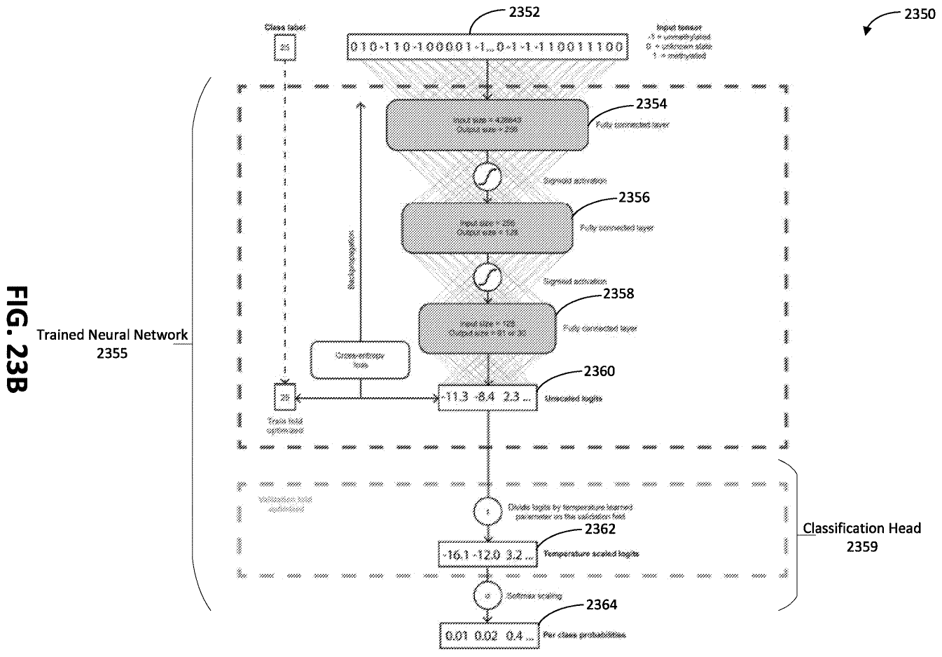

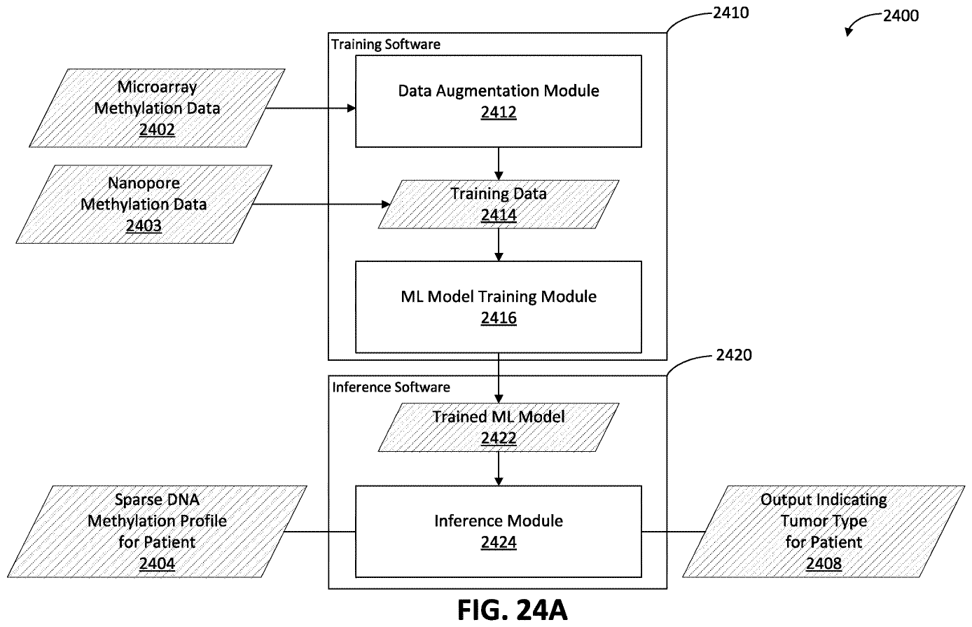

As described herein, aspects of the technology developed by the inventors may be implemented in software. This includes software for generating training data to train an ML model to predict a patient's tumor type, software for training such an ML model using the generated training data, and software for using the trained ML model to process previously-unseen sparse DNA methylation profiles. In some embodiments, the technology developed by the inventors may be implemented using software organized as shown in FIG. 24A, which is a block diagram of software 2400 for: (1) training a machine learning (ML) model to predict the type of a patient's tumor using a sparse DNA methylation profile generated for the patient; and (2) using the trained ML model to do so.

As shown in FIG. 24A, software 2400 comprises training software 2410 and inference software 2420. Training software 2410 may be used to generate training data for training an ML model (e.g., a neural network model, such as one of the models described with reference to FIGS. 23A and 23B) to predict the type of tumor that a patient has from a sparse DNA methylation profile for the patient. To this end, the training software 241 includes a data augmentation module 2412, which may be used to generate training data to augment any already-existing training data, and ML model training module 2416, which may be used to train a ML (e.g., a neural network model) using the training data.

In some embodiments, the data augmentation module 2412 may be configured to generate training data from microarray methylation data 2402. For example, data augmentation module 2412 may be configured to generate, from microarray methylation data 2402, sparse DNA methylation profiles, representative of the types of sparse DNA methylation profiles that would be generated using nanopore sequencing in a limited amount of time (e.g., in 10, 15, 20, 25, 30, 35, 40, 45, 50, 55, 60 minutes or less). As one example, N simulated reads may be randomly sampled from the read length distribution (D) and assigned a start mapping position in the genome. The values of N and D may be defined based on an average nanopore whole genome sequencing run using a MinION flowcell. Forward or reverse direction may be chosen at random (50% chance each). Reads may be clipped at the start/end of the chromosome. Given this set of reads, the covered CpG sites may be determined and their binarized methylation status may be obtained from the microarray sample. The microarray methylation data 2402 may be obtained by binarizing raw EPIC profiles with a cutoff of β≥0.6. To include measurement noise due to sample heterogeneity, and methylation calling error rate, 10% of the covered CpG sites may be randomly flipped.

In some embodiments, the sparse DNA methylation profiles generated by data augmentation module 2412 may be combined with any other available training data (e.g., nanopore methylation data 2403 which may include sparse DNA methylation profiles obtained directly by nanopore sequencing) to obtain training data 2414. Though it should be appreciated that other methylation data, such as nanopore methylation data 2403, may not be available and that only data generated by data augmentation module 2412 may be used to train the ML model.

More generally, it should be appreciated that the data augmentation module may be configured to generate a larger number (e.g., millions, tens of millions, hundreds of millions) of sparse DNA methylation profiles from other data to enable the training of the types of machine learning models (e.g., neural networks) described herein. As one example, the data augmentation module may be configured to generate a large number of sparse DNA methylation profiles from microarray methylation data. As another example, the data augmentation module may be configured to generate a larger number of shallow nanopore runs from a smaller number (e.g., thousands, tens of thousands, hundreds of thousands) deeper nanopore sequencing runs that have many times greater coverage of candidate methylation sites than do the shallow runs. In turn, the large number of shallow nanopore runs may be used to generate a large number of sparse DNA methylation profiles from which the machine learning models described herein may be trained.

In some embodiments, the ML model training module 2416 may use any suitable training algorithm(s) for training the ML model from the training data 2414 to obtain trained ML model 2422. For example, the ML model training module 2416 may implement training using a stochastic gradient descent technique, for example, as implemented using optimization software. For example, the training module 2416 may implement the Adam optimizer described by Kingma, D. and Jimmy, B. (“Adam: A method for stochastic optimization.” arXiv preprint arXiv:1412.6980 (2014)). As another example, the training software may implement the AdamW optimizer described by Loshchilov, I. AdamW-and-SGDW: “Decoupled Weight Decay Regularization” (ICLR 2019).

As shown in FIG. 24A, inference software 2420 may include inference module 2424, which may be configured to obtain a sparse DNA methylation profile 2404 for a patient (e.g., a previously unseen profile for a new patient, such as a patient undergoing surgery) and process the profile 2404 using trained ML model 2422 to obtain output 2408 indicating the tumor type for the patient.

In some embodiments, training software 2410 and inference software 2420 may be deployed in different computing environments. For example, training software 2410 may be deployed and executed in a cloud-computing or other computing environment to generate trained ML model 2422 and inference software 2420 may be deployed and executed in a different computing environment (e.g., a surgical environment, for example, surgical environment 2450 shown in FIG. 24B).

As described herein, the technology developed by the inventors may be applied in an intraoperative setting to facilitate typing of a patient's tumor. FIG. 24B is a block diagram illustrating a surgical environment 2450 in which the technology developed by the inventors may be used. Surgical environment 2450 may be an operating room or suite or any other environment suitable for performing surgery (e.g., surgery for removing brain or brain stem tumors) on patients.

As shown in FIG. 24B, surgical environment 2450 includes patient 2452 that may be undergoing surgery, one or more surgeons 2454 or other clinicians, and tumor type prediction system 2455. The tumor type prediction system includes nanopore sequencing apparatus 2461 and computing device(s) 2463, which may host software 2465 for generating, from DNA sequencing data, a sparse DNA methylation profile and processing it by using a trained ML model to output an indication 2470 of the type of the patient's tumor. The tumor type prediction system 2455 may generate an indication (with at least a desired level of confidence) of the tumor type within 60 minutes (e.g., within 60, 55, 50, 45, 40, 35 or 30 minutes). The surgeon(s) 2454 may adjust the manner in which they are performing surgery based on the indication 2470. For example, based on the indicated type of tumor, the surgery may be stopped (e.g., so that the cancer can be treated by one or more therapies instead of performing surgery, for example, radiation therapy, chemotherapy, or immunotherapy), be conducted less aggressively, or be conducted more aggressively, according to accepted clinical practices for tumors of the identified type.

While a patient is undergoing surgery, a sample 2456 of the tumor may be obtained from the patient and nanopore sequencing apparatus 2461 may be used to sequence the sample. The DNA sequencing data 2460 generated by nanopore sequencing apparatus 2461 may then be provided as input to software 2465. Within the software 2465, the DNA sequencing data 2460 may be processed by sparse DNA methylation profile sequencing module 2462 to generate a sparse DNA methylation profile 2464, as described herein. In turn, inference module 2466 processes the sparse DNA methylation profile using trained ML model 2468 (which, for example, may be any of the trained neural network models described herein including with reference to FIGS. 23A and 23B) to generate output 2470 indicating the patient's tumor type, as described herein.

Nanopore sequencing apparatus 2461 may be any suitable type of nanopore sequencer. For example, apparatus 2461 may be an Oxford Nanopore MinION device, an Oxford Nanopore MinION Mk1C device, an Oxford Nanopore GridION device, or an Oxford Nanopore PromethION device. As another example, any nanopore sequencing apparatus incorporating the technology described in U.S. Pat. No. 11,466,317, titled “Methods and Systems for Characterizing Analytes Using Nanopores”, granted Oct. 11, 2022, filed May 31, 2019, may be used. U.S. Pat. No. 11,466,317 is incorporated by reference herein in its entirety.

In some embodiments, tumor type prediction system 2455 may be used in “batch” mode. In this mode, nanopore sequencing apparatus 2461 may be used for a predetermined amount of time for sequencing the biological sample 2456. Subsequently, all the DNA sequencing data 2460 generated by the nanopore sequencing apparatus 2461 may be provided in one batch to the software 2465 for subsequent processing. The software 2465 may then process the entire batch of data according to the techniques described herein to provide the output 2470 indicating the patient's tumor type.

In some embodiments, tumor type prediction system may be used in “streaming” mode. In this mode, nanopore sequencing apparatus 2461 may provide DNA sequencing data 2460 to software 2465 on an ongoing basis as additional sequencing is performed and sequencing data becomes available. In this way, more and more DNA sequencing data is provided to the software 2465 over time as nanopore sequencing continues. As a result, over time, more information about DNA methylation status at individual candidate DNA methylation sites is provided to the software 2465. In turn, the software 2465 may update the sparse DNA methylation profile as further DNA sequencing data is available (e.g., by updating values of entries corresponding to newly sequenced sites to indicate whether DNA methylation is present or absent at those sites, for example, by setting the values to 1 and −1 to indicate presence or absence of DNA methylation). The updated sparse DNA methylation profile may be processed using the trained ML model 2468 to provide updated output 2470 indicating the patient's tumor type.

In some embodiments, the output 2470 indicates not only the patient's tumor type but also the confidence in that determination. For example, the output may indicate a particular type of tumor as the most probable type of tumor together with the associated probability. As more DNA sequencing data is provided to the software 2465, the confidence in the determination may change (e.g., increase or decrease). For example, initially, the output 2470 may indicate the patient's tumor type with a respective first probability. However, after additional data is received and processed, the updated output 2470 may indicate that the patient has the same type of tumor, but now with a respective second probability. The second probability may be higher than the first probability, indicating that the additional DNA sequencing data increased the system's confidence in the type of tumor identified. Alternatively, the second probability may be lower than the first probability, indicating that the additional DNA sequencing data decreased the system's confidence in the type of tumor identified. As yet another example, after additional data is received and processed, the updated output 2470 may indicate that the patient has a different type of tumor, which means that the initial determination was changed based on the additional DNA sequencing data.

In some embodiments, the tumor type prediction system 2455 may operate in streaming mode until it is able to identify the patient's tumor type with at least a threshold confidence. For example, the tumor type identification system may continue to process additional DNA sequencing data until the probability associated with the most probable type of tumor is greater than or equal to a threshold probability (which may be predetermined), for example, greater than 50%, 60%, 70%, 75%, 80%, 90%, 95%, 99%, 99.9% or any other suitable threshold probability. In some such embodiments, the updated DNA sequencing data may be provided to software 2465 according to a schedule and/or a predetermined rate (e.g., every thirty seconds, every minute, every 2, 3, 4, 5, 6, 7, 8, 9, or 10 minutes, etc.) until the system is able to identify the patient's tumor type with at least a threshold confidence or a total amount of type allotted for making such an identification has elapsed, whichever is earlier.

In the illustrative example of FIG. 24B, the nanopore sequencing apparatus 2461 and computing device(s) 2463 are shown as separate devices. The nanopore sequencing apparatus 2461 may be communicatively coupled (e.g., via a wired connection, via a wireless connection, or via a combination of wired and wireless connections) to the computing device(s) 2463. For example, the computing device(s) 2463 may be a laptop and the nanopore sequencing apparatus may be coupled via a cable to the laptop. In other embodiments, however, the nanopore sequencing apparatus may host software 2465 to generate sparse DNA methylation profiles from DNA sequencing data and process such profiles with a trained ML model to generate output indicating a patient's tumor type. In such other embodiments, the nanopore sequencing apparatus may include one or more processors that may execute the software 2465.

Although in the embodiment shown in FIG. 24B, a nanopore sequencing apparatus is used as part of the tumor type prediction system 2455, in other embodiments, one or more other types of sequencing devices may be employed.

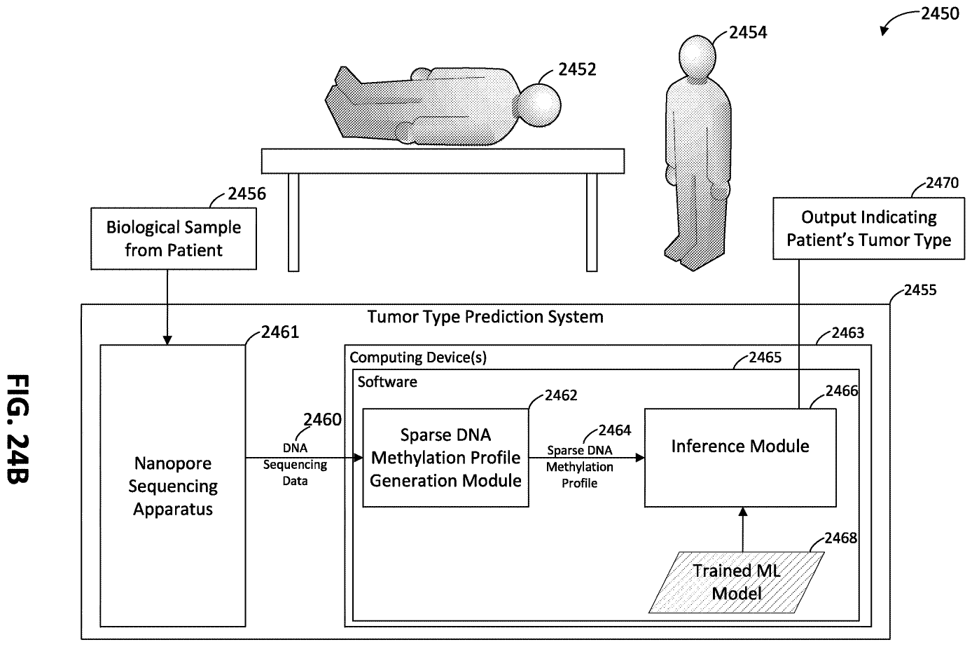

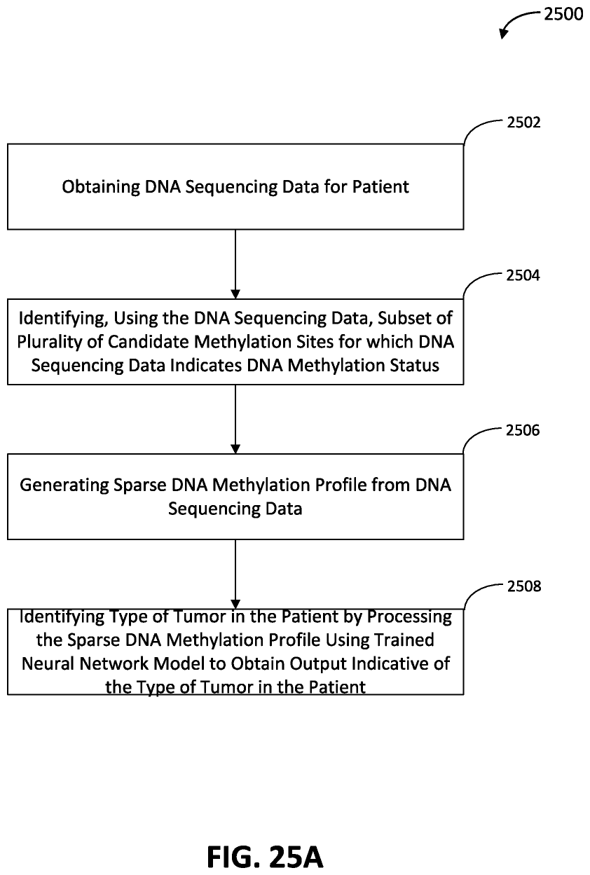

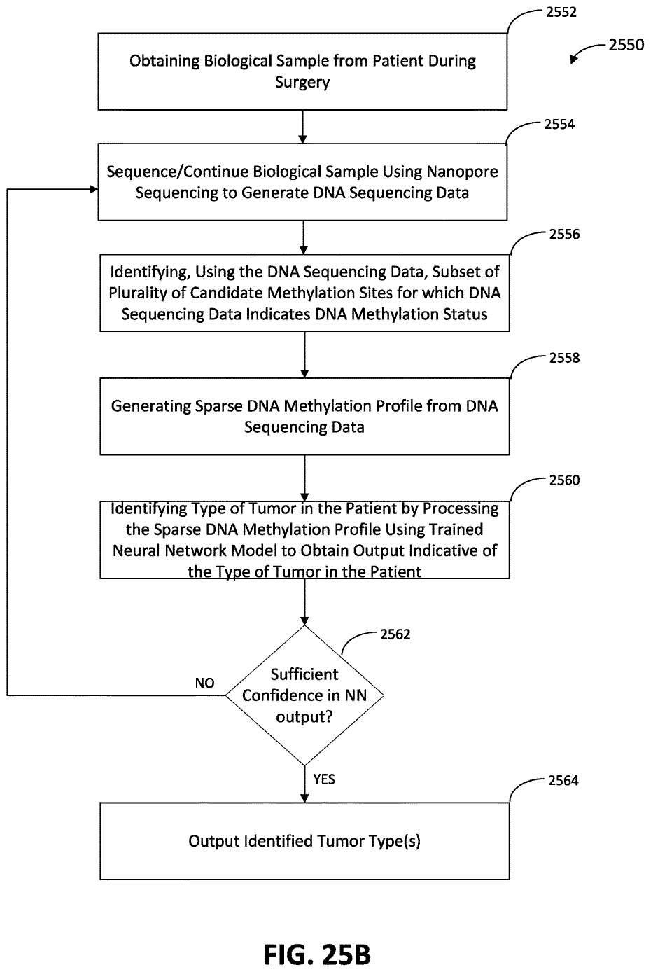

FIG. 25A is a flowchart of an illustrative process 2500 for predicting the type of a patient's tumor using a sparse DNA methylation profile generated for the patient from DNA sequencing data for the patient, in accordance with some embodiments of the technology described herein. Illustrative process 2500 may be performed by using any suitable computing device(s) and in any suitable environment. For example, in some embodiments, process 2500 may be performed using one or more computing devices part of tumor type prediction system 2455 (e.g., using computing device(s) 2163). As another example, in some embodiments, process 2500 may be performed by a nanopore sequencing apparatus (e.g., an apparatus that is configured to execute software for performing process 2500, for example, software 2465). In some embodiments, process 2500 may be performed using a stand-alone computing device (e.g., a laptop, a computer, a smart-phone). In some embodiments, process 2500 may be performed in a cloud-computing environment.

Process 2500 begins at act 2502, where DNA sequencing data for a patient is obtained. The DNA sequencing data may be DNA sequencing data generated using nanopore sequencing. The DNA sequencing data may include a plurality of DNA sequence reads obtained by performing base calling (using base calling software) on raw sequencing data generated by the nanopore sequencing apparatus. The base calling software may perform methylation calling. Accordingly, the DNA sequence reads may indicate sequences of DNA bases including methylated DNA bases (e.g., 5-methylcytosine).

For example, in some embodiments, the DNA sequencing data may be obtained by: (1) obtaining raw sequencing data from the nanopore sequencing apparatus (e.g., raw sequencing data in fast5 format); and (2) applying base-calling software (e.g., the Guppy or Megalodon software) to the raw sequencing data to obtain the DNA sequencing data. The DNA sequencing data may be in any suitable format. For example, the DNA sequencing data may be in FASTA or FASTQ formats. As another example, the DNA sequencing data in sequence alignment map (SAM) format, binary alignment map (BAM) format, or compressed reference-oriented alignment map (CRAM) format. Further aspects of generating DNA sequencing data are described herein including, an illustrative example described with reference to Example 6 in the sections titled “Methylation Calling” and “Live Analysis”.

In some embodiments, act 2502 may be performed entirely by a nanopore sequencing apparatus and software executing thereon. For example, the nanopore sequencing apparatus may generate raw sequencing data (e.g., in fast5 format) and apply base calling software (e.g., Guppy or Megalodon) to the raw sequencing data. In other embodiments, act 2502 may be performed in part using the nanopore sequencing apparatus and in part using one or more other computing devices. For example, the nanopore sequencing apparatus may generate the raw sequencing data and one or more other computing devices (e.g., a laptop computer connected to the nanopore sequencing apparatus) may apply the base calling software to the raw sequencing data provided to the other computing device(s) by the nanopore sequencing apparatus.

In some embodiments, the nanopore sequencing performed at act 2502 may be performed using adaptive sampling. In some such embodiments, sequencing the biological sample using nanopore sequencing comprises, while a DNA strand is being sequenced using a nanopore, obtaining a partial sequence of the DNA strand using measurements generated by the nanopore (e.g., a partial sequence of about 400 bases); determining whether the partial sequence maps to at least one of the plurality of candidate DNA methylation sites; when it is determined that the partial sequence does not map to at least one of the plurality of candidate DNA methylation sites, ejecting the DNA strand from the nanopore; and when it is determined that the partial sequence maps to the at least one of the plurality of candidate DNA methylation sites, continuing to sequence the DNA strand. Using such adaptive sampling can increase the number of CpG sites in a fixed amount of time (e.g., increasing the number of CpG sites sequenced per minute by as much as 15-30%).

Next process 2500 proceeds to act 2504 that involves identifying, using the DNA sequencing data (obtained at act 2502), a subset of a plurality of candidate methylation sites for which the DNA sequencing data indicates DNA methylation status. In some embodiments, the DNA sequencing data may be aligned to a reference and the resulting alignment may be used to determine the subset of a plurality of candidate methylation sites (in the reference) for which the sequencing data indicates DNA methylation status. In some embodiments, the plurality of candidate methylation sites may be the collection of CpG sites present in a methylation microarray (e.g., the approximately 450K CpG or 850K CpG sites present on an Illumina Infinium methylation array). The subset of the plurality of candidate methylation sites may be identified by assigning methylation calls in the DNA sequencing data to one of the sites present in the methylation microarray.

For example, in some embodiments, methylation calls may be assigned to one of the 450K CpG sites present on the Infinium methylation array using windows centered on the CpG site targeted by each probe. If multiple CpG sites are present within the window, majority voting may be used to convert the calls to a single call per site. When multiple reads cover the same probe site, majority voting may be also used to create one methylation call. When voting results in a tie, no methylation call may be made for that particular site.

In some embodiments, the total number of DNA methylation sites in the identified subset may be between 500 and 20,000 sites, between 1,000 and 10,000 sites, between 2,500 and 25,000 sites, between 500 and 50,000 sites, between 5,000 and 100,000 sites or any other range within these ranges.

Next, process 2500 proceeds to act 2506, where a sparse DNA methylation profile is generated using the DNA sequencing data. The generated sparse DNA methylation profile may indicate methylation status (e.g., the presence or absence of methylation) for the sites in the subset of sites identified at act 2504.

In some embodiments, generating the sparse DNA methylation profile comprises generating a data structure (e.g., a vector) representing the methylation profile, the data structure configured to store values for a plurality of entries corresponding to the plurality of candidate DNA methylation sites; and setting, based on the DNA sequencing data, values for a subset of the plurality of entries that correspond to the identified subset of the plurality of candidate DNA methylation sites. A value for a particular entry may indicate: (1) presence (e.g., when the value is equal to 1) of DNA methylation at a candidate DNA methylation site in the identified subset to which the particular entry corresponds; (2) absence (e.g., when the value is equal to −1) of DNA methylation at the candidate DNA methylation site in the identified subset to which the particular entry corresponds; or (3) that DNA methylation status was not determined from the DNA sequencing data for the candidate DNA methylation site in the identified subset to which the particular entry corresponds (e.g., either because the DNA sequencing data did not have any or a sufficient number of sequence reads covering the candidate DNA methylation site or because the sequence reads that were available did not sufficiently agree or were otherwise of low quality such that a methylation call for the site could not be reliably made using the DNA sequencing data).

As one specific example, in some embodiments, generating the sparse DNA methylation profile comprises generating a vector, where each vector value for a particular entry in the subset of the plurality of entries that corresponds to the identified subset of the plurality of candidate DNA methylation sites is either a 1, indicating presence of a DNA methylation, or −1, which indicates absence of a DNA methylation at the candidate DNA methylation site to which the particular entry corresponds. In addition, each vector value for a particular entry not in the subset of the plurality of entries that corresponds to the identified subset of the plurality of candidate DNA methylation sites, is set to 0 indicating that the DNA sequencing data provides no indication as to the DNA methylation status at the candidate DNA methylation site to which the particular entry corresponds.

Next, process 2500 proceeds to act 2508, which involves identifying the type of tumor in the patient by processing the sparse DNA methylation profile (generated at act 2506) using a trained neural network model (e.g., any of the neural network models described herein including with reference to FIGS. 23A and 23B) to obtain output indicative of the type of tumor in the patient. Processing the sparse DNA methylation profile involves processing the values stored in the sparse DNA methylation profile using the trained neural network model.

The trained neural network model may be trained prior to the start of process 2500. In some embodiments, the training neural network model may be trained using training data generated using microarray methylation data. Generating the training data may involve generating, from microarray methylation data, sparse DNA methylation profiles representative of types of sparse DNA methylation profiles that would be generated using nanopore sequencing with a threshold amount of time. Aspects of generating the training data are described herein including with reference to FIG. 24A and Example 6.

FIG. 25B is a flowchart of another illustrative process 2550 for predicting, while a patient is undergoing surgery, the type of the patient's tumor using a sparse DNA methylation profile generated from DNA sequencing data for the patient obtained by nanopore sequencing, in accordance with some embodiments of the technology described herein. The illustrative process may be performed in a surgical environment (e.g., surgical environment 2450 described with reference to FIG. 24B). One or more acts of process 2550 may be performed using tumor type prediction system 2455.

Process 2550 begins at act 2552 where a biological sample of a patient's tumor is obtained from the patient during a surgery. The sample is prepared for sequencing and a nanopore sequencing apparatus starts sequencing the sample at act 2554 to generate DNA sequencing data. The DNA sequencing data may be generated in any suitable way, including as described herein with reference to act 2502 of process 2500. In some embodiments, including in the illustrated embodiment, the DNA sequencing data may be generated using nanopore sequencing, though other DNA sequencing technology may be used in alternative embodiments.

Next, process 2550 proceeds to act 2556 that includes identifying, using the DNA sequencing data (generated at act 2554), a subset of a plurality of candidate methylation sites for which DNA sequencing data indicates DNA methylation status. After, process 2550 proceeds to act 2558 that includes generating a sparse DNA methylation profile using the DNA sequencing data. After, process 2550 proceeds to act 2560 that includes identifying the type of tumor in the patient by processing the sparse DNA methylation profile using a trained neural network model to obtain output indicative of the type of tumor in the patient. The output also indicates a confidence (e.g. a likelihood or probability) in the indicated tumor type. The acts 2556, 2558, and 2560 may be performed in any suitable way described herein including with reference to acts 2504, 2506, and 2508 of process 2500.

Next process 2550 proceeds to decision block 2562, where it is determined whether the confidence associated with the output obtained at act 2560 is above a predetermined threshold. Examples of such thresholds are provided herein. When it is determined that the confidence associated with the output obtained at act 2560 is above the predetermined threshold, the identified tumor type is output. On the other hand, when it is determined that the confidence associated with the output obtained at act 2560 is not above the predetermined threshold, the process 2550 returns to act 2554 where additional DNA sequence data may be generated through further sequencing of the biological sample obtained from a patient. In this way, the process 2550 may continue either until sufficient DNA sequence data has been obtained through sequencing of the biological sample to allow the trained neural network to identify the tumor type with sufficient confidence (e.g., until the confidence associated with the output of the neural network exceeds the predetermined threshold) or until an overall time limit (e.g., the amount of time this process can take during surgery) has been reached, whichever is smaller.

### EXAMPLES

### Example 1: DNA Modification Based Deep Learning (DL)-Classifier (Sturgeon) Model Development

### Data Simulation

Short duration Nanopore sequencing runs are sparse in an unpredictable manner. To be able to apply a model on such type of data, Nanopore simulated runs were generated and applied to the Illumina Infinium methylation data to sparsify them. First Illumina Infinium data was binarized to simulate the <1× genome coverage expected from nanopore sequencing using a beta score threshold of >0.6 to call methylation.

The objective of the data simulations is to generate a realistic set of sequenced CpG methylation sites. In order to do so, the simulation has several main parameters: the number of reads sequenced, the length of the reads, and the methylation calling error rate. Given a desired number of reads, a random sample would be taken from a read length distribution obtained from previously generated nanopore sequencing runs (only read length is sampled, not mapping location). Then, each read would be randomly assigned a start position across the genome by randomly sampling a value between 0 and ˜3 billion. This is done to give a proportional chance based on chromosome length. Each read was randomly assigned forward or reverse directions. Reads that, given their length and sampled location, map to two consecutive chromosomes were clipped accordingly to their direction. Using these parameters, CpG methylation status from a reference Illumina array sample were sampled. Given this set of reads, it was checked which CpG methylation sites would have been sequenced and subset a given microarray sample. Secondly 10% of the measured sites were randomly selected and their methylation status flipped to take into account the methylation calling error rate expected in nanopore methylation calling.

Training Set Generation Based on the procedure above, 100 k runs used for training were simulated. Additionally, 500 independent runs used for validation were simulated; and another 500 independent runs used for testing the model. Runs are simulated to include the expected number of reads between a 5 minute to a 60 minute sequencing run, in 5 minute intervals.

### Cross-Validation

To assess the performance of the model the reference cohort (Capper et al) dataset was split in four equally sized folds. The distribution of the samples across folds was randomized but class distributions were kept. Two folds for training were used, one for validation to assess the best model state and score thresholds, and one for testing to assess the final model performance. A total of four models were trained by rotating the folds; in this manner the model could be tested on the complete Heidelberg cohort while maintaining proper cross-validation practices.

### Model Training

A neural network to classify CNS tumor types was trained. The architecture of the neural network is that shown in FIG. 23B and is composed of three linear layers. The first two layers have 256 and 128 channels respectively, and are followed by a sigmoid activation. The last linear layer has as many channels as classes to be predicted, and is followed by a softmax activation. During training a single cross-entropy loss, as a classification loss, was used. The neural network for a total of 1000 epochs was trained with a batch size of 64 and a dropout rate of 0.5 between layers. The AdamW optimizer (Loshchilov n.d.) with a starting learning rate of 1e-5 was used that was increased linearly for 1000 training steps until 1e-3; afterwards, it was decreased using a cosine function until it reached 1e-5 at the last training step. Other parameters of the optimizer were: beta1=0.9, beta2=0.999, epsilon=1e-8 and weight_decay=0.0005. During training the amount of samples per class are balanced by upsampling the number of samples of each class to the class with the most samples. In this manner, class imbalance issues were avoided in the training set. Upsampling is done by simply applying additional simulations to the samples in a particular class. Furthermore, the applied simulations were balanced regarding the time component by ensuring that all classes have a balanced distribution of simulation times

The performance of the model was evaluated every 500 training steps using batches of the samples in the validation fold. These validation samples were also upsampled in the same manner as the training samples but used their independently simulated runs.

After every epoch of training, the performance of the model was evaluated on the train set to adjust the sampling. This leads to increasing the amount of samples and timepoints where the model is doing worse, while keeping the total amount of samples per epoch equal. The adjustment of the sampling is done by counting the percentage of incorrect predictions for every class and time point interval (5 minutes) and adding a correcting factor (for example, 0.5). The fraction of each class and time point was calculated by dividing the incorrect prediction percentages by the sum of all percentages. And then calculate the amount of samples for every class and time point based on these fractions and the total amount of samples per epoch.

### Model Evaluation

Model performance and score thresholding was done on the state of the model with the highest sensitivity. This sensitivity was calculated every 500 training steps using 25 batches of samples in the validation fold. The final performance of the model was assessed on the left-out test fold samples. Each sample was simulated on all 500 Nanopore simulation test runs on all 5 minute intervals. Sensitivity, precision and f1-scores were reported for each class individually across all time intervals, as well as average metrics across classes.

### Model Inference

Because of the cross-validation scheme, 4 models were trained independently and validated. During inference, all 4 models in parallel were used and the final prediction as the average weighted score predicted across all 4 models was reported. The weights of the average are calculated based on each individual model performance on the test set on each time interval.

### Sample Prep

DNA samples were obtained from the PMC biobank and stored at 4 degrees Celcius until processing. Library prep was performed using the nanopore rapid barcoding kit (SQK-RBK004), while multiplexing up to 4 samples per MinION flowcell (R9.4). At least 200.000 reads per sample are preferably read. Reads were split into batches of 5000 reads to simulate the yield of 5 minutes of sequencing. Different sequencing times were simulated by combining multiple batches.

### Data Processing

Samples were processed using Megalodon v2.5.0 basecalled using Guppy Version 5 and mapped to the CHM13 V1.1 reference genome using minimap2. Methylation calling was performed using Rerio model res_dna_r941_min_modbases_5mC_CpG_v001. When multiple reads covered a single position, a majority vote was used to determine the final call, when an equal number of reads called methylated and unmethylated, the call was discarded from further analysis. The trained classifiers were applied to the resulting list of methylation calls.

### REFERENCES

Capper, David, David T. W. Jones, Martin Sill, Volker Hovestadt, Daniel Schrimpf, Dominik Sturm, Christian Koelsche, et al. 2018. “DNA Methylation-Based Classification of Central Nervous System Tumors.” Nature 555 (7697): 469-74.
Euskirchen, P. et al. Same-day genomic and epigenomic diagnosis of brain tumors using real-time nanopore sequencing. Acta Neuropathol. 134, 691-703 (2017).
Loshchilov, Ilya. n.d. AdamW-and-SGDW: Decoupled Weight Decay Regularization (ICLR 2019). Github. Accessed Aug. 5, 2022. https://github.com/loshchil/AdamW-and-SGDW.
Meissner, Alexander, Tarjei S. Mikkelsen, Hongcang Gu, Marius Wernig, Jacob Hanna, Andrey Sivachenko, Xiaolan Zhang, et al. 2008a. “Genome-Scale DNA Methylation Maps of Pluripotent and Differentiated Cells.” Nature 454 (7205): 766-70. 2008b. “Genome-Scale DNA Methylation Maps of Pluripotent and Differentiated Cells.” Nature 454 (7205): 766-70.

### Example 2—Classification of Brain Cancer Samples

As a proof of concept classifiers for CNS subtype classification were developed, based on publicly available data provided in Capper et al. This dataset contains correctly labeled methylation profiles consisting of 450,000 sites from 2801 CNS tumor and control samples. When performing intraoperative sequencing, only a fraction of the 450,000 sites in the reference panel is expected to be covered.

Sturgeon can be designed to classify brain cancer samples during surgery using nanopore sequencing, where the intended purpose is to classify the sample in real time as the sequence reads are being generated.
1. Reference data: GSE109381 dataset contains DNA methylation profiles derived from 91 types of CNS cancer and 2801 samples using the Infinium HumanMethylation450 BeadChip array and thus contain continuous values for ˜450.000 CpG sites in the genome.
2. Training data:
Sturgeon is trained for nanopore long read sequencing:
   Read length (average ˜6 kilobases) is modeled according to a whole-genome MinION Nanopore sequencing run.
   Number of reads per unit of time is modeled according to a whole-genome MinION Nanopore sequencing run.
   Error rate: at least 10%.
   Read mapping is uniform across the genome.
   Methylation values are binarized.
3. Training: Sturgeon is trained to classify the 91 classes of brain cancer as defined in the GSE109381 dataset using 100.000 class-balanced simulated training profiles.
4. Inference data: The inference data consists of shallow nanopore long read sequencing of brain cancer biopsies with unknown class. The number of obtained reads, and thus the coverage of methylation sites, increases over time. Sturgeon is applied at a 5 minute interval (corresponding to approximately batches of 4000 reads) until a high confidence classification is obtained.

### Example 3—Classification of Melanoma Samples

Illumina high throughput sequencing is widely available and allows for multiplexing of samples. Detecting the difference between melanoma and healthy skin can possibly be done using methylation profiles. Using Sturgeon, a method is plausible where hundreds of samples can be tested in parallel at a very low cost per sample.

### 1. Reference Data:

Deep whole genome bisulfite sequencing data containing genome-wide methylation frequencies for 10 melanoma biopsies and 10 healthy skin biopsies.

### 2. Training Data:

Sturgeon training data is generated for shallow bisulfite sequencing:
Classes (melanoma and healthy skin) are upsampled to 50.000 training samples each.
Reads are 200 bp in length
1 million random reads are generated per simulated training sample, randomly positioned in the genome.
Methylation calls are binary.
the expected error rate: 3%.

Training: A classifier is trained on 50.000 simulated melanoma samples and 50.000 simulated healthy skin samples to solve a two-class problem: detect melanoma versus healthy skin.

Inference data: About 400 Shallow whole genome bisulfite sequencing profiles from melanoma-suspected skin biopsies. Samples are sequenced in a single Illumina nextseq sequencing run yielding approximately 1 million reads per sample.

### Example 4: Sturgeon Achieves Accurate Classification in >90% of the Test Cases

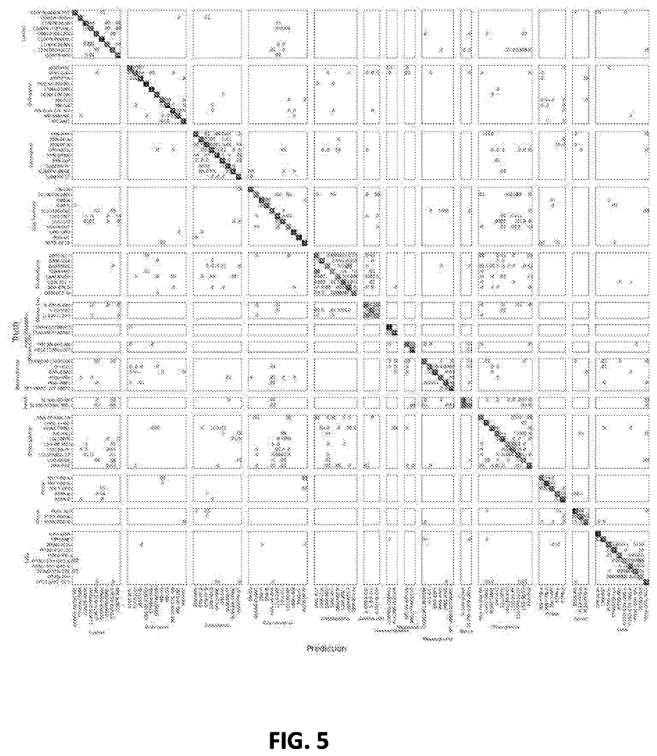

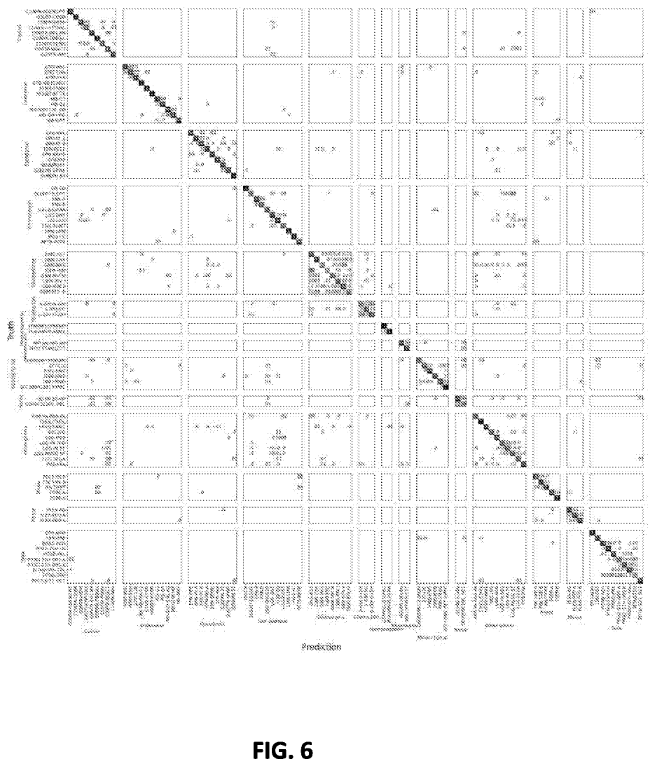

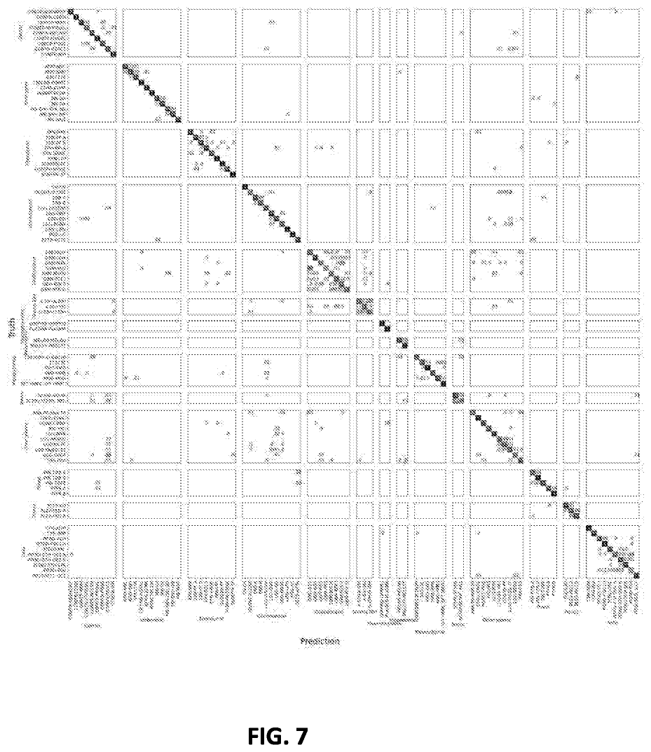

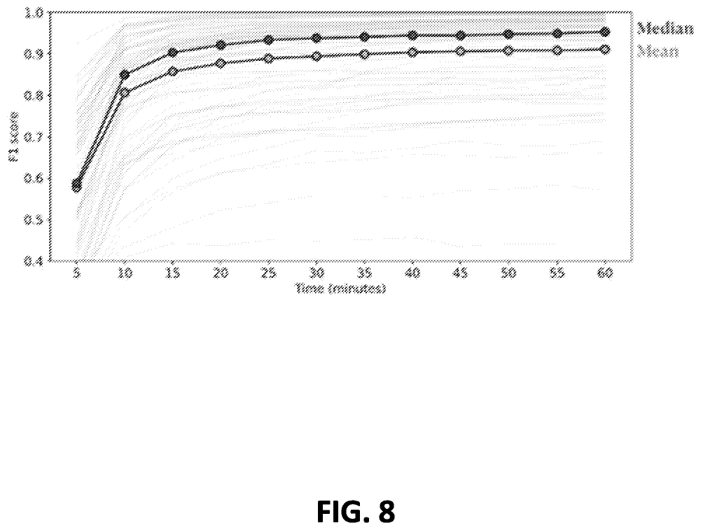

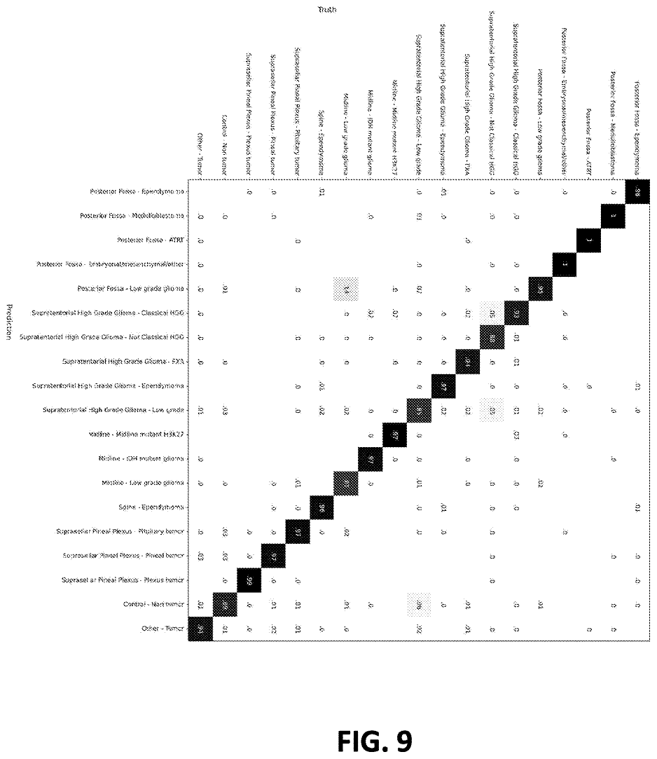

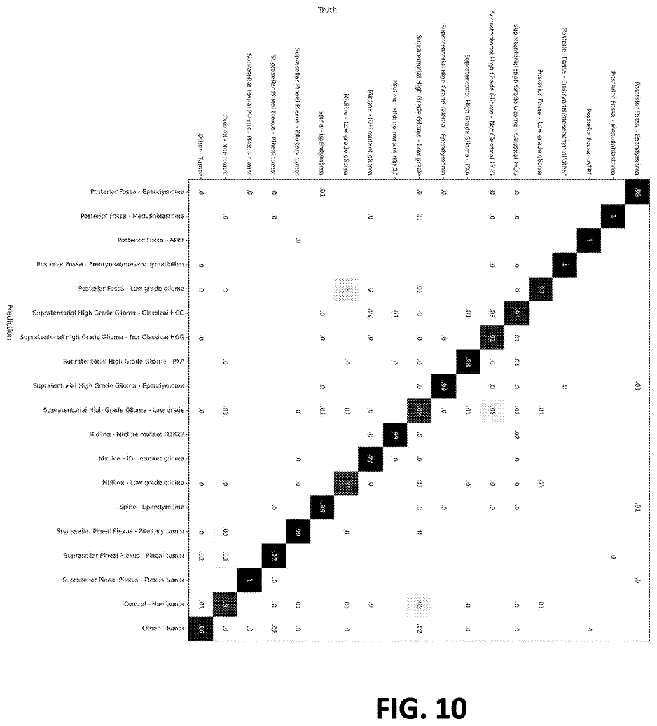

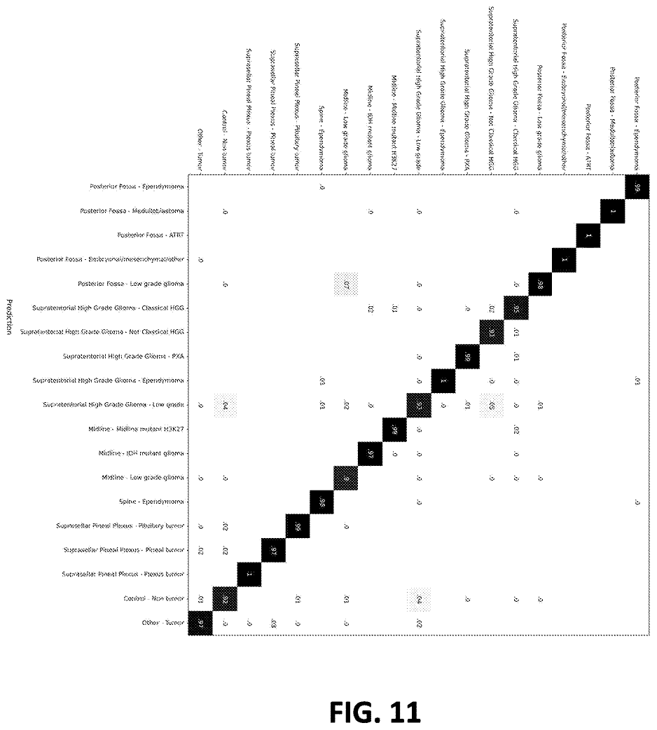

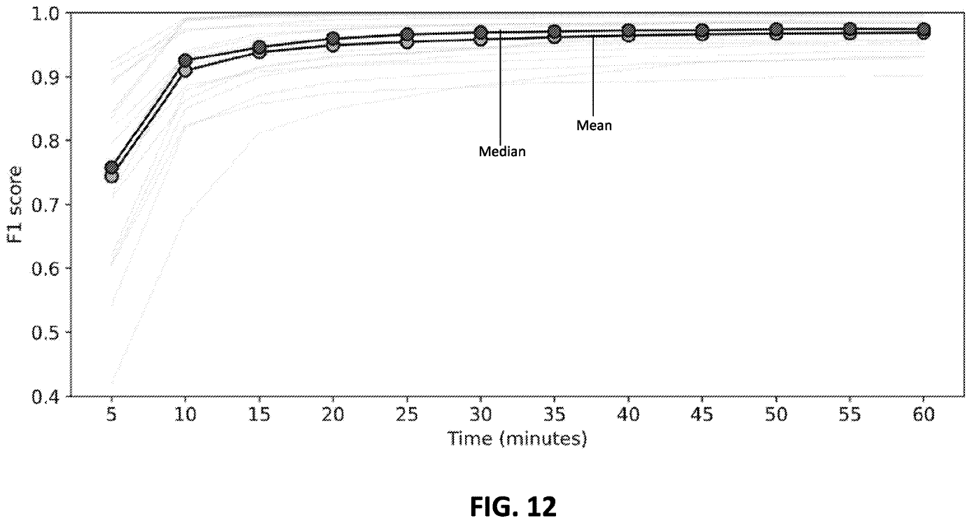

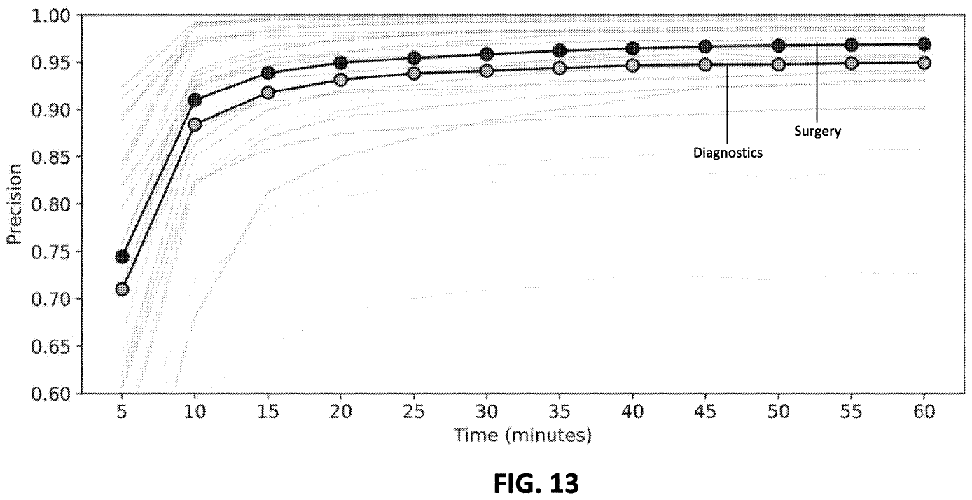

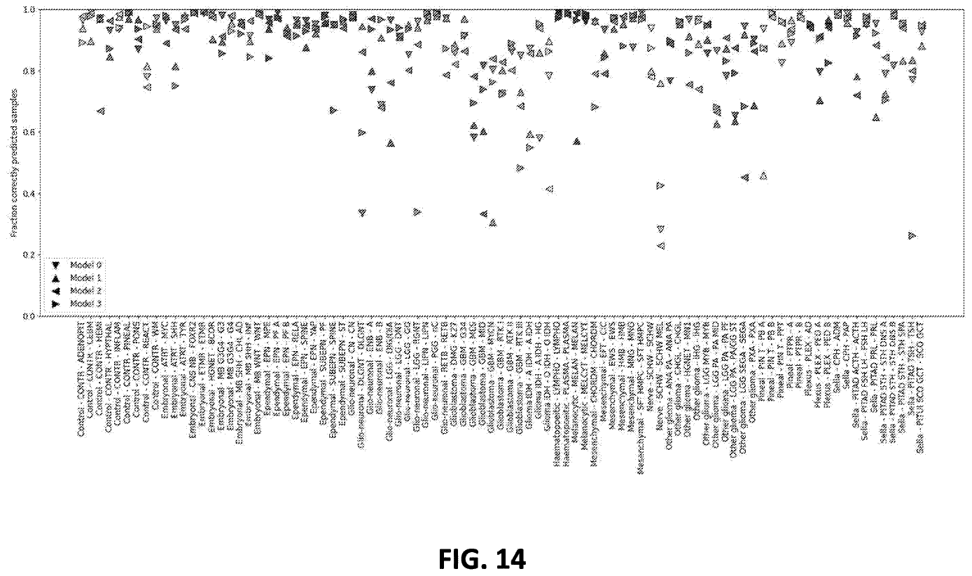

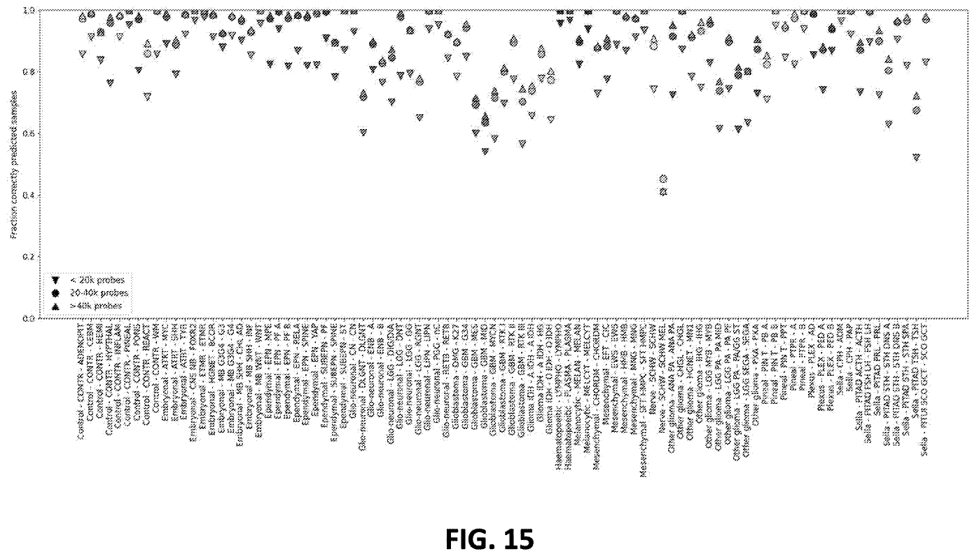

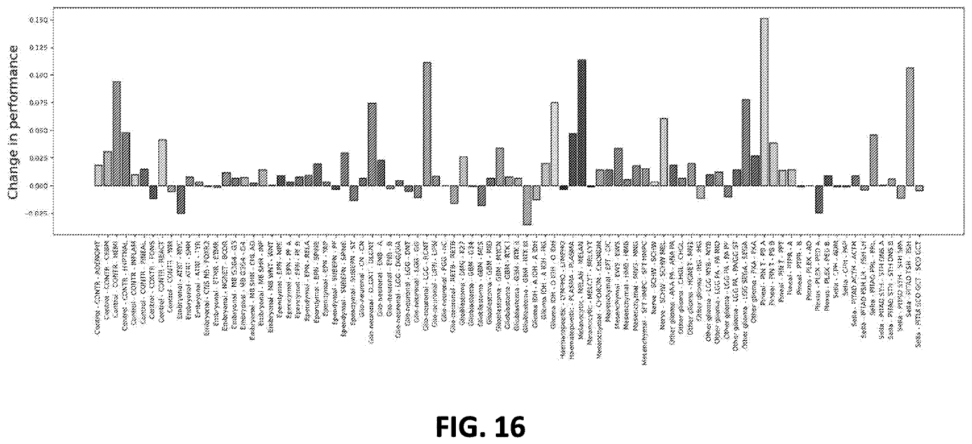

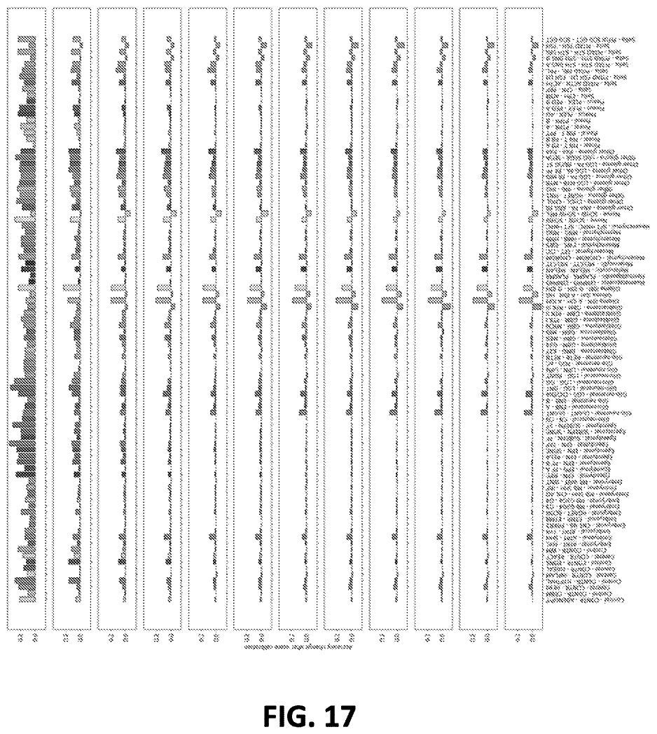

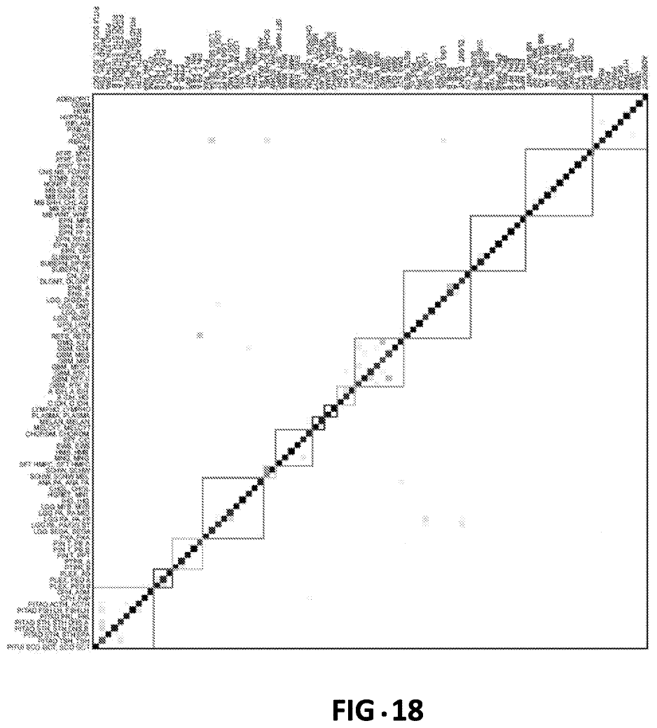

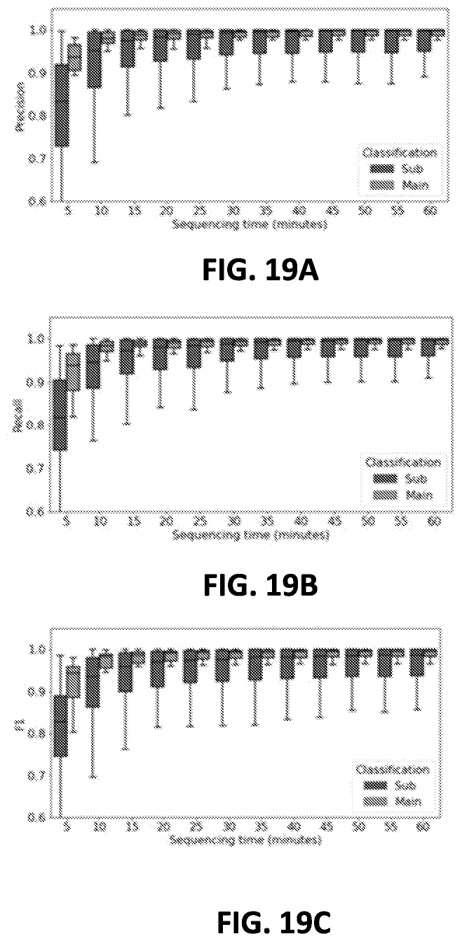

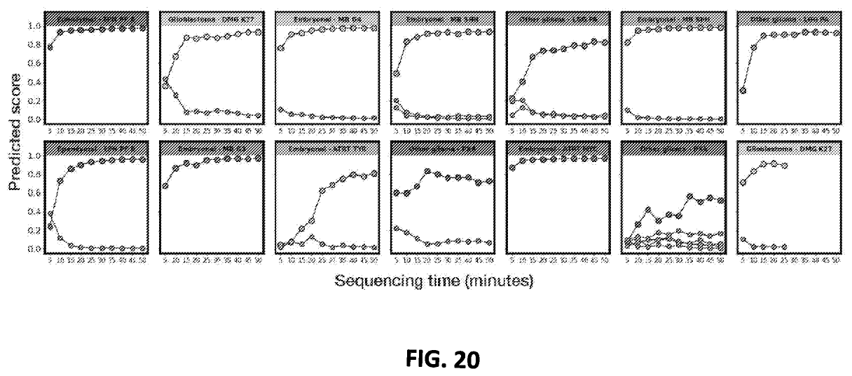

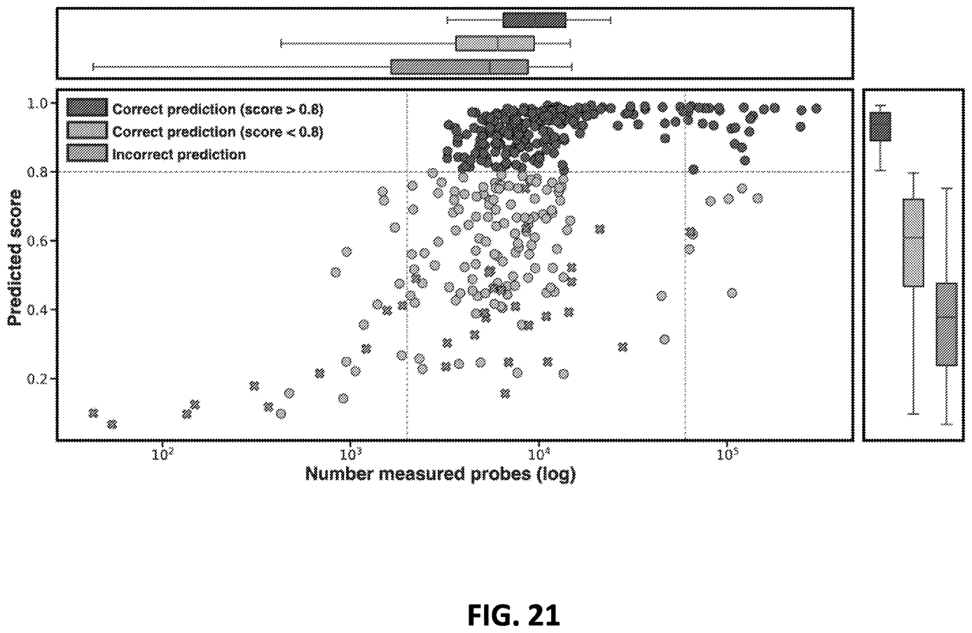

In one example the DNA modification based deep learning (DL)-classifier was trained on 75% of the simulated profiles until performance was optimal. Then the classifier on the remaining 25%. The confusion matrix (FIG. 5-7) demonstrates that test performance is very high, with an accurate classification in >90% of the test cases (Table 1). Moreover, for the classes with slightly higher error, most often confusions are made with very similar CNS subtypes. These results demonstrate that Sturgeon classifies severely downsampled 450K profiles at a performance level similar to that originally published by Capper et al.

TABLE 1
Classifier performance at different levels of sparsity.

| Data lost | Probes covered | Recall |
|---|---|---|
| 98% | 8000 | 86% |
| 96.5% | 16000 | 90% |
| 95% | 24000 | 92% |

### Example 5: Sturgeon is Able to Correctly Diagnose in 14 Cases

To establish that the herein described classifier can also classify on real Nanopore data, 14 patient DNA samples were sequenced, selected to be representative of the clinical population in the Maxima center. Each sample was sequenced to a depth of at least 200.000 reads on a MinION flowcell. The Sturgeon classifier was applied to all the sequence reads collected up to a simulated timepoint (FIG. 22). In all cases the correct diagnosis was reached within 15 minutes (Table 2 lists results of all 14 cases). Based on these results it was contended that Sturgeon achieves accuracies and turnaround times compatible with intraoperative sequencing and, when further developed, has a real opportunity to improve patient care. In other words, by using the computer-implemented method and classifier as disclosed herein, a conclusive diagnosis was reached in 14/14 cases within 15 minutes of sequencing.

TABLE 2
Preliminary results of the first 14 samples (see also FIG. 22 - classifier score over time).
The classifier was run on a downsampled batch of each sample. The resulting diagnosis is
shown alongside the clinical diagnosis. A sequencing run was simulated by adding increments
of 5000 reads, the approximate yield of 5 minutes of sequencing. Shown is the number of
methylation probe sites and approximate time needed to reach the final diagnosis.

| | | | | Sites | |
|---|---|---|---|---|---|
| | | | | covered at | Sequencing |
| | sequenced | | | first correct | g time |
| Pat # | reads | classifier outcome | pathology diagnosis | diagnosis | (minutes) |
| 1 | 400.000 | Embryonal Ependymoma PF A | Ependymoma PF A | 4574 | 5 |
| 2 | 400.000 | Diffuse Midline Glioma | Diffuse Midline Glioma | 6474 | 10 |
| | | H3-K27 | H3-K27 | | |
| 3 | 200.000 | Embryonal medulloblastoma | Medulloblastoma Group 4 | 3040 | 5 |
| | | Group 4 | (subtype VIII) | | |
| 4 | 400.000 | Medulloblastoma SHH | Medulloblastoma SHH | 10647 | 15 |
| 5 | 450.000 | Pilocytic astrocytoma Posterior | Pilocytic astrocytoma | 13545 | 15 |
| | | fossa or anaplastic Pilocytic | | | |
| | | astrocytoma | | | |
| 6 | 270.000 | Medulloblastoma SHH | Medulloblastoma SHH | 7543 | 10 |
| 7 | 200.000 | Pilocytic Astrocytoma | Pylocitic astrocytoma | 8087 | 10 |
| 8 | 240.000 | Ependymoma PF B | Ependymoma PF B | 8286 | 10 |
| 9 | 306.000 | Medulloblastoma Group 3 | Medulloblastoma Group 3 | 2937 | 5 |
| 10 | 200.000 | Atypical teratoid/rhabdoid | Atypical teratoid/rhabdoid | 1404 | 15 |
| | | tumor, subclass TYR | tumor, subclass TYR | | |
| 11 | 320.000 | Pleomorphic | Pleomorphic | 4103 | 5 |
| | | xanthoastrocytoma | xanthoastrocytoma | | |
| 12 | 290000 | Atypical teratoid/rhabdoid | Atypical teratoid/rhabdoid | 4865 | 5 |
| | | tumor, subclass MYC | tumor, subclass MYC | | |
| 13 | 315000 | Pleomorphic | Pleomorphic | 6448 | 10 |
| | | xanthoastrocytoma | xanthoastrocytoma | | |
| 14 | 20.000* | Diffuse Midline Glioma | Diffuse Midline Glioma | 5271 | 10 |
| | | H3-K27 | H3-K27 | | |

*sample 14 was under sequenced.

### Example 6: Ultra-Fast Deep-Learned CNS Tumor Classification During Surgery

Central nervous system (CNS) tumors represent one of the most lethal cancer types, particularly among children¹. Primary treatment includes neurosurgical resection of the tumor, in which a delicate balance must be struck between maximizing the extent of resection and minimizing risk of neurological damage and comorbidity^{2,3}. However, limited knowledge of the precise tumor type is available prior to surgery. Current standard practice relies on preoperative imaging and intraoperative histological analysis, but these are not always conclusive and occasionally wrong. Using rapid nanopore sequencing, a sparse methylation profile can be obtained during surgery⁴. To enable molecular subclassification of CNS tumors on such profiles, Sturgeon was developed: a patient-agnostic transfer-learned neural network that delivers an accurate diagnosis within 40 minutes after starting sequencing in 45 out of 50 retrospectively sequenced samples (abstaining from diagnosis in the other five). Furthermore, the applicability in real time was demonstrated during 25 surgeries, achieving a diagnostic turnaround time of less than 90 minutes. Of these, 18 (72%) diagnoses were correct and 7 did not reach the required confidence threshold. It was concluded that machine-learned diagnosis based on low-cost intraoperative sequencing can assist neurosurgical decision making, potentially preventing neurological comorbidity and avoiding additional surgeries.

Most commonly, the first line treatment of CNS tumors is neurosurgical resection of the tumor. An important factor in determining if the risk of a more aggressive resection is acceptable is the tumor type. For instance, diffuse midline gliomas with a specific Histone 3 (H3K27) mutation are considered incurable, indicating that surgery should primarily be aimed at acquisition of tumor tissue for diagnosis and preserving quality of life, rather than attempting total resection⁵. Likewise, medulloblastoma cases show limited prognostic improvement between near-total and total resection, indicating that also for these tumors, maximal resection is not necessarily preferable⁶. For other tumors however, radical resection is beneficial: in posterior fossa Ependymoma type A (PFE-A) and atypical teratoid rhabdoid tumor (ATRT) cases a strategy of aiming at a gross total resection (GTR) should be followed since this is an important prognostic factor⁷⁻¹⁰. Also in CNS tumors in adults the extent of resection matters: GTR has been reported to offer survival benefits for IDH-wildtype Glioblastoma of the RTK I and RTK II subtypes, but not for the Mesenchymal subtype¹¹. Similarly, in IDH-mutant Astrocytoma overall survival is negatively impacted when GTR is not achieved¹². The neurosurgical strategy thus depends on a precise and reliable diagnosis of the tumor.

Altered genome-wide DNA methylation patterns are highly distinctive features of neoplasms, and the assessment of DNA methylation can reveal information about the origin and prognosis of a tumor¹³⁻¹⁶. Using machine learning approaches, in particular random forest classification, high-dimensional CpG methylation profiles can be accurately assigned to a specific CNS subtype^{14,15}. Methylation arrays^{17,18}, in combination with the algorithm described by Capper et al., are widely used in routine diagnostic practice. However, the turnaround time (TAT) for obtaining array-based methylation profiles is in the order of several days and therefore incompatible with an intraoperative setting.

Current practice consists of preoperative imaging and intraoperative diagnosis achieved by rapid histological assessment of frozen tumor sections. However, this does not always result in a clear diagnosis, and sometimes the provisional frozen-section diagnosis is revised based on postoperative tissue-based diagnostics. As a result, some patients require a second surgery, while others could in hindsight have been operated less radically.

Nanopore DNA sequencing recently emerged as a method that enables ultrarapid sequencing-based diagnosis^{19,20}. Major advantages of nanopore sequencing include its low setup cost, small form-factor and instant data-availability. In addition, nanopore sequencing allows direct measurement of methylated cytosines and a significant reduction in sample preparation times²¹. Thus a tissue sample can be sent for sequencing in the early stages of surgery to obtain a molecular diagnosis in time to affect and shape the neurosurgical strategy⁴. A major challenge of this application is that in such a short time, only very sparse methylation profiles can be generated. Moreover it is a priori unknown which CpG sites will be covered.

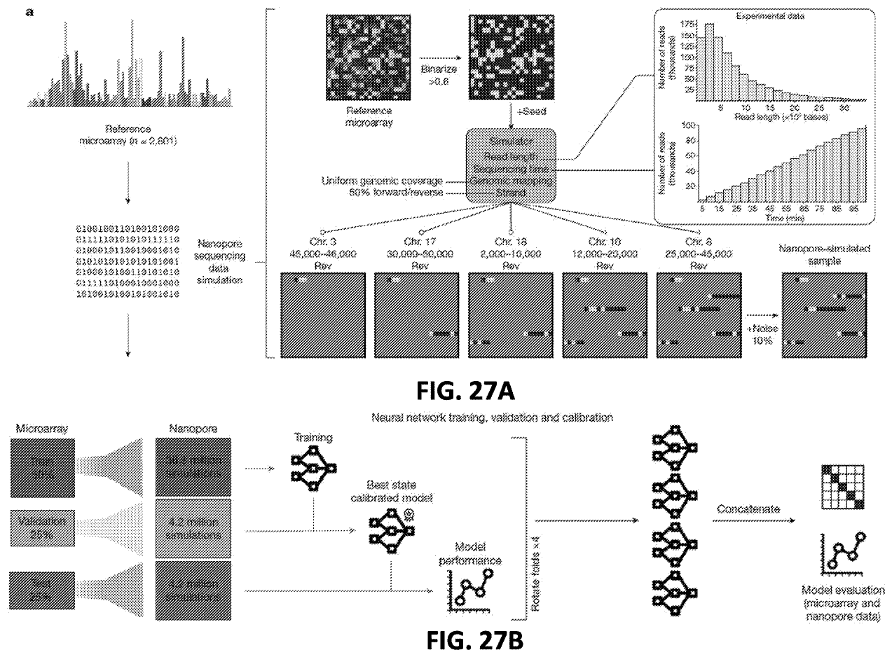

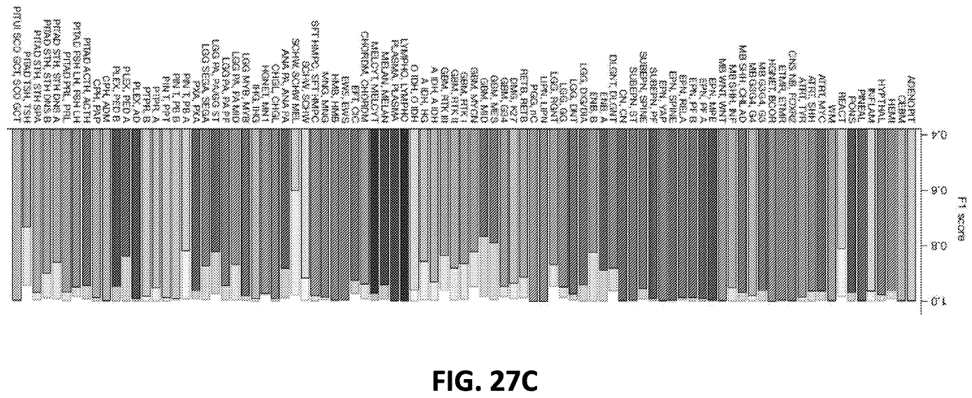

To enable tumor classification in an intraoperative setting, Sturgeon was developed: a neural network classifier that is patient agnostic and optimally tuned to deal with sparse data. In the Sturgeon approach, extensive computational resources are allocated to train and validate complex neural networks prior to surgery. This is a major advantage over existing classification algorithms that rely on patient-specific model training during surgery⁴. The final models are trained on 14 million and validated on 4 million simulated nanopore runs, respectively (FIGS. 27A-C). Moreover, this allows us to extensively validate and calibrate the models prior to applying them. The resulting Sturgeon model is portable and only takes a few seconds to run on a laptop CPU.

As a proof of concept, Sturgeon models were trained for CNS tumor classification, and retrospectively applied them on sparse nanopore sequencing data in 50 CNS tumor samples and 415 publicly available²² nanopore sequenced CNS samples. The model is able to correctly classify the vast majority of patients (45 out of 50) based on data equivalent to 20-40 minutes of sequencing, in line with a 90 minute time-window between obtaining tissue in the operating room and diagnosis. Finally, the ability of Sturgeon to influence surgical decision making by applying it in a realistic intraoperative setting for 25 CNS tumor resections was demonstrated.

### Neural Network Training Via Simulation

Within a TAT of 60-90 minutes only very limited nanopore sequencing data, in the order of 100-400 Megabases, can be generated. As a result, extremely sparse coverage across the genome is expected (e.g., covering 0.5-4% of the CpG sites in a 450K array). As it is a priori unknown which sites will be covered, this poses a significant challenge for the downstream machine learning model. Large, well-annotated nanopore-based methylation datasets are currently lacking and will take years to reach the comprehensiveness of the available array-based datasets. Therefore a simulation strategy that generates realistic training data from array-based methylation profiles was developed. Finally, effectively training neural network models requires orders of magnitude more training samples than the number of patient samples available. Sturgeon therefore employs a data augmentation approach to upsample the number of training samples available: thousands of unique shallow nanopore sequencing experiments are simulated from each methylation profile.

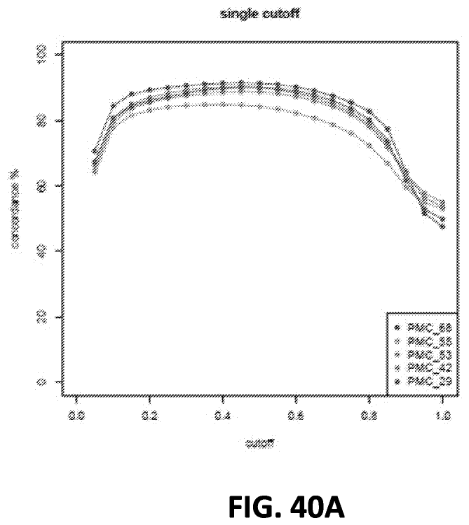

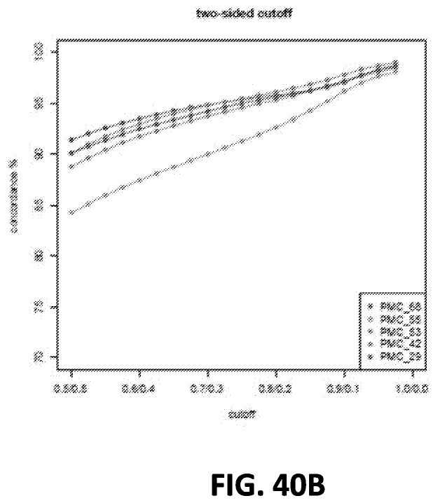

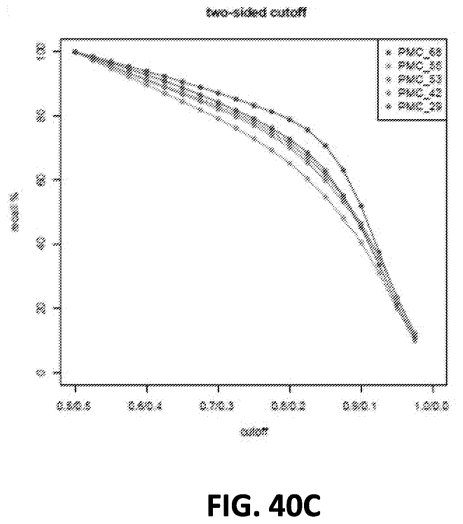

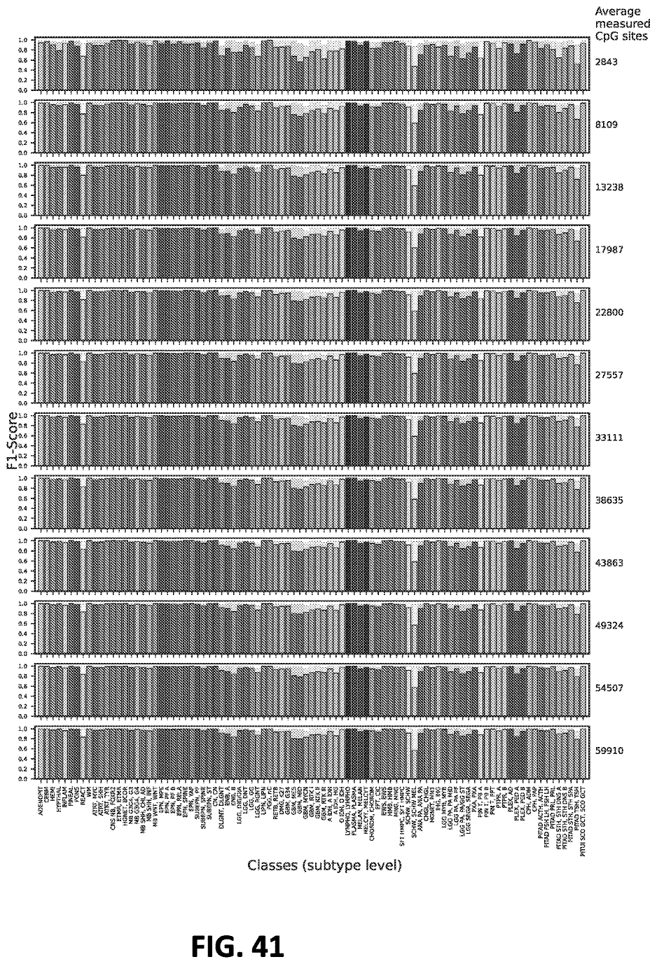

Sturgeon is designed to train a neural network on simulated nanopore sequencing runs. Here, the publicly available Infinium 450K profiles were used as reported in Capper et al¹⁴. This dataset contains 2,801 reference labeled methylation profiles from CNS tumor and control tissue samples. The simulation consists of the following components (FIG. 27A): (1) Binarization of the array beta values to either methylated or unmethylated state, to account for the limitation that in shallow sequencing the expected coverage is ≤1× for the vast majority of detected sites, precluding the ability to reflect heterogeneously methylated sites. (2) Non-uniform CpG site sampling to account for the fact that nanopore sequence reads are approximately 5 kilobases (kB) in length (assuming the rapid sample preparation methods used in an intraoperative setting). (3) Variable sampling of the number of CpG sites covered, to account for read accumulation as time progresses. (4) Random noise, to account for the expected discrepancy rate of 10-15% in nanopore methylation-aware sequencing compared to binarized methylation arrays, resulting from a combination of heterogeneous methylation states across alleles and cells, and due to methylation calling errors²³ (FIGS. 40A-40C).

The simulated nanopore sequencing data is used to train four neural networks (Methods, FIG. 23B). These neural networks are each trained, validated and calibrated independently (FIG. 27B). To this end, the Capper et al.¹⁴ reference dataset was split into 4 folds while keeping the original class distributions. Then, the two folds were used to train the submodel, one fold to determine the best performing state of the submodel and to perform score calibration and the final fold to evaluate the submodel's performance.

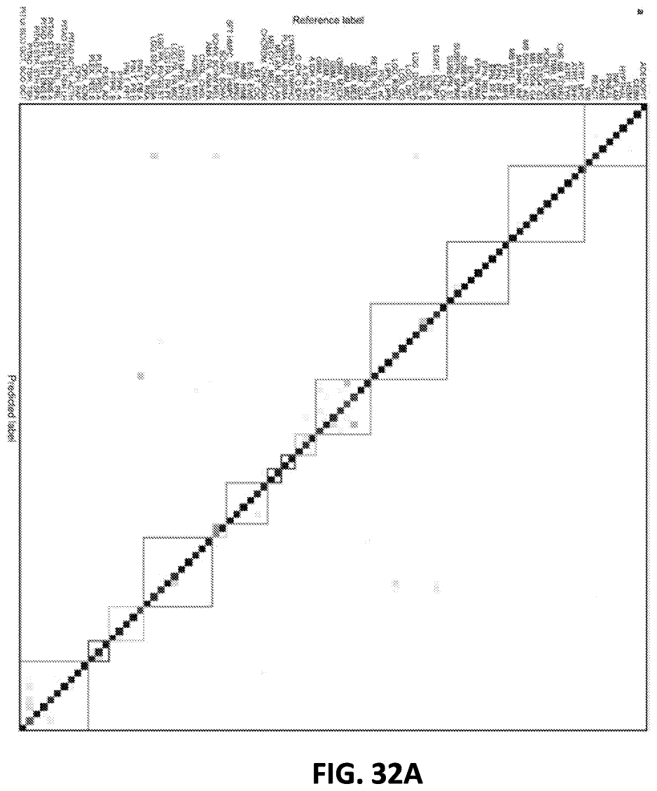

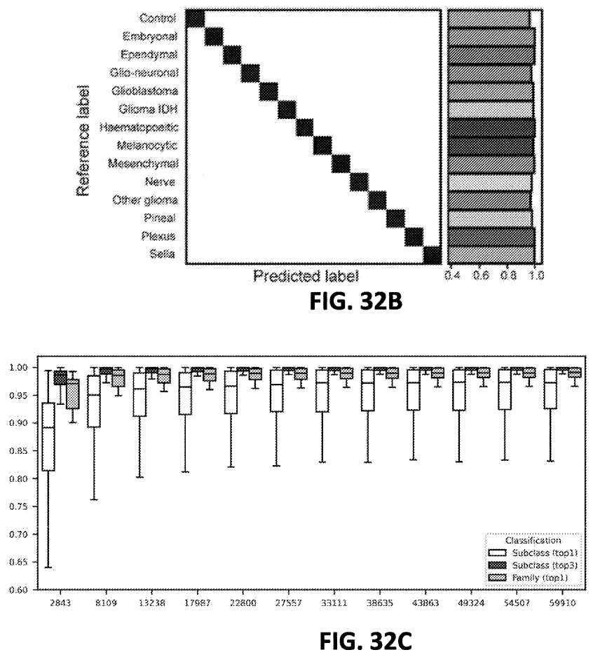

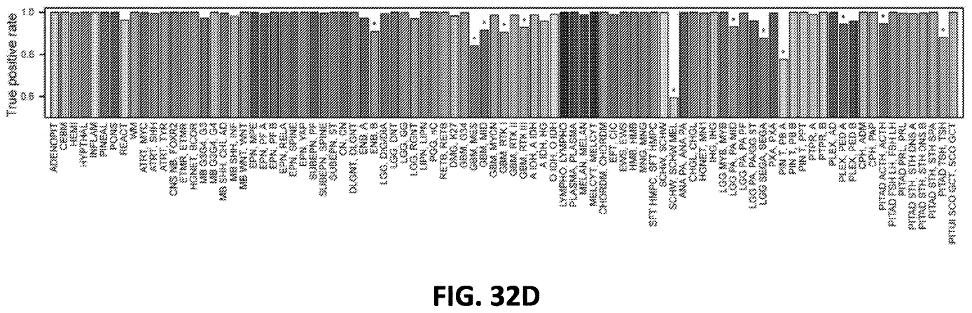

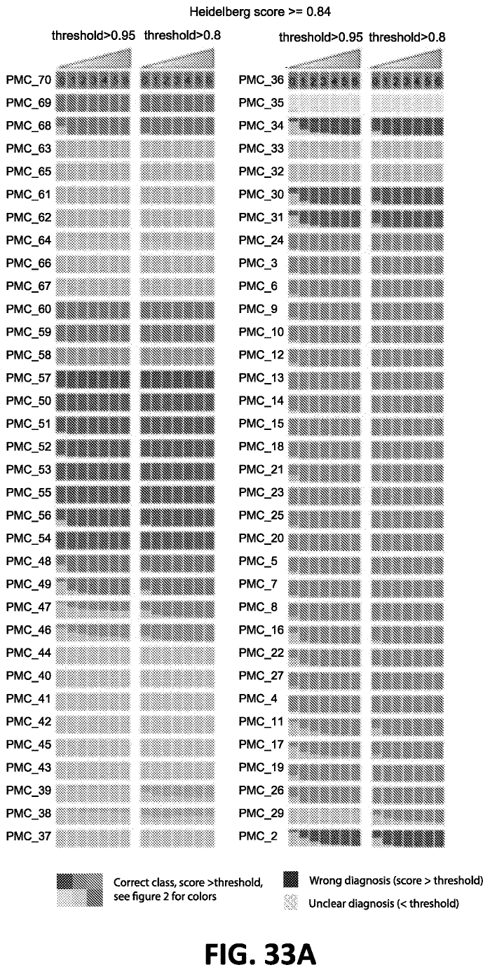

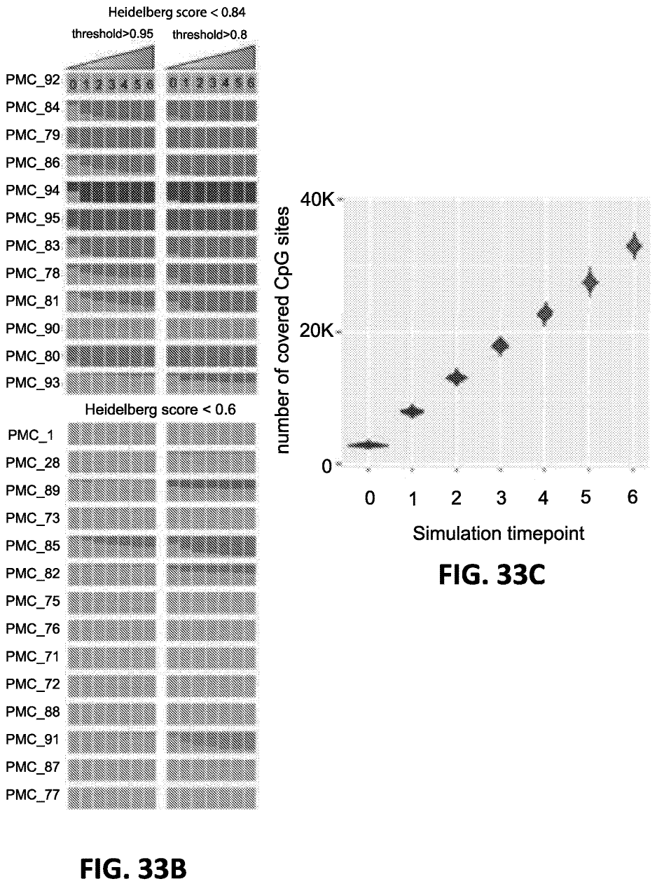

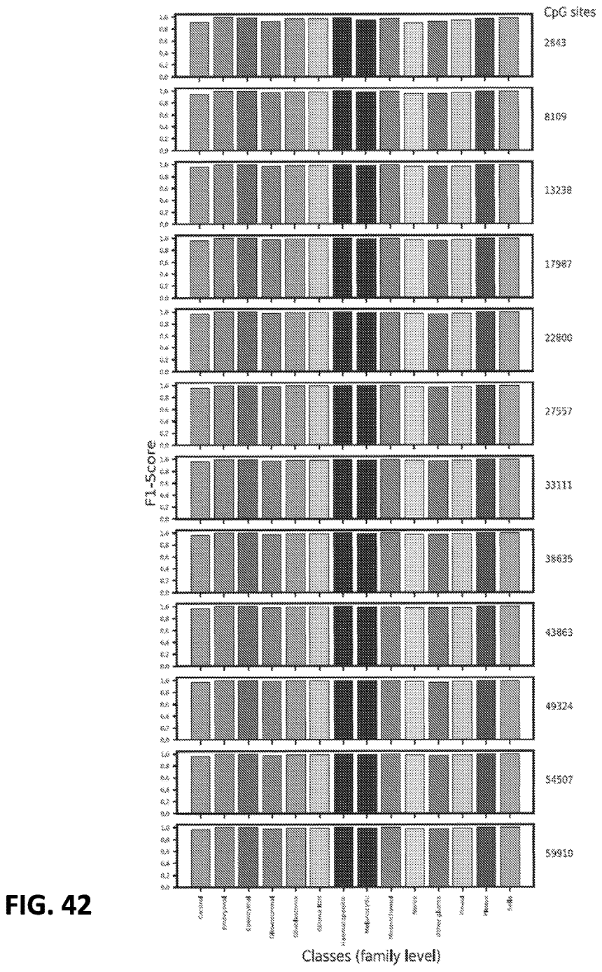

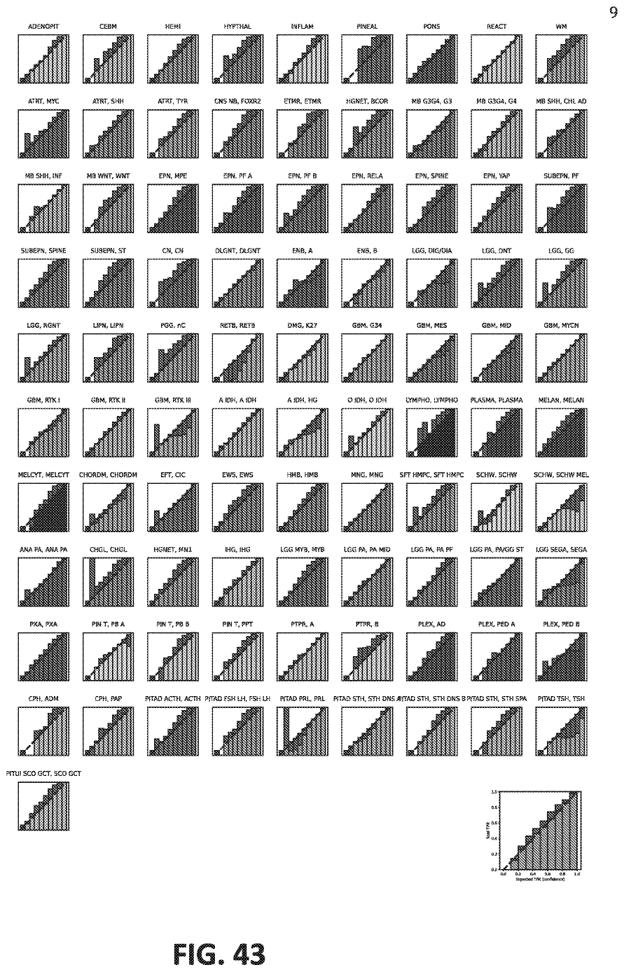

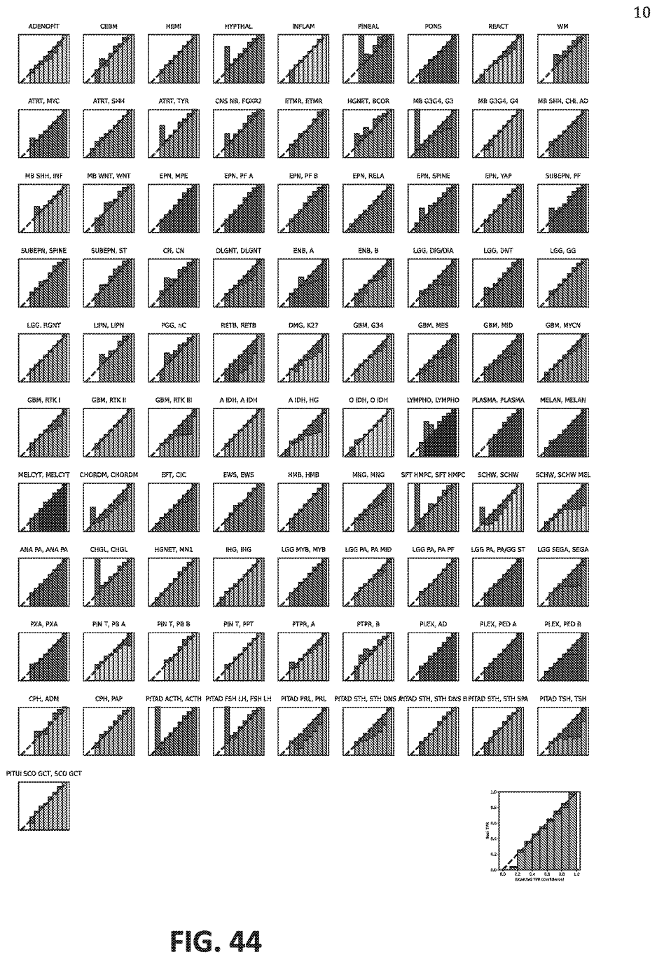

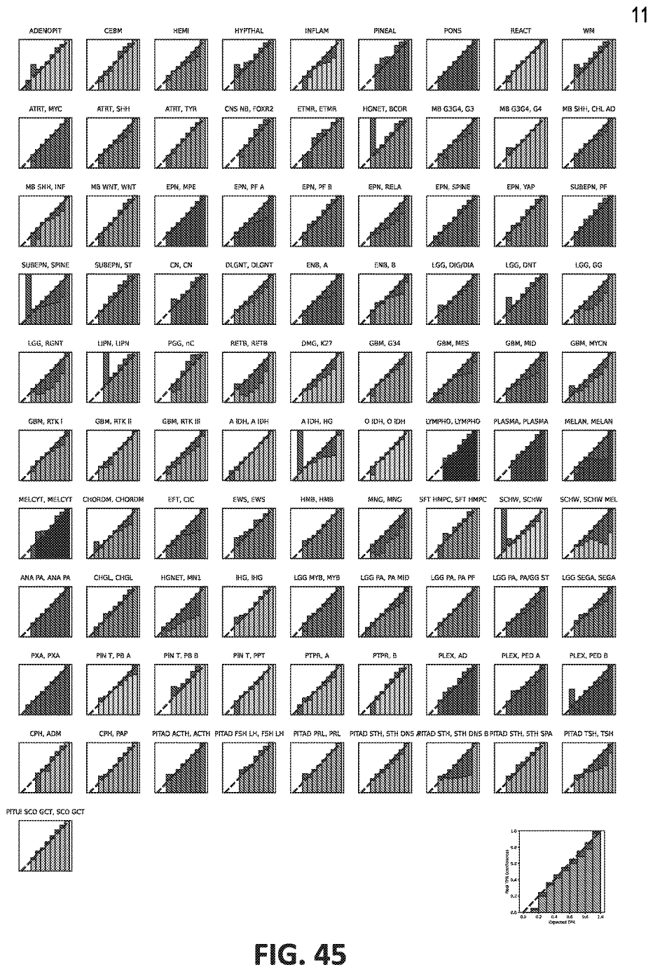

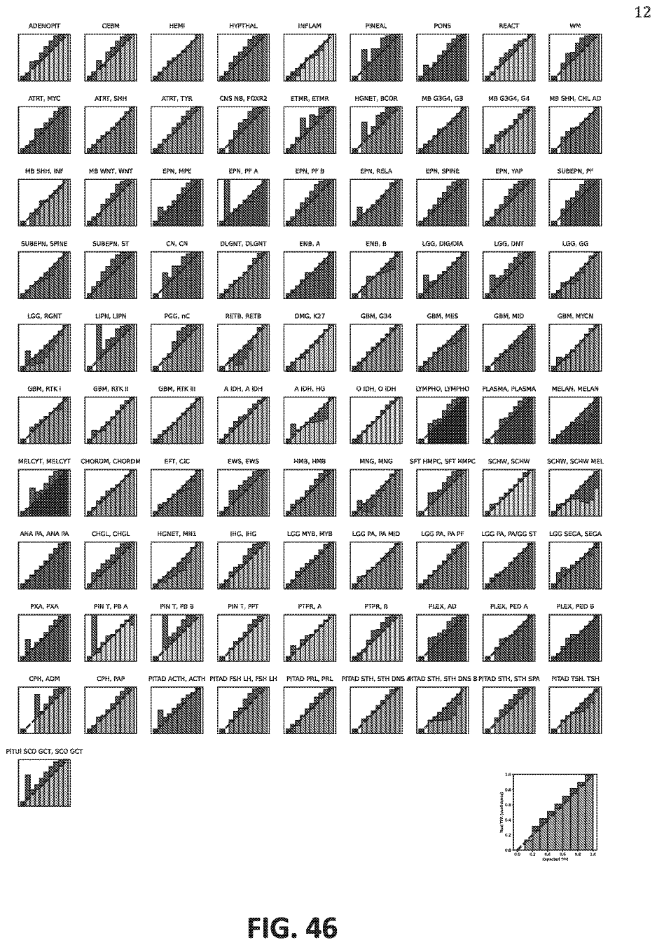

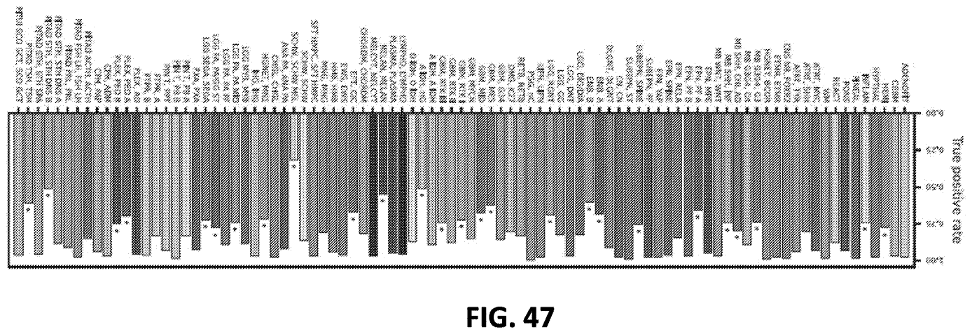

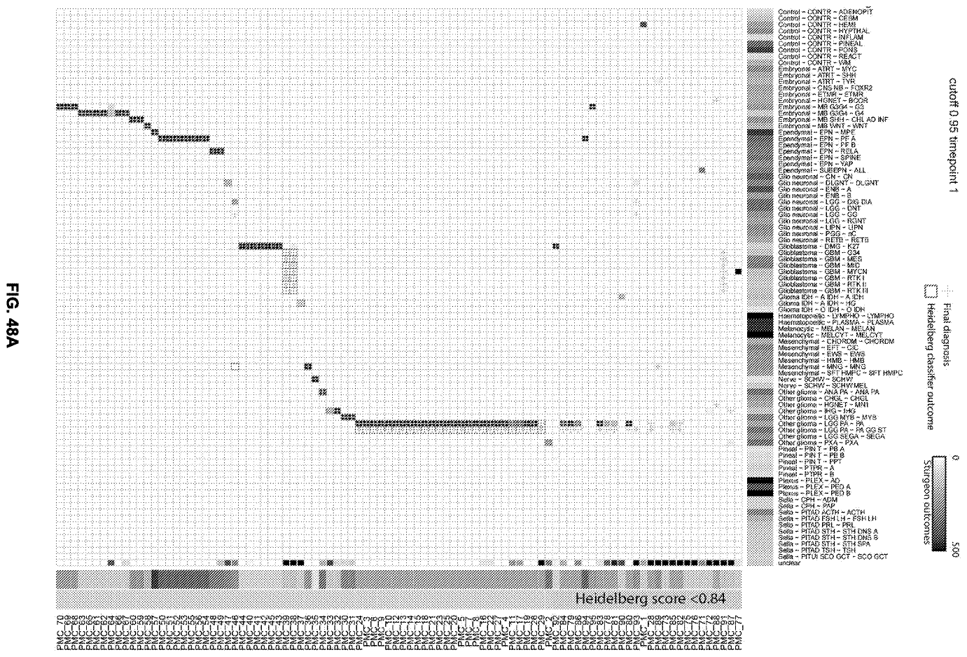

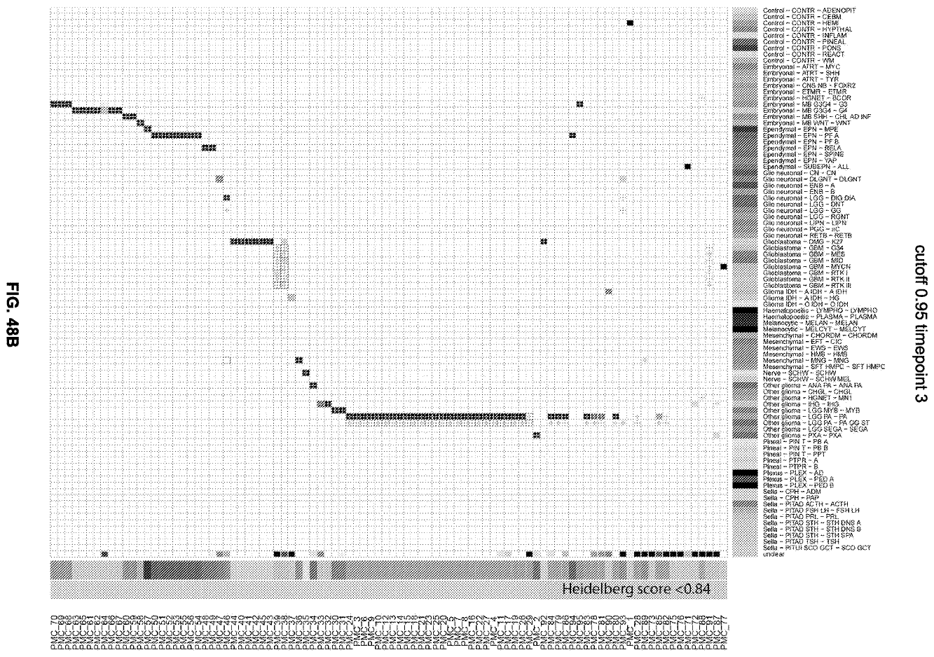

The Sturgeon submodel performance was evaluated on the hold-out test set. The submodels achieved a F1-score of 0.935 across all classes at the approximate equivalent of 40 minutes of sequencing (FIG. 27C). Specific classes show an increased error rate, as may be expected due to their biological similarity (e.g. Melanotic Schwannoma versus Schwannoma, different TSH secreting Pituitary Adenomas subtypes and highly similar Glioblastoma subclasses). When aggregating scores on the family level, performance is even higher with an average F1 score of 0.984 at the same sequencing time (FIG. 32B). As expected, Sturgeon's performance is directly correlated to the sequencing depth, and performance increases most drastically within the first 50 minutes of simulated sequencing with 0.6% to 4% of the 450K CpG sites covered (FIG. 32C, FIGS. 42-43). The classifier scores were then calibrated to ensure that, for example, a classifier score of 0.9 corresponds to the classifier being correct 90% of the time. For this purpose temperature scaling was applied²⁴. As a result of temperature scaling, the overall Expected Calibration Error (ECE), decreased from 0.025 to 0.002 in the test set (FIGS. 43-46). It was decided to conservatively use a cut-off score of 0.95 to confidently classify a sample. Using this cut-off, 80 out of the 91 classes in the test set have a True Positive Rate (TPR) higher than 0.95; With a less conservative threshold of 0.8, 26 out of 91 classes do not reach the expected 0.8 TPR (FIG. 32D, FIG. 47).

### Classification of Pediatric Array Data

The training dataset for Sturgeon consists of a varied patient population of different ages (mean age of 29; 36%<13 years of age). The first aim was to further validate Sturgeon's performance in a pediatric setting. For this purpose 94 EPIC profiles generated for patients that underwent a CNS tumor resection surgery in the Princess Maxima Center (PMC) for Pediatric Oncology were obtained. For each of these samples, the publicly available “Heidelberg classifier (v11b4)” was applied as part of the routine clinical care. This classifier can be considered an updated version of the Capper et al.¹⁴ classifier. The recommended cutoff for a clinical diagnosis²⁵ is 0.84, which it reached for the majority (N=68) of samples. Those classified below the 0.84 cutoff (N=26) are considered difficult to diagnose based on their methylation profile, which is likely to occur for uncommon tumor types that do not correspond to any of the previously annotated classes, tumors that occur in the context of a genetic tumor predisposition syndrome, heterogeneous samples or samples with a low tumor purity.

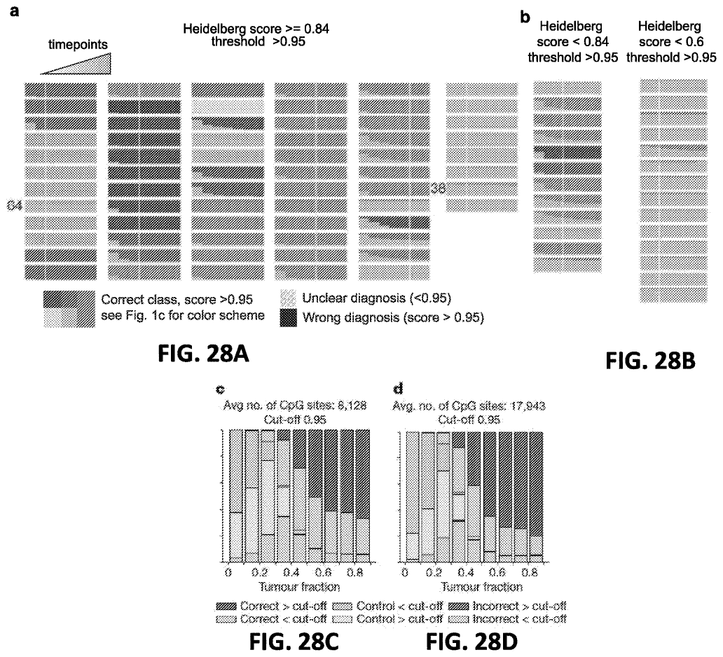

For each methylation profile 500 nanopore sequencing experiments were simulated at seven sequencing depths (FIGS. 28A-B) for a total of 332,500 simulated nanopore sequencing experiments; after which the Sturgeon classifier was applied (FIGS. 28A-B, 33A-33C).

For the clear diagnosis (Heidelberg score >0.84) cases, Sturgeon classified correctly (at the 0.8 threshold) in 95.3% (32,412/34,000 simulated samples) in as little as 25 minutes of simulated sequencing. At the conservative threshold of 0.95, still 86.2% (29,316/34,000) of simulated samples were correctly classified (FIG. 28A, second timepoint). At the same time point, only 2.7% and 13.8% of simulations did not reach a confidence score exceeding 0.8 and 0.95 respectively. Wrong diagnoses were called in 2.0% of simulations at the 0.8 threshold, and only 0.5% for the conservative 0.95 threshold. At 50 minutes of simulated sequencing (FIG. 28A, fourth timepoint) performance improved slightly, with 97.1% (33,020/34,000) of simulations reaching a correct diagnosis with confidence ≥0.8 and 90.8% with a score ≥0.95. 1.6% of simulations did not reach a score ≥0.8. Wrong diagnoses were called in only 1.3% of simulations with a score ≥0.8 and 0.5% with a score ≥0.95. Taken together, these results suggest that a conclusive diagnosis can be reached within 25-50 minutes of simulated sequencing for the vast majority of pediatric cases that can be classified using the Heidelberg v11b4 classifier, with a very low error rate.

The majority of misclassifications (141 out of 155 at timepoint 3) occurred in the simulated sequencing experiments from two samples (FIG. 28A). In both cases the misclassification occurred within the same family (PMC_38: Glioblastoma subtype Midline misclassified as subtype H3K27 mutant, PMC_64: Medulloblastoma subtype Group 4 classified as subtype Group 3).

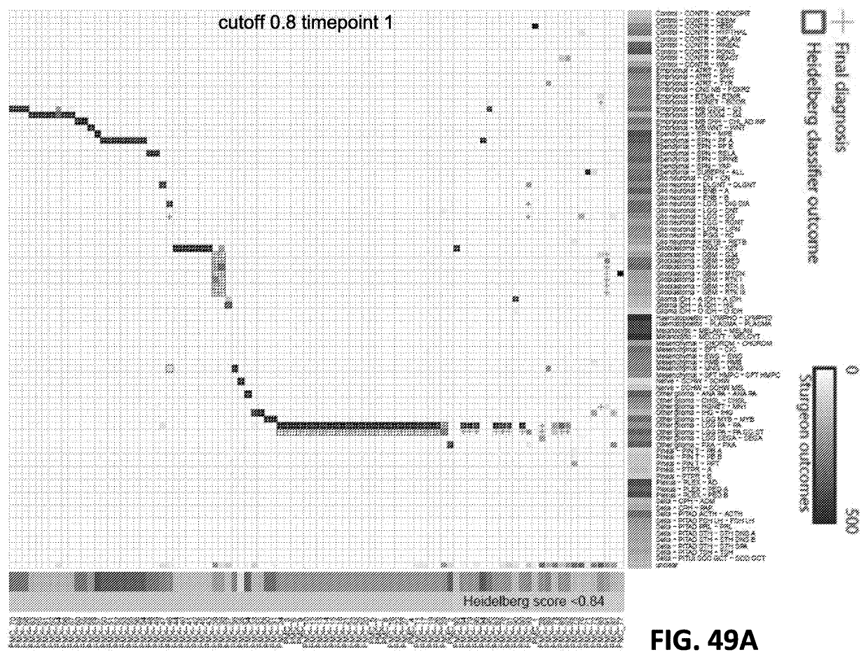

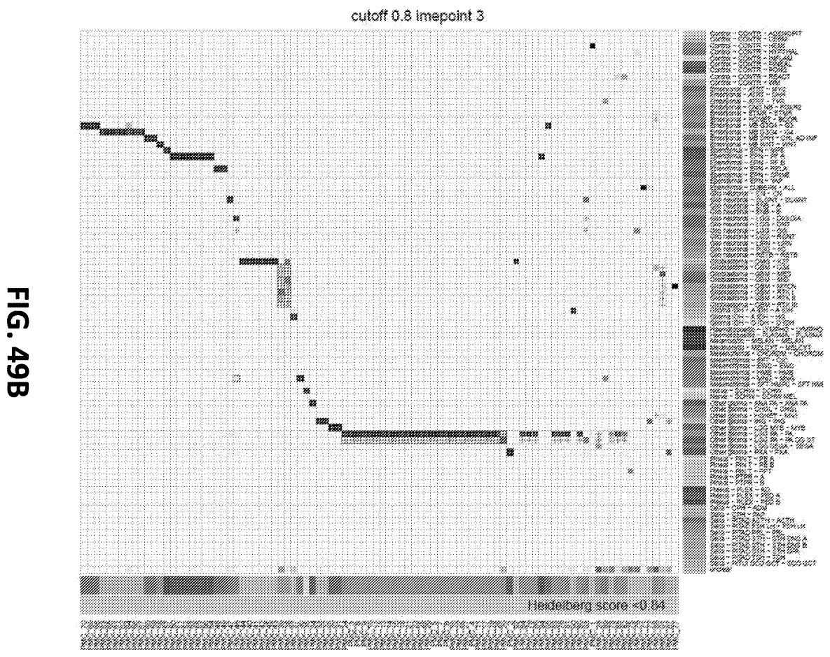

For the difficult to diagnose cases, Sturgeon was less performant in general. For most of these cases a definitive diagnosis was reached based on the combination of molecular and histological features. In 11 of the 26 cases Sturgeon reached a diagnosis in concordance with the definitive diagnosis (but often at later time points) in the majority of simulations. All of these 11 cases also reached a Heidelberg classifier score between 0.6 and 0.84 (FIG. 28B). In the remaining cases, both Sturgeon and the Heidelberg classifier performed poorly, most frequently resulting in an unclear diagnosis (low confidence scores or high confidence scores for control tissue classes). This can be attributed to different reasons: low tumor fraction based on histology (PMC_1, PMC_28, PMC_82 and PMC_76); classes not present in the 2018 classification scheme (PMC_71, PMC_73, PMC_77 and PMC_88); no definitive diagnosis (PMC_72 and PMC_75); or tumors in the context of a germline mutation (PMC_89, PMC_85, PMC_91 and PMC_77), which has been suggested to complicate methylation-based classification¹⁵ (see FIGS. 48A-49B for more details).

Together these simulation results indicate that Sturgeon can perform on par with the Heidelberg v11b4 classifier, even when applied to a very sparse simulated sequencing run. It also reiterates the limitation that Sturgeon (as any other machine learning-based classifier) is only able to perform well in samples that are sufficiently represented in the training data. Reassuringly, for classes that are not represented in the training data, confidence scores are usually low, resulting in an unclear outcome rather than a misdiagnosis.

### Sample Purity Affects Sensitivity

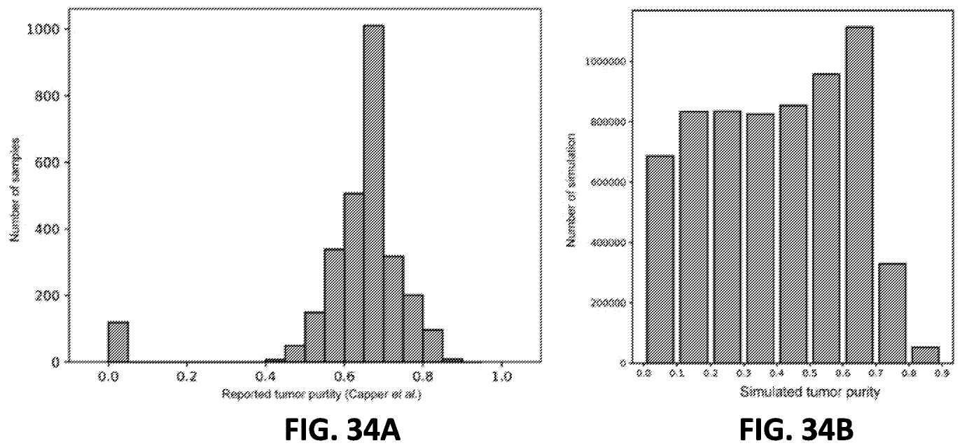

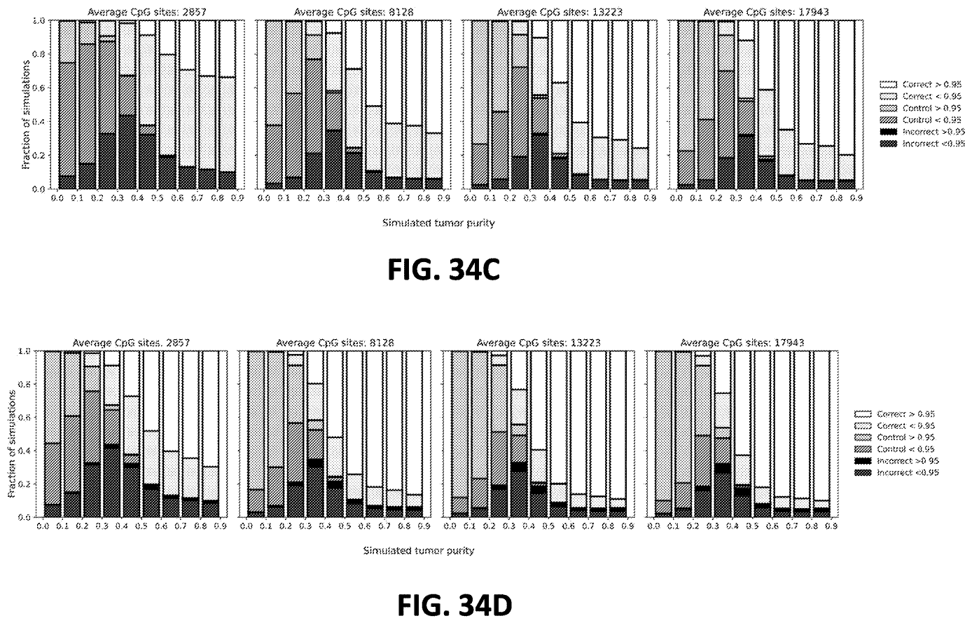

In an intraoperative setting, time constraints do not always allow for sample selection based on purity and samples may therefore contain a larger fraction of normal cells. In contrast, the training dataset consists of samples that are relatively pure, with the tumor cell content ranging from 40% to 85% (FIG. 34A). It was therefore aimed to further explore the behavior of Sturgeon on low tumor purity samples by in silico mixing simulated nanopore reads from one of the control tissues included in the Capper et al. dataset with simulated nanopore reads from a non-control sample (FIG. 34B and Methods). Simulations show that, as expected, a higher fraction of admixed control reads reduces performance, increasing the number of cases for which the classifier does not reach a confident classification. At lower (<50%) tumor fractions, the number of cases for which the control class is predicted increases (FIGS. 28C-D, FIG. 34C). Importantly, admixing control tissue reads does not lead to significant numbers of misclassifications, indicating that high scores are reliable, even when the tumor fraction is unknown. Deeper sequencing does not seem to resolve the difficulties in classifying low tumor fraction samples (FIG. 28D). To estimate how frequently this lower limit would not be reached in clinical practice metadata from 44 cases where intraoperative histology was performed was retroactively collected. In 6/44 pediatric cases the pathologist estimated the tumor fraction to be below 50%, 5/44 cases the pathologist estimated the tumor fraction to be around 50% and in 31/44 cases the tumor fraction was estimated to be above this threshold. In two cases the tumor cell fraction was not estimated. For adult glioma cases the mean tumor purity was estimated to be 69% in samples obtained from the FLAIR enhancing region²⁶, indicating that in these types of samples, material of adequate purity can be obtained more consistently. Based on these results, it was expected that in the intraoperative setting, especially in pediatric cases, some samples may not be classifiable due to low tumor purity. Low tumor purity is not expected to result in misdiagnosis in these samples when using the most stringent cutoff.

### Classification of Nanopore Sequenced Samples

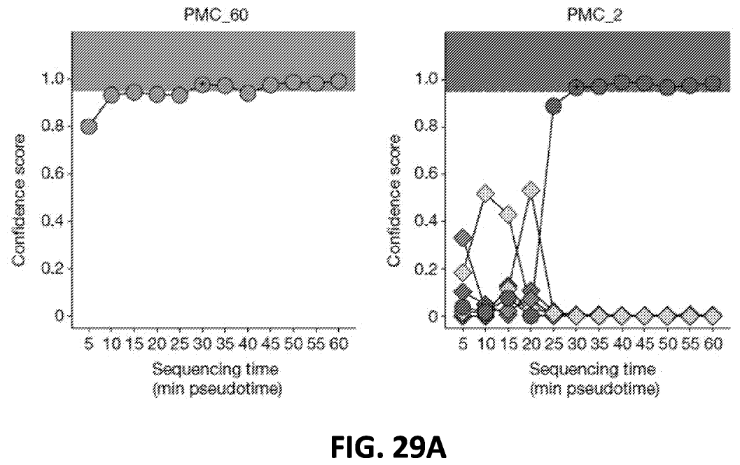

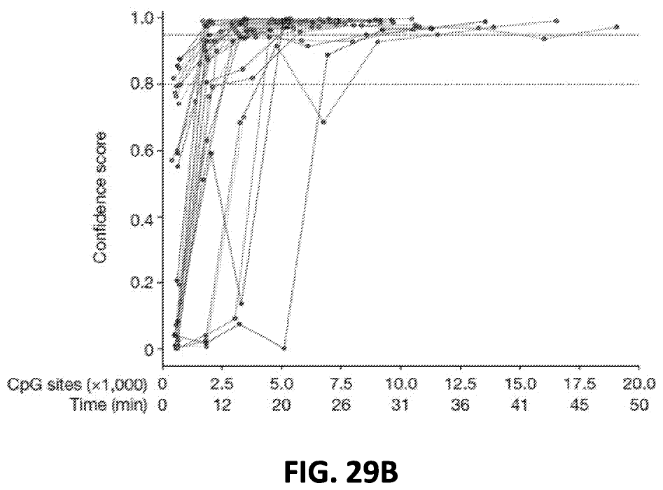

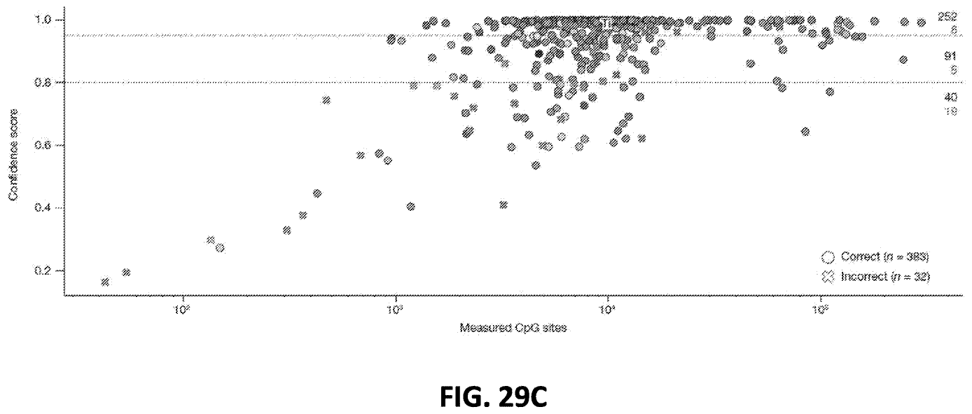

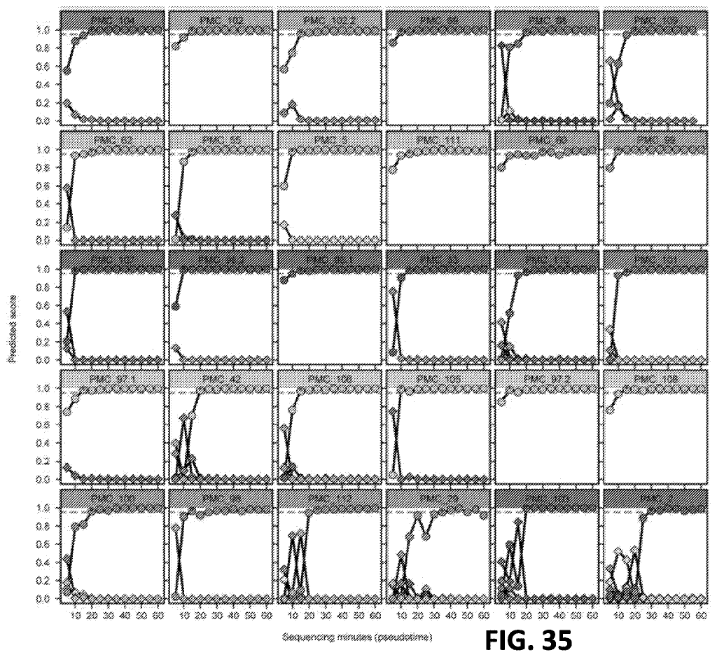

Next, the performance of Sturgeon on real nanopore sequencing data was assessed. 27 pediatric brain tumor DNA samples obtained from the PMC biobank were retrospectively sequenced and classified. Sturgeon was applied to increasing numbers of reads, mimicking an average MinION sequencing experiment in 5 minute intervals (Pseudotime) (Methods, FIG. 29A and FIG. 35).

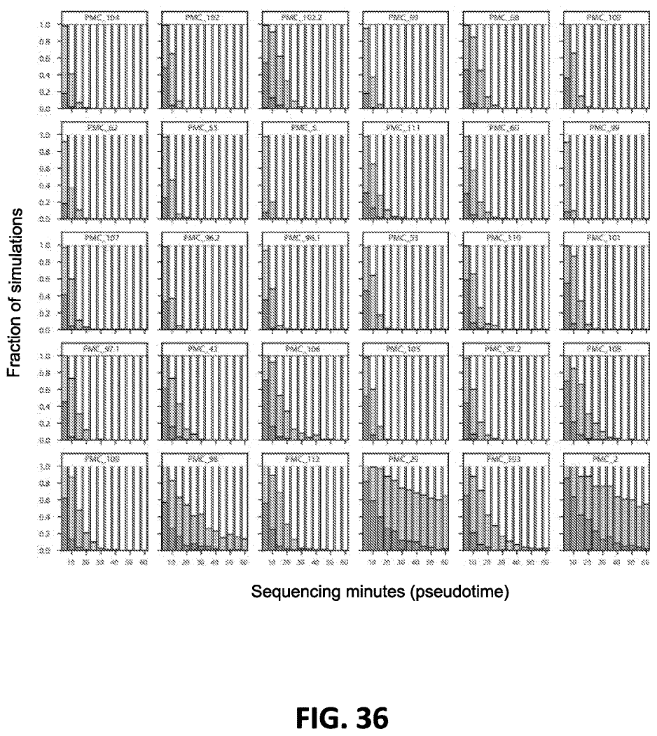

The classification results demonstrate that for 24 out of 27 samples Sturgeon assigned a score higher than 0.95 to the correct class after the equivalent of 25 minutes of sequencing; and on average this threshold was achieved between 15-20 minutes of sequencing (FIG. 29A). Samples PMC_2, PMC_60, PMC_29 and reached the 0.95 threshold at 30, 30, and 35 minutes of sequencing, respectively. For the majority of samples, ˜200,000 reads (˜3.9 Gigabases) were generated, where a typical intraoperative run (60 minutes of sequencing) would be expected to yield ˜60,000 reads (˜200 Mb) of throughput. This allowed us to evaluate the robustness of the results by randomly subsampling sequence reads, essentially simulating a different order in which the DNA molecules were sequenced. The results show that Sturgeon is very robust, reporting the correct class in 27,980 (score ≥0.95) out of the 36,000 predictions (77%); and only confidently reporting the incorrect class 14 times (0.03%). The outcomes are more confident and accurate if more sequence reads are available (FIG. 36). It also showcases how some samples (for example PMC_2 and PMC_29) are more difficult to classify than others.

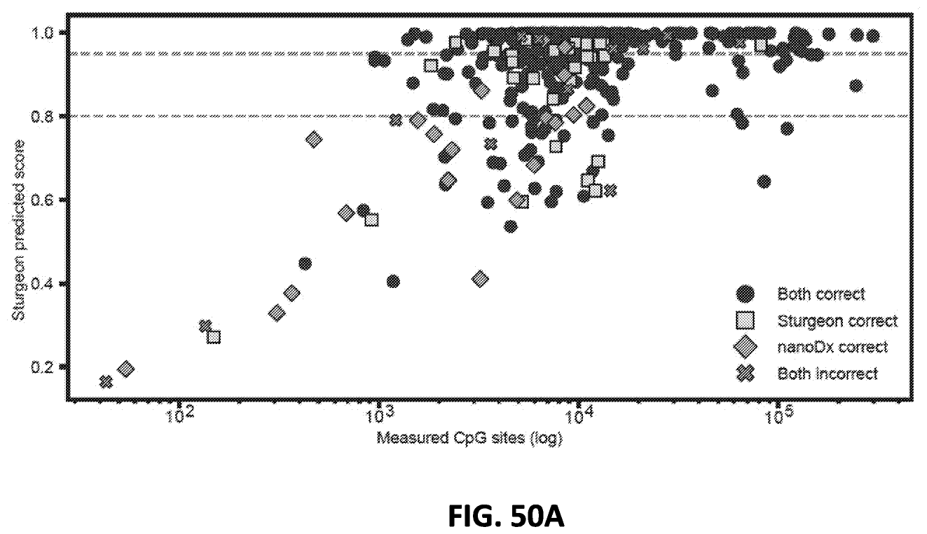

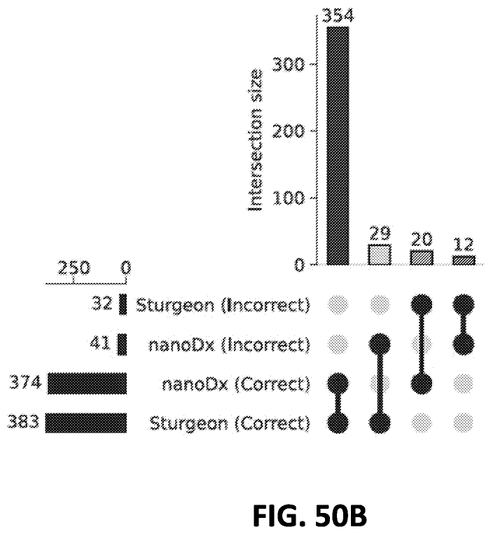

To further assess Sturgeon robustness and susceptibility to batch and operator biases, it was validated on a publicly available dataset²² (GSE209865) consisting of nanopore sequencing data for 415 CNS tumor sequencing runs, corresponding to 382 unique samples (FIG. 29C, FIGS. 50A-50B) from a variety of cases, including adult patients. The provided dataset is already processed, where methylation calling is performed using Nanopolish (rather than Megalodon/Guppy, which was used in the default workflow), and probe methylation status is already mapped. It was found that, despite these differences in sample workflow, Sturgeon still performed as expected, even slightly outperforming nanoDx, the patient-specific random forest classifier used in the original publication, as it is able to correctly predict 9 additional samples (FIG. 50B). Sturgeon correctly classified 383 (92.2%) samples, 343 (82.6%) at a confidence threshold ≥0.8 and 252 (60.7%) samples with a confidence ≥0.95. From the 415 samples, 32 (7.7%) were incorrectly classified, 8 (1.9%) of which reached a confidence score ≥0.95 FIG. 29C, FIG. 50A). It was noted that, for 5 of these 8 confidently incorrectly classified samples: 4 corresponded to a single sample which is also incorrectly classified by nanoDx; and 1 is incorrectly classified as an STH producing Pituitary Adenoma instead of TSH producing Pituitary Adenoma. Overall, nanoDx is able to perform better in scenarios with extremely low coverage of CpG sites, due to its patient-tailored model. However, Sturgeon performs better and is more confident with a CpG site coverage compatible with intraoperative sequencing (FIGS. 50A-50B).

### Copy Number Variations

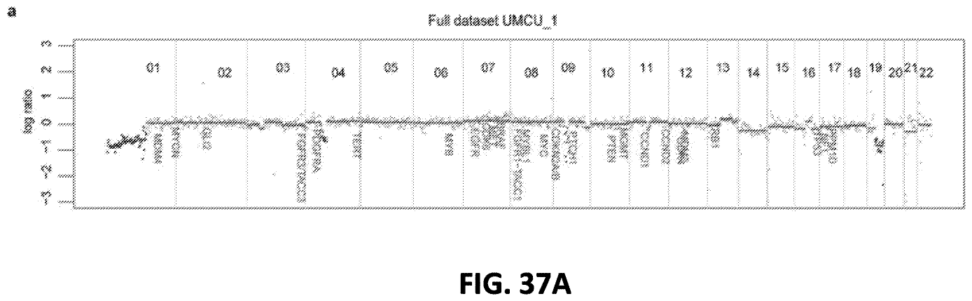

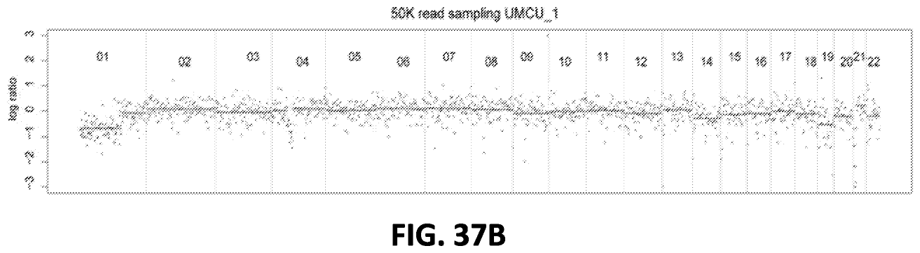

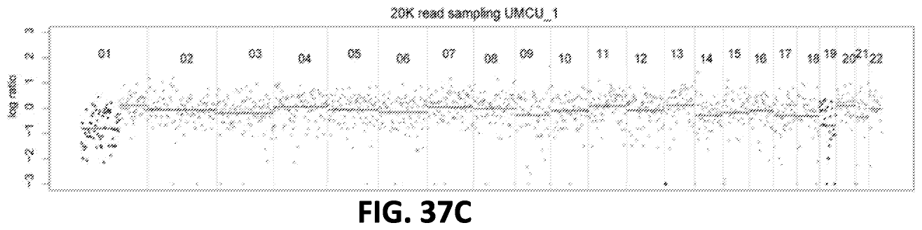

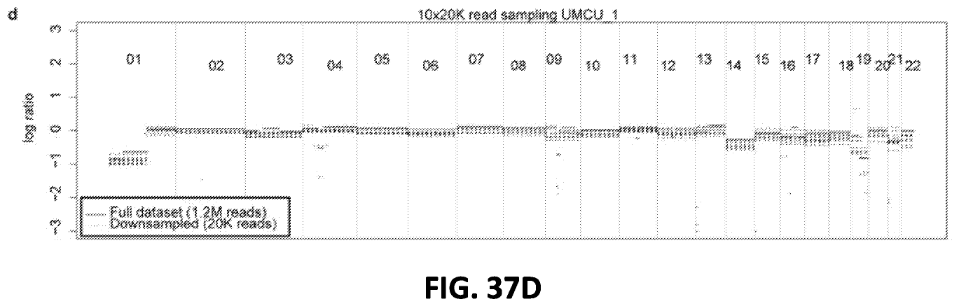

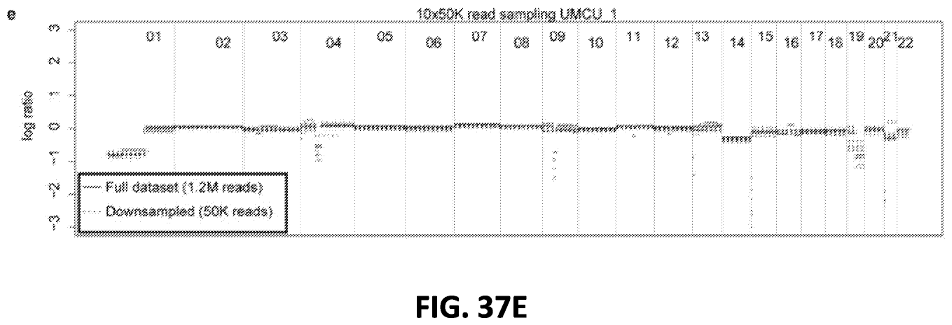

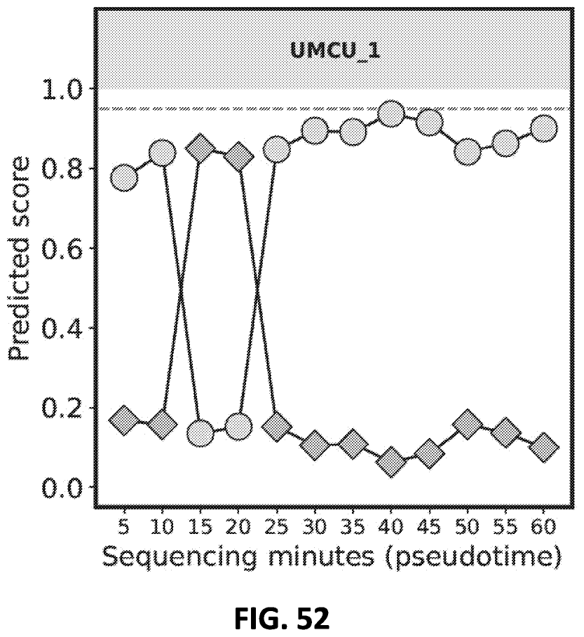

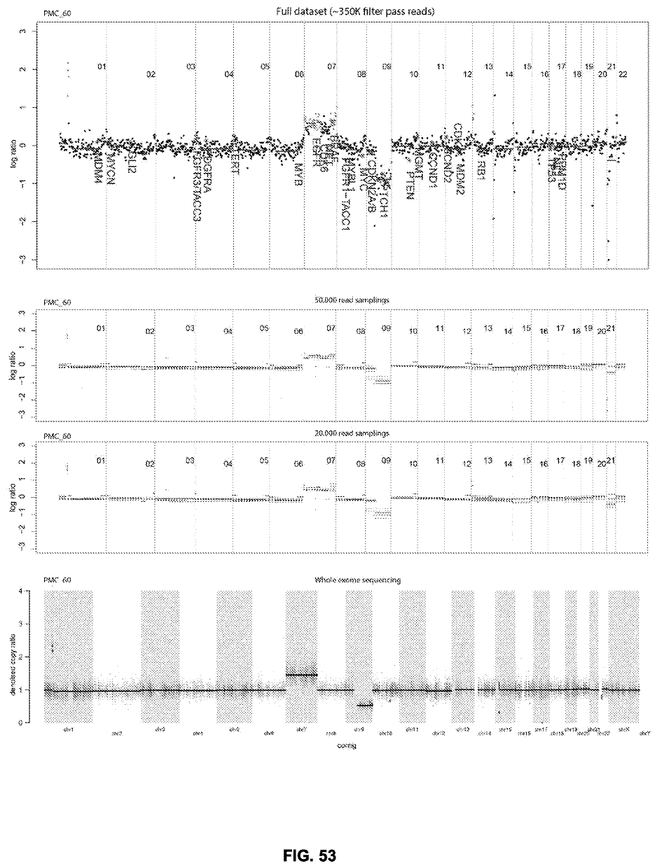

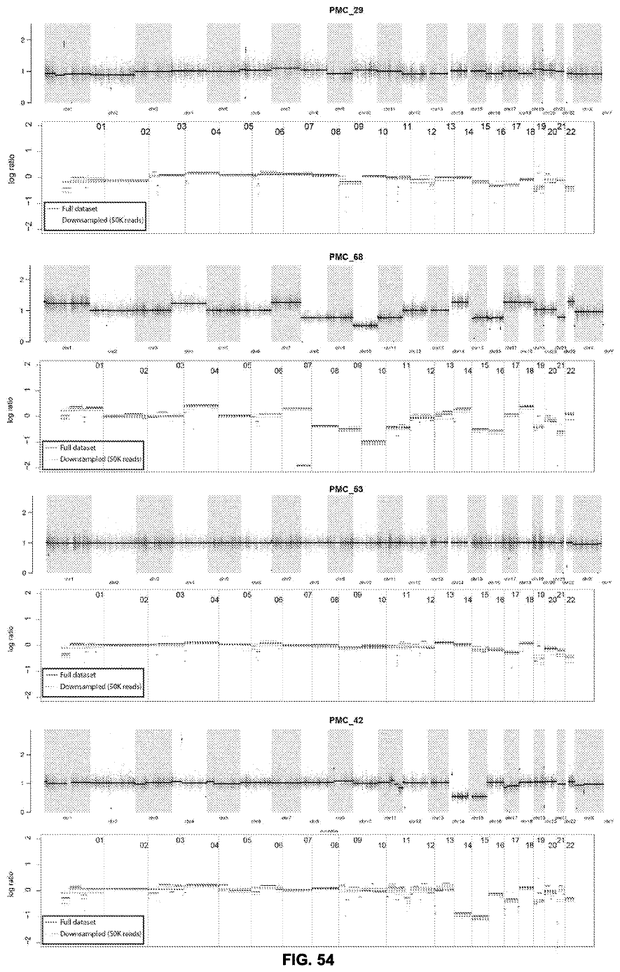

In addition to methylation profiling, copy number variations (CNVs) play an important role in tumor classification, prognosis and downstream treatment. It was explored whether CNVs could be detected using shallow nanopore sequencing, to further support the classifications provided by Sturgeon. For example differentiating between IDH-mutant Oligodendroglioma and Astrocytoma can be challenging (FIG. 52), and in such cases a 1p/19q codeletion is clear evidence of the former. To this end, the approach published by Euskirchen et al.²⁷ was adapted. Using a downsampling approach there was an ability to detect large scale CNVs such as the 1p deletion from as little as 20,000-50,000 sequence reads (Methods, FIGS. 37A-37E), although smaller CNV's such as the 19q deletion are less reliably detected. The clinical relevance of different CNVs is context dependent, and therefore the example of the Heidelberg classifier was followed: CNV plots were provided in the workflow to the pathologist in parallel to the methylation classifier result for further interpretation. More examples of nanopore sequencing derived CNV profiles are shown in FIGS. 53-54.

### Intraoperative Sequencing

To demonstrate the clinical feasibility of Sturgeon in an intraoperative sequencing context, the protocol was performed during 25 surgeries at two different Dutch hospitals. This comprises 20 pediatric samples, performed at the PMC and 5 adult samples, performed at the Amsterdam University Medical Centers (AUMC). Samples obtained for histological assessment during surgery were split, and one part was used for intraoperative sequencing while the other part was used for histological assessment. In order to rapidly obtain a high concentration of input DNA, the DNA extraction protocol was optimized to extract DNA from fresh tissue samples in 17-20 minutes (Methods).

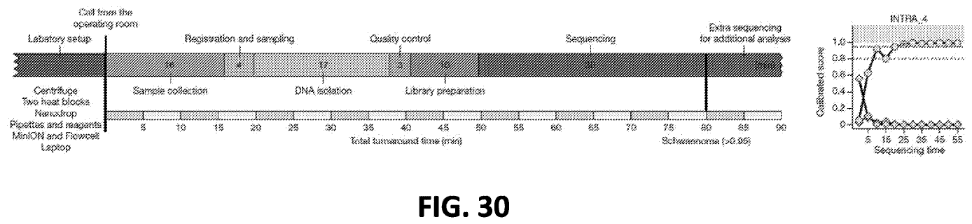

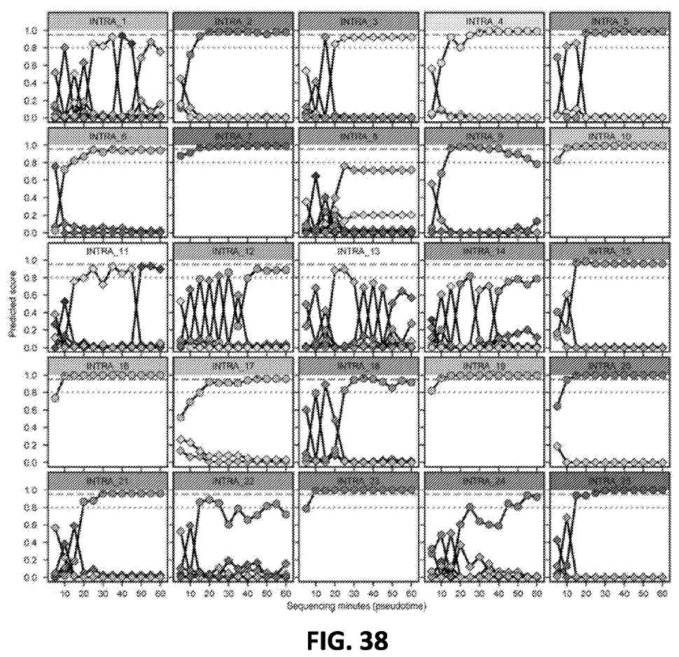

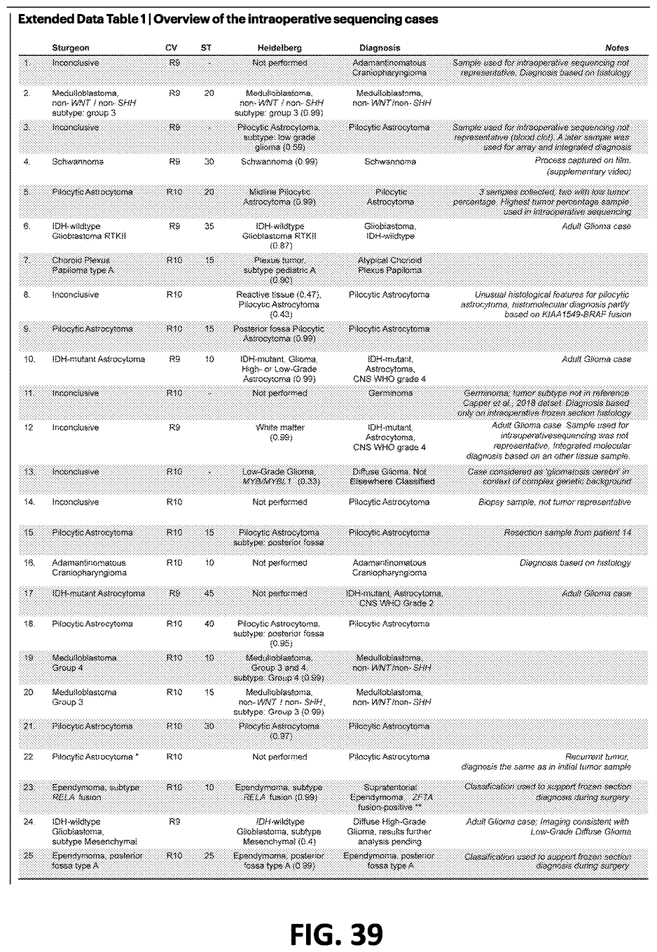

FIG. 39 lists the results and context of the 25 intraoperative sequencing experiments (FIG. 38 shows the scores as they developed over time). FIG. 30 shows the timeline for this particular case.

For the five CNS tumor samples from adult patients, glioma cases were specifically focused on as surgical strategy may affect the outcome differently in IDH-wildtype versus IDH-mutant High-grade Gliomas^{28,29}. In cases with a High-grade Glioma, showing enhancement on T1 contrast-weighted MRI, intraoperative fluorescence (5-aminolevulinic acid (5-ALA) is used to mark tumor cells during resection³⁰, allowing consistent sampling of high tumor content samples and resulting in successful classification in 4 out of 5 cases where tumor was sampled.

In summary, Sturgeon was able to correctly classify 72% of tumors (18 out of 25) at the subclass level with at most 45 minutes of sequencing. In the other cases the classification was unclear, which can be attributed to tumor classes not present in the reference dataset (INTRA_11), low tumor purity (INTRA_1, INTRA_3, INTRA_14, INTRA_15) or exotic cases (INTRA_8, INTRA_13). Several cases where a rapid molecular classification would have been of significant added value were encountered. For example, in cases INTRA_23 and INTRA_25 the tumor class was not known prior to surgery. Intraoperative frozen section diagnosis suggested an Ependymoma in both cases, which indicates that a radical resection is the best course of action. Sturgeon soon after that achieved a high confidence for Ependymoma subtype RELA fusion and Ependymoma subtype A respectively. Both classifications were later confirmed using a methylation array and the Heidelberg V11b4 classifier. Obtaining the molecular class intraoperatively in these cases provided an independent classification that corroborated the intraoperative frozen section diagnosis, reducing the risk of a misdiagnosis and providing additional certainty for the neurosurgeon to follow the best surgical plan for these patients.

### Location Specific Models

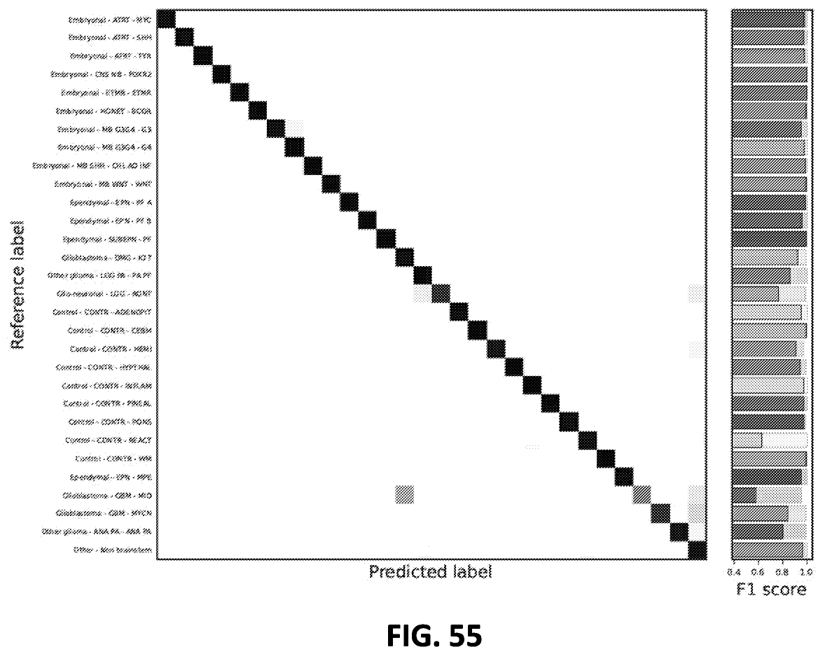

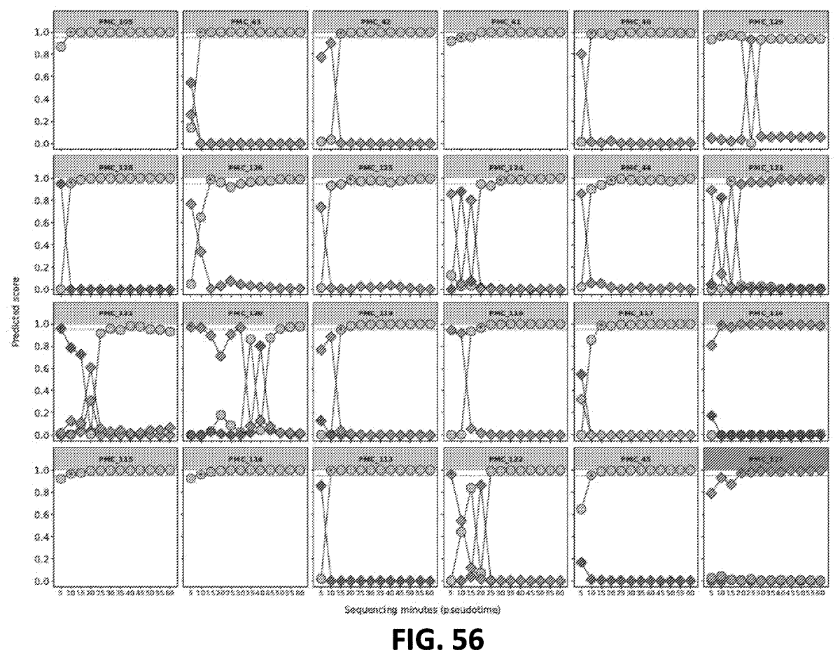

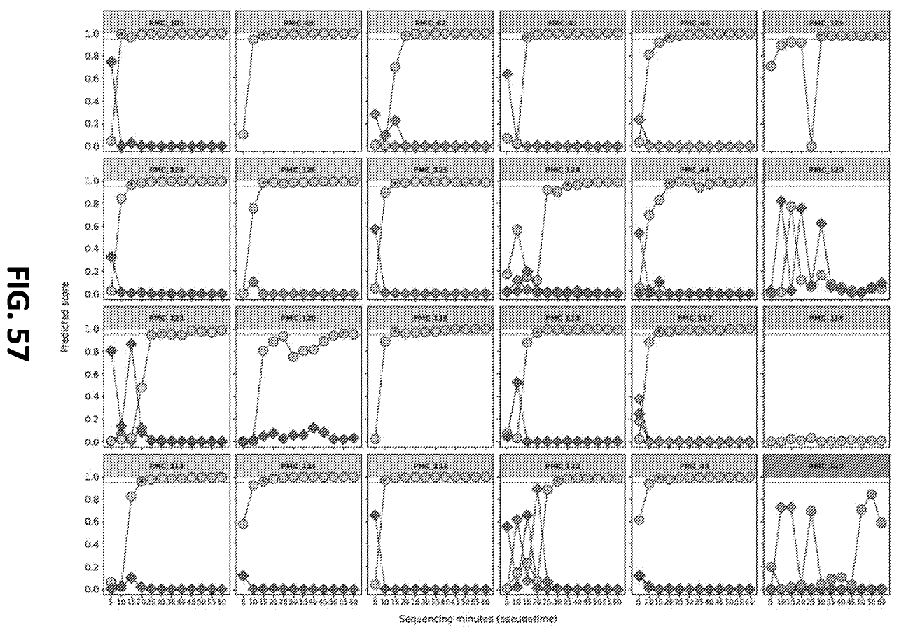

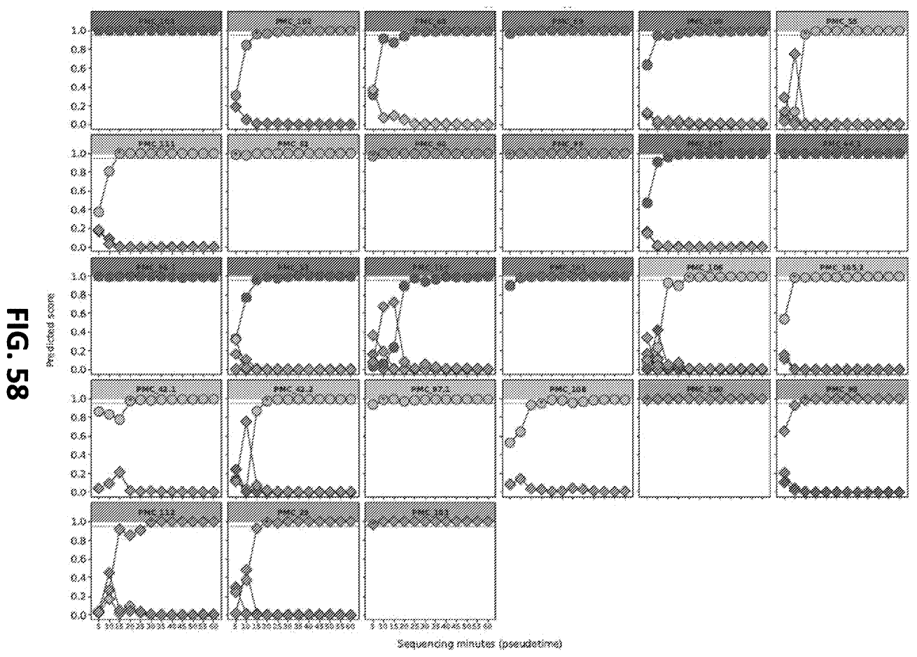

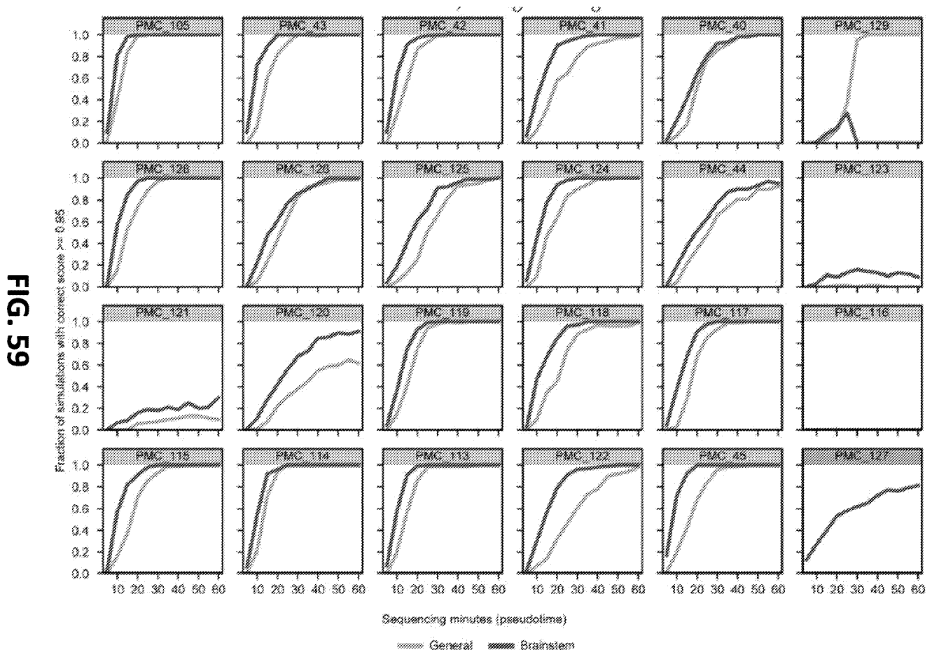

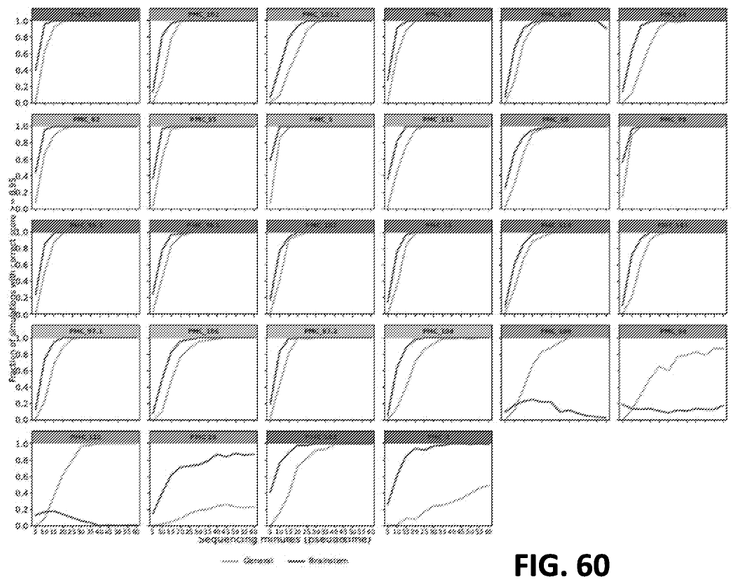

The Capper et al. dataset¹⁴ encompasses 81 tumor classes. However, many class distinctions are only relevant within a particular topological context, and could therefore already be ruled out prior to surgery. For instance, for a surgery of the spinal mass, a classifier does not need to be able to detect Pituitary Adenomas. Compared to other regions in the brain, the number of relevant classes in the brainstem is relatively low (N=21). It was hypothesized that, by merging the irrelevant classes from the training dataset into a single class, the model can focus on the truly relevant classes and improve its performance. To test this hypothesis, a Sturgeon classifier was developed specifically for brainstem tumors. In summary this adjusted approach can slightly improve the required sequencing depth. The design, validation and results are shown in FIGS. 55-60).

### Adaptive Sampling

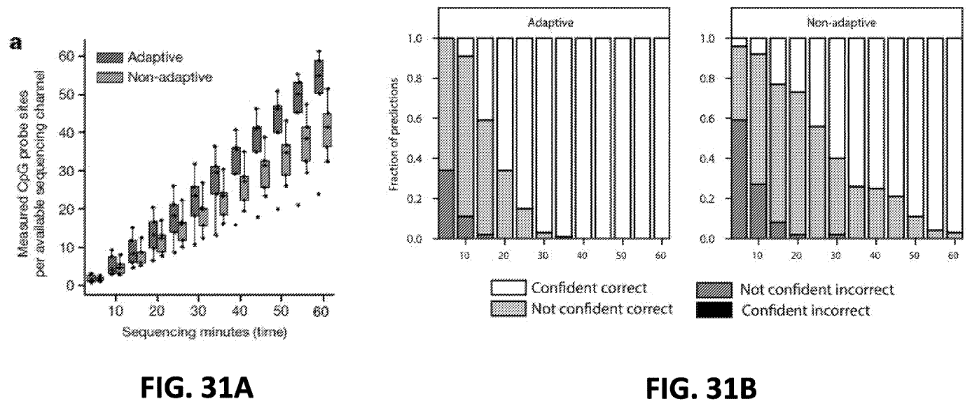

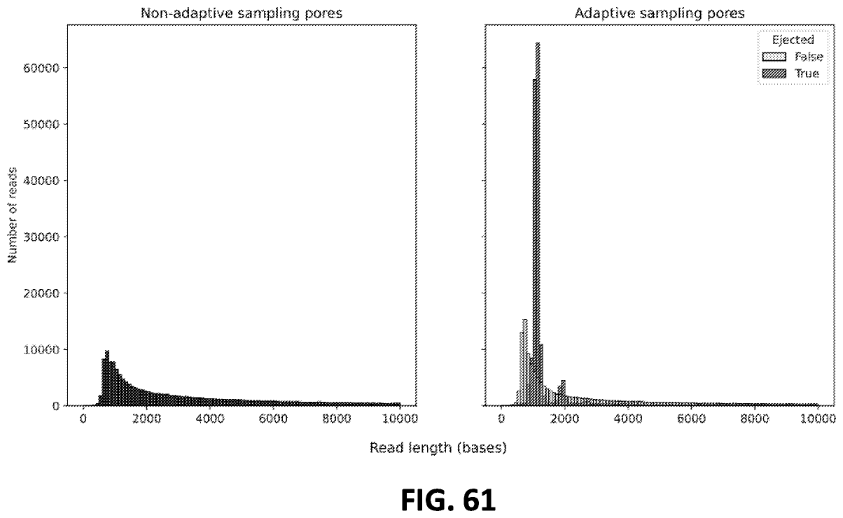

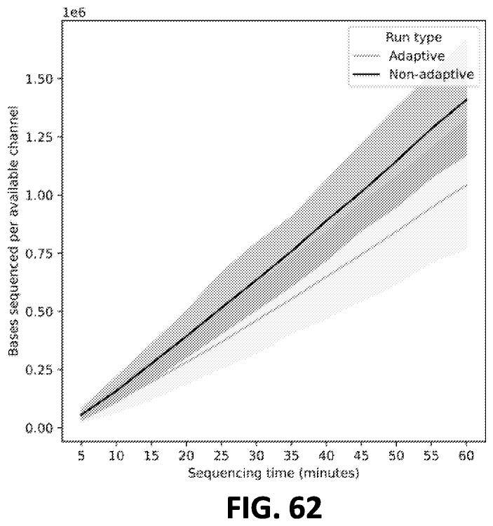

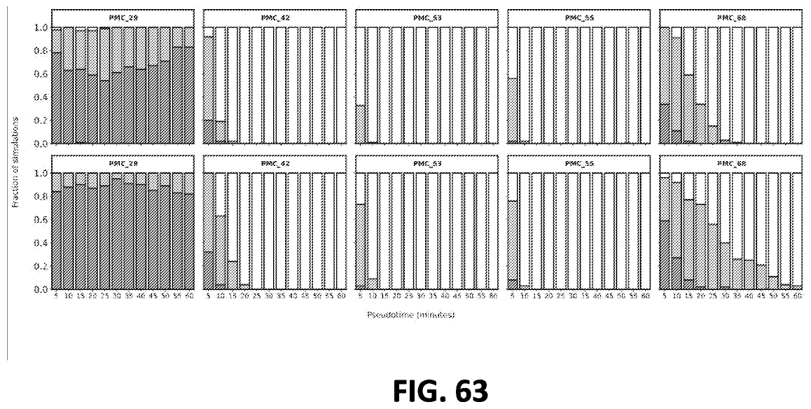

With the most recent chemistry, nanopore sequencers are able to reverse the current in specific channels, thereby ejecting reads as they are being sequenced. This has enabled ‘adaptive sampling’, for example, where a read is mapped when the first ˜400 bases are sequenced, and subsequently rejected if it falls outside any of the targeted regions³¹. This strategy can improve the TAT by rejecting reads that are unlikely to overlap informative CpG sites. An adaptive sampling strategy (Methods) was designed and tested this in five samples. Overall, the number of informative CpG sites sequenced per minute is 15-30% higher with adaptive sampling, and Sturgeon is more confident in classifying the same sample (FIGS. 31A-31B, and FIGS. 61-63). However, it is noted that adaptive sampling can be technically challenging to set up on custom hardware, and comes with an increased hardware requirement. For simplicity, it was not used in an intraoperative setting.

The use of adaptive sampling in combination with the methods disclosed herein provides various advantages in terms of effectively speeding up the time taken to sequence. Adaptive sampling has been disclosed previously, for example in US Pat. App. Pub. No.: 2017-233804, herein incorporated by reference in its entirety, whereby a polynucleotide strand is compared to a reference during measurement of the strand in a nanopore to provide a measure of similarity and responsive to the measure of similarity, the sensor element may be selectively operated to eject the polynucleotide and thereby make the nanopore available to receive a further polynucleotide. This has the overall effect of speeding up sequencing by rejecting unwanted polynucleotides. The use of adaptive sampling in combination with the methods disclosed herein provides various advantages in terms of effectively reducing the time taken to perform the sequencing thereby reducing the overall time taken to classify a tumour. In an embodiment, adaptive sampling may be performed during nanopore sequencing.

### Discussion

Here it was demonstrated the practical feasibility of intraoperative methylation-aware nanopore sequencing for pediatric CNS tumor classification which can be used to improve surgical decision making. Classification during surgery is challenging because due to the short sequencing time only very sparse data is available and it is a priori unknown which CpG sites will be covered. Furthermore nanopore-sequenced reference samples are not widely available.

To address these challenges, Sturgeon was developed, a deep learning approach that is trained on simulated nanopore sequencing data, generated from readily available methylation array data, and can accurately classify tumor types based on intraoperatively generated sequence data. Sturgeon uniquely moves the computationally intensive model training, validation and calibration phase outside the surgical time window, providing well-tested highly accurate one-size-fits-all classification models. Contrary to previous approaches^{4,22}, Sturgeon models are not patient specific and can be used universally without retraining, mitigating the need to have access to privacy sensitive training data at the site of deployment. As a result, only limited computational resources are required during surgery. For example, the Sturgeon classifiers shown here can classify a Megalodon output file containing data from 32,610 reads in 17 seconds on an AMD Ryzen 7 6800H CPU. As the model inference step practically poses no constraint on the time it takes to classify a sample, it is possible to run multiple Sturgeon classifiers in parallel. Furthermore, it can be shown that the models perform robustly across different sequencing flowcell types (MinION and PromethION, using R9 and R10 chemistry), laboratories (Utrecht, Amsterdam and Oslo) and methylation calling methods (Megalodon/Rerio, Guppy/Remora and Nanopolish). However, as any other methylation-based classifier to date, performance is limited by tumor purity in the analyzed sample, and cannot account for intratumor heterogeneity.

We envision training of improved versions of Sturgeon as more data becomes available. The class definition used in the Capper et al. data, used for training Sturgeon, has since been updated several times, with the addition of many new classes³². When these data, or data from in-house cohorts are available, re-training and/or fine-tuning the Sturgeon model is straightforward. Leveraging data from many different institutes across different countries for training machine learning algorithms is complicated due to data sharing restrictions as a result of privacy legislation that follows from the patient's consent. Sturgeon is ideally suited to address this as it can readily be employed in a federated learning setting³³. Moreover, due to the simulation approach employed by Sturgeon, it may be envisioned that different types of training data, such as those obtained using other microarray platforms, nanopore or bisulfite sequencing can all be naturally accommodated.

Ultra-fast methylation sequencing holds great potential for several other fields of application. For routine (post-operative) diagnostics, the TATs can be significantly shortened, reducing patient distress and anxiety and allowing tailored treatments to start as soon as possible. Furthermore the low investment cost enables application in peripheral centers and centers with limited financial means. A longer-term future application may be to support administration of implantable therapeutics, which have the potential to bypass the blood brain barrier. Current applications are associated with a high complication rate³⁴ and are so far limited to recurrent tumors³⁵, where the specific tumor type is known.

A potential limitation of the Sturgeon approach is the required sample mass. So far, surgeons have been instructed to obtain a sample measuring roughly 5×5×5 mm, as this yields a high concentration of DNA suitable for library prep. It has, however, successfully extracted sufficient DNA from smaller samples, including a biopsy. It may be noted that, in particular when using the R10 workflow, as little as 200 ng genomic DNA is sufficient. However, for specific applications, such as needle biopsies, more sophisticated (but slower) extraction protocols may be required to obtain sufficient DNA.

In conclusion, the results demonstrate that TATs of 1.5 hours are feasible for the majority of samples. This is fully compatible with the timeline to guide the (neuro)surgeon on how to proceed with the surgical procedure. A clinical application of Sturgeon may be envisioned such that it is deployed parallel to histological assessment by a trained pathologist who then integrates the histological and molecular results into an improved intraoperative diagnosis. Using Sturgeon in this way could also reduce the requirement for a confidence score of ≥0.95 since the pathologist will always weigh the predicted tumor class in the context of the observed tumor histology, patient history and tumor location. Sturgeon can especially play an important role in guiding decision making in challenging cases where the histological picture is ambiguous.

### Example 6: Methods

### Data Simulation

Short nanopore sequencing runs yield sparse and random coverage of the genome. To enable model training, simulated sparse nanopore runs based on microarray data were generated. To this end, N simulated reads are randomly sampled from the read length distribution (D) and assigned a start mapping position in the genome. N and D are defined based on an average nanopore whole genome sequencing run using a MinION flowcell (FIGS. 27A-C). Forward or reverse direction is chosen at random (50% chance each). Reads are clipped at the start/end of the chromosome. Given this set of reads, the covered CpG sites are determined and their binarized methylation status is obtained from the microarray sample. Raw EPIC profiles were binarized with a cut-off of beta ≥0.6 using scripts kindly provided by the authors of nanoDx²². To include measurement noise due to sample heterogeneity, and methylation calling error rate, 10% of the covered CpG sites are randomly flipped. To reduce overtraining on specific sparsity levels, runs of different sparsity levels in a balanced manner (see below) were simulated. To ensure reproducibility and avoid simulation leakage between samples of the different cross-validation folds, simulations can be completely deterministic (with the exception of noise) given a random seed and the simulation time.

### Cross-Validation

To assess model performance the Capper et al. dataset¹⁴ is split in four equally sized class-stratified folds. Two folds are used for submodel training, one for validation to assess the best model state during training and to perform score calibration. The final fold is used for testing to assess the submodel performance. Folds are rotated so that a total of four submodels are obtained. Simulations are tightly controlled through the seeds of the pseudo-random number generator—that is, training, validation and test seeds are mutually exclusive—to avoid cross-validation leakage. Seed values between 0-499 were used for the test fold, between 500-999 for the validation fold and between 1,000-1,001,000 for the training fold.

### Neural Network Architecture

Sturgeon (named thus to fit in the traditional fish-based nomenclature for nanopore software and because it sounds like ‘surgeon’) is a neural network containing three fully connected layers. The first two layers have 256 and 128 dimensions respectively, and are followed by a sigmoid activation (FIG. 23B). The first layer has an input size of 428,643, corresponding to the number of probes on the arrays used in training. The last linear layer has a dimensionality equal to the number of classes to be predicted (91 dimensions for the general classifier, and 30 dimensions for the brainstem-specific classifier). The outputs of the neural network are calibrated by a learned scalar value (see ‘Score calibration’), and then transformed to probabilities via the softmax function. Dropout rate between layers was set to 0.5. As classification loss cross-entropy with uniform weights was chosen.

### Submodel Training

The neural network was trained as a supervised multi-class classification problem. Longer have therefore easier to classify; shorter simulations are more difficult. Therefore, a curriculum learning approach was used, where a mix of easy and difficult simulations are used to start the training the neural network, and later on move to train on only more challenging ones. Therefore, the neural network is first pretrained on the Capper et al. samples¹⁴ (91 classes) using simulations that range between 0.6% and 14% sparsity (this range contains both easy and difficult to classify simulations). The neural network is fine-tuned for the final classifier by training using simulations that range between 0.6% and 6.3% sparsity (this range contains more difficult to classify simulations). For the brainstem classifier, the last layer is substituted by an untrained layer with the correct dimensionality (30 classes). The neural network is pretrained for a total of 3,000 epochs with a batch size of 256. For this purpose the AdamW³⁶ optimizer is used with a starting learning rate of 10⁻⁵ that increases linearly for the first 1,000 training batches until 10⁻³; afterwards, it is decreased using a cosine function until it reached 10⁻⁴ on the 1,000th epoch; it is kept training at a constant learning rate of 10⁻⁴ for 2,000 epochs. Other parameters of the optimizer are: β₁=0.9, β₂=0.999, ε=10-8 and λ=0.0005. During training, a dropout rate of 0.5 between all layers is applied. One epoch is defined as the number of reference samples in the most abundant class multiplied by the number of output classes. For every 2,000 training batches the current weights of the model are saved; and the model is evaluated on 50 validation batches (12,800 samples) by calculating their average loss and sensitivity.

### Validation

Batches are sampled in the same manner as the training batches, with the exception that simulation seeds are independent. The neural network is fine-tuned using the exact same parameters as described for the pretraining, with the exception that it is fine-tuned for 3,000 epochs with a constant learning rate of 10⁻⁴. During inference, samples were classified using the four trained submodels and use as final classification the scores from the model with the highest confidence. For the general classifier the scores of two highly similar pilocytic astrocytoma classes (posterior fossa pilocytic astrocytoma and midline pilocytic astrocytoma into a single class low grade glioma pilocytic astrocytoma) and two highly similar medulloblastomas (SHH medulloblastoma child/adult and SHH medulloblastoma infant into SHH medulloblastoma) are summed up.

### Adaptive Sample Balancing

Because of class imbalance in the training dataset, all classes are upsampled such that they are equally represented by simulating additional samples for classes smaller than the largest class. Similarly, the sequencing sparsity levels are balanced such that the training data for each class consists of samples that have a uniform distribution of simulated sequencing times. At the end of each epoch, the class balance is recalculated by increasing the upsampling of classes and/or simulation times for which the model performs worse. Conversely, classes and/or simulation times for which the model performs well are upsampled relatively less. The number of samples for each class (c) and sparsity level (t) for epoch i+1 is provided by:

$${NumSamples}_{\text{?}} ( i + 1 ) = TotalNumSamplesEpoch \times \frac{{Error}_{\text{?}} ( i ) + 0.3}{{\sum }_{\text{?}} {\sum }_{\text{?}} {Error}_{\text{?}} ( i ) + 0.3}$$

$$\text{?} \text{indicates text missing or illegible when filed}$$

A correction constant (0.3) was added to avoid completely removing classes or timepoints from the epoch. The total number of samples per epoch is kept constant, based on the first epoch.

### Score Calibration

To calibrate the classifier scores, enabling interpretation of these scores as confidence scores, temperature scaling²⁴ was used. To this end, each validation fold sample is used to create 500 simulated sparse samples (using all validation fold seeds) for all sparsity levels (between 0.6% and 14% sparsity). Given the whole reference dataset (2,801 samples), this results in 16,806,000 total simulations. Based on these simulated samples, scalar temperature parameter can be optimized, used for calibration, by minimizing the class-weighted cross-entropy between the temperature divided non-scaled logits and the correct class label. For this purpose the L-BFGS algorithm implemented in PyTorch was used with learning rate 0.01 and a maximum of 500 iterations. The calibration of the model was evaluated using ECE, a statistical measure that summarizes the difference between classifier accuracy (acc) and confidence (con). The ECE is defined as the bin-weighted average of the absolute difference between accuracy and confidence on equally sized bins B (here 10 bins were used).

$$ECE \text{?} {\sum }_{m \text{?} 1}^{\text{?}} \frac{\vert {B}_{m} \vert}{n} \vert acc ( {B}_{m} ) - conf ( {B}_{m} ) \vert$$

$$\text{?} \text{indicates text missing or illegible when filed}$$

### Model Evaluation

The final performance of the model on the left-out test fold samples was assessed. For this purpose, each sample was used to simulate 500 sparse nanopore runs across all 12 sparsity levels. In this way, each sample contributes 6,000 simulated samples to the test set. Top1 and top3 F1 scores were reported for each class individually across all sparsity levels, as well as average metrics across classes. F1 score, as an evaluation metric, is chosen since it considers both types of errors (false positives (FP) and false negatives (FN)), but it does only include true positives (TP), and not true negatives (since these would inflate the metric massively due to the multi-class setting). The TPR for each class was also reported.

$$F 1 score = \frac{2 \times TP}{2 \times TP + FP + FN}$$

$$TPR = \frac{TP}{TP + FN}$$

### Pseudotime

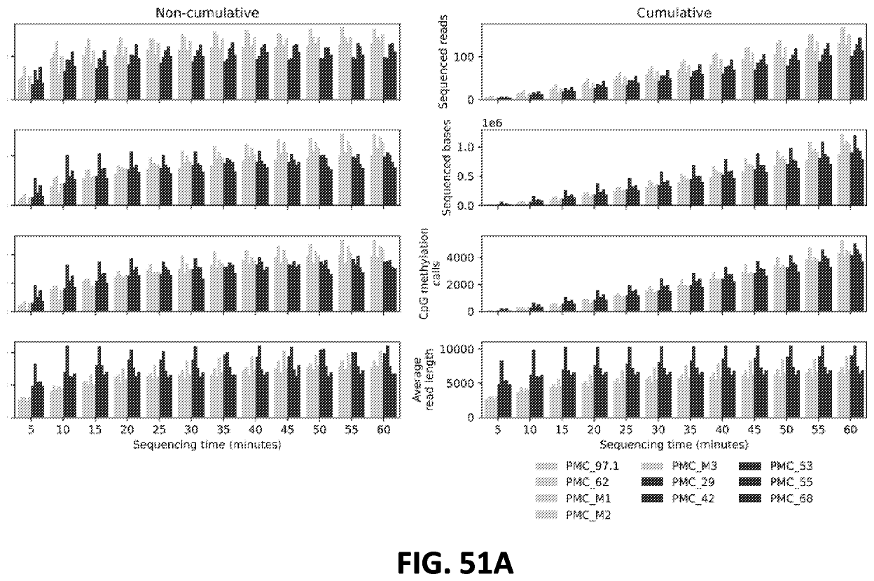

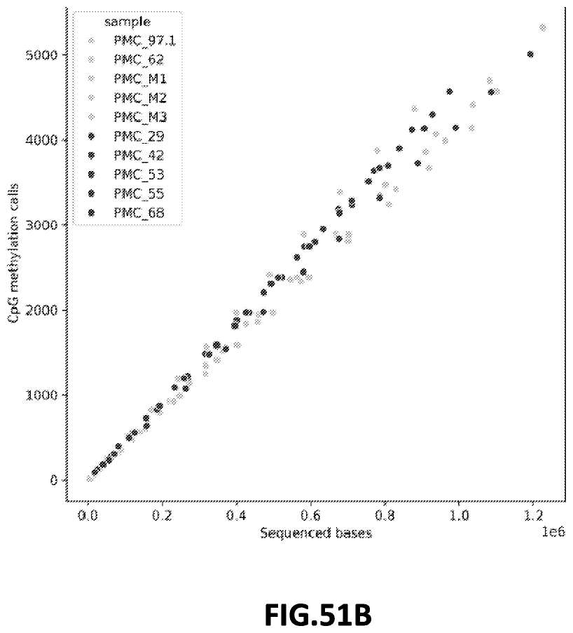

To reduce costs, some samples used for validation were sequenced on washed flowcells, or multiple samples were multiplexed on a single MinION or PromethION flowcell. Re-used flowcell and multiplexed sequencing times are not directly comparable to sequencing runs of a single sample on a new flowcell. Similarly, samples sequenced on PromethION flowcells are not directly comparable to MinION flowcells due to their larger throughput. In order to make these runs comparable to a real intraoperative scenario (one sample sequenced on a new MinION flowcell), the number of CpG calls was used as a proxy for sequencing time. For this purpose, it was first estimated the expected sequencing throughput in terms of the median number of CpG sites covered per 5 min time interval from a collection of 6 representative MinION runs that used fresh flowcells (FIG. 51). The number of CpG sites (non-cumulative) covered in the first 12 intervals of 5 min are: 51,924; 104,073; 124,078; 149,111; 173,504; 194,399; 207,456; 217,193; 232,101; 241,278; 247,600; and 258,197. Thus, for a multiplexed (MinION or PromethION) or washed flowcell sequencing run, a fresh flowcell equivalent may be assumed: 5 min of sequencing is achieved when 51,924 CpG sites are covered. By using throughput, instead of number of reads, this allows us to properly simulate the ramp up in sequencing throughput that happens during the first minutes of sequencing.

### Robustness to Random Sampling

To further analyze the robustness of Sturgeon, additional realistic nanopore sequencing data was created by randomizing the order of the sequenced reads. The sequenced read order of each sample was randomized 100 times and evaluate at which pseudotime the desired threshold would have been reached and whether the classification was correct or not.

### Robustness to Sample Purity

To analyze the robustness of Sturgeon to impure samples, nanopore runs were simulated with a mix of reads from tumor and control reference samples at different impurity levels. To achieve this, nanopore runs that contain between 5% and 95% control tissue reads in 5% increments were simulated. For each sample 100 independent nanopore runs were simulated, containing reads counts equivalent to between 10 to 40 min of sequencing. This produced a total of ˜20 million simulated sequencing runs. It was noted that the tumor reference samples are not 100% pure, and contain some levels of non-tumor tissue. The in silico purity was therefore reported by multiplying the original tumor fraction with the fraction of reads that are simulated from the tumor sample. The Sturgeon performance was evaluated by cross-validation, that is, each Sturgeon submodel was evaluated on simulations from samples and simulation seeds in the test fold.

### Paediatric Methylation Profile Validation

At the PMC centre for paediatric oncology, EPIC arrays are routinely performed on paediatric CNS cancer samples. 94 such profiles that were generated in the routine diagnostic process were gathered. Raw EPIC profiles were binarized with a cut-off of beta ≥0.6 using scripts kindly provided by the authors of nanoDx²². EPIC probes not present on the 450K array used in the reference cohort were filtered out. 500 nanopore runs were simulated at 12 sparsity levels as described above. The EPIC profiles were all submitted to the Heidelberg v11b classifier (with the exception of PMC_20 which was classified with classifier v12.5). Samples were also labelled with a ‘final diagnosis’, the result of a combination of histological assessment, imaging, CNV profiling and molecular characterization which were considered the ground truth.

### Classification of Publicly Available Nanopore Sequencing Data

Nanopore sequencing data was downloaded from GSE209865²². Of note, this dataset consists of processed sequencing data, which uses a different processing method consisting of Guppy (v3.1.5) base calling, Nanopolish methylation calling, and mapping to hg19. This dataset consists of binary methylation calls for 450K methylation sites and can thus directly be used for Sturgeon classification.

### DNA Extraction

DNA is extracted from a tumor sample using an adapted QiaAmp mini (Qiagen) protocol. Ideally, a tumor sample of roughly 5×5×5 mm is used as input material. ATL buffer (180 μl) is added to the sample and the sample is briefly ground using a pestle, then 200 μl buffer AL and 20 μl proteinase K are added and the sample is moved to a 70° C. heat block. Once heated the sample is ground with a pestle every minute to improve proteinase K accessibility. When the sample contains no more solid tissue, or after 5 min of incubation and grinding, the sample is added to a Qiashredder column (Qiagen ID: 79656), not including any solid matter if still present. The column is centrifuged at 20,000 g for 1 min. 200 μl of 96% ethanol is added to the eluate and the eluate is moved to a giaAmp column and centrifuged for 1 min at 6,000 g. The column is washed with 500 μl AW1, centrifuged at 6,000 g for 1 min, then with 500 μl AW2 at 12,000 g for 1 min. The remaining ethanol is removed in a fresh elution column, centrifuged at 12,000 g for 10 s. Sample is eluted with 25 μl of MilliQ water. Samples are quantified using a Nanodrop spectrophotometer (ThermoFisher). For the first few intraoperative cases, samples where the tumor cell purity was low were encountered, resulting in low confidence scores, or high confidence scores for control tissues. As there is very limited time between sampling and processing, the tumor cell content cannot be rigorously assessed prior to DNA isolation. Instead, after the fourth case, where possible, an approach where DNA from up to three distinct sections of the sample was isolated was implemented. Simultaneously, the pathologist assesses tumor content from these same sections and calls the DNA isolation laboratory to report with which sample to continue. This procedure only slightly delays the process (three samples are processed instead of one during the DNA isolation), and the pathologist is usually able to relay this information before DNA isolation is completed.

### Flowcell Chemistry Versions

During the collection of samples, R9 MinION flowcells (which are being discontinued) were migrated from to R10.4.1 MinION flowcells. Since Sturgeon uses a list of CpG sites and their binary methylation state as input, it was not expected nor observed that an effect on classifier performance. This was also confirmed by re-sequencing and processing five samples on an R10.4.1 MinION flowcell that were previously sequenced using an R9 PromethION flowcell. An increased throughput on R10.4.1 flowcells was observed, in the range of 1,000-1,200 reads per minute and slightly higher concordance between methylation array methylation calls and nanopore sequencing methylation call (FIG. 51).

### Library Prep

Samples are library prepped depending on whether an R9 or R10 flowcell is to be used. For R9 flowcells, the Oxford Nanopore RBK004 kit was used, using 600 ng input material and following manufacturer's instructions for other steps. For R10 flowcells, the Oxford Nanopore technologies RBK114-24 kit was used, but with an adjusted protocol: for optimal results (size distribution centred around 5 kb) 3,500 ng of input material in 50 μl is first sheared using a G-Tube (Covaris SKU: 520079), centrifuging at 7,200 RPM (6,000 g) in a fixed rotor tabletop centrifuge. Subsequently 7.5 μl of the sheared input material is used for tagmentation with 2.5 μl indexing mix. Alternatively, similar results (but with wider size distributions) were obtained using 200 ng input material for the tagmentation without an added fragmentation step. For both protocols, the AMPure purification was omitted, and after tagmentation directly proceed with adapter ligation.

### Flowcell Loading

ONT MinION sequencing initializes with a pore scan, which takes around 5 min and produces no reads. Therefore, with R9 flowcells, the sequencing is started as soon as the sample arrives in the laboratory, so that sequencing commences as soon as the library is loaded onto the flowcell. Flowcells are primed using 800 μl Flush Buffer (from the ONT flowcell priming kit) at the start of the DNA isolation, after five minutes the flowcell is flushed with 200 μl Flush Buffer and sequencing is started, at which point the software will first perform a pore scan. The DNA library is loaded as soon as it is ready. By then the pore scan has typically finished and sequencing commences. It was noted that this procedure has an adverse effect for R10 flowcells, and for these sequencing was only started after loading the library on the flowcell. The sequencing itself is slowed down by the startup phase (FIG. 51), where many unligated sequencing adapters are sequenced, and then ramps up towards higher pore activity and more informative reads per minute (usually stabilizing in the range of 800-1,200 reads per minute). 10,000-20,000 reads were typically obtained within 1 h after the sample arrives in the isolation laboratory. In some, but not all, cases this is enough for a reliable diagnosis. After 90 min 40,000-60,000 reads are typically reached. After sequencing flowcells were routinely washed using the EXP-WSH004 flowcell wash kit (Oxford Nanopore Technologies) according to the manufacturer's instructions and stored for later use.

### Methylation Calling

To call methylation from R9 chemistry nanopore data Megalodon V2.5.0 was used, which runs with Guppy V5 to perform base calling and mapping to the CHM13V2 reference genome. To call per-read-per-site methylation the Rerio CpG methylation model was used as described in 23. The methylation log likelihood ratio was converted to a probability and used a cut-off of <0.3 for unmethylated and >0.7 for methylated. For R10 chemistry, Guppy V6.4 was used and the high-accuracy CpG methylation calling model to call methylation and again used the <0.3 and >0.7 cut-offs to call methylation. Methylation calls are assigned to one of the 450K CpG sites present on the Infinium methylation array using windows centered on the CpG site targeted by each probe. Different window sizes were benchmarked and observed that for R9 chemistry (and associated methylation calling procedure), 100-bp windows provide optimal results. With R10 chemistry, smaller windows do not reduce the methylation calling accuracy much, but for simplicity a 100-bp window was used. If multiple CpG sites are present within the window, majority voting is used to convert the calls to a single call per site. When multiple reads cover the same Infinium probe site, majority voting is also used to create one methylation call. When voting results in a tie, that particular site was discarded. The methylation calling error rate was evaluated by comparing the methylation calls between nanopore sequencing and the microarray data for the same samples where both methods were available. This indicated a concordance of 85-90% between binarized array data (beta cut-off at ≥0.6) and nanopore methylation calls. The error rate is evenly distributed between false positives (calling unmethylated sites as methylated) and false negatives (calling methylated sites unmethylated).

### CNV Calling

The CNV calling approach was adapted from Kuschel et al.²² Even with several hours of sequencing, coverage is still sparse (50,000 reads equates to 1 read per 64 kb of genomic sequence). Therefore, the analysis was restricted to genomic bins spanning 2 Megabases using the QDNAseq package³⁷. The coverage was normalized using a publicly available deeply sequenced Genome In a Bottle reference sample (NA12878 release 7; https://github.com/nanopore-wgs-consortium/NA12878/blob/master/Genome.md), to correct for nanopore sequencing specific mapping biases against the CHM13V2 reference genome. To obtain a log ratio, the relative coverage (sample coverage/reference coverage) per bin was first calculated, after which the log 2 of the relative coverage over the mean relative coverage is taken. The DNAcopy³⁸ R package was applied to segment the genome into over or underrepresented regions, indicating potential CNVs.

### Live Analysis

A custom R script (available on the Sturgeon github) was developed to run parallel to the sequencing software. MinKNOW (v22.12.7) was run with disabled base calling. MinKNOW outputs fast5 files, each containing 4,000 reads. The R script checks the MinKNOW output folder for a new fast5 file, and triggers if it contains 4,000 reads. Complete fast5 files are copied to a separate working directory where they are processed using either Megalodon or Guppy depending on the chemistry version. For R9 chemistry, qCat V1.1.0 (github.com/nanoporetech/qcat) is used to identify barcodes and for R10 chemistry the Guppy built-in barcoding detection algorithm was used. Depending on user settings, either the most frequent or a user-specified barcode is selected. Per-read-per-site methylation calls (Guppy v5.0.1 and Megalodon v2.5.0) or .bam files (Guppy v6.5.2) originating from reads with the selected barcode are saved and Sturgeon is used to map the methylation calls to the 450K CpG sites and classify the sample. Sequencing and analysis are performed on an ASUS TUF A15 FA507RR-HN003 W laptop with 64 Gb RAM. For R10 sequencing experiments, Guppy v6.4.6 was used with the dna_r10.4.1_e8.2_400bps_modbases_5mc_cg_hac.cfg configuration file, with disabled Q-score filtering and minimum barcode quality set at 6. As single samples were used, demultiplexing is not strictly necessary and reads were processed with and without classified barcodes.

### Adaptive Sampling

Adaptive sampling can be performed in exclusion or enrichment mode, and can be configured either through specifying a reference genome and a .bed file or a .fasta sequence file. Additionally, buffer regions can be specified, which, determines how much flanking sequence is accepted around the CpG sites of interest. Simulations of different buffer regions were performed assuming the read length distribution of earlier sequence experiments. This indicated that 5-kb buffer regions are optimal. Together, these windows span 1.3 billion base pairs (approximately 40% of the genome). Both .bed files and .fasta files were designed for 5-kb flanking regions, merging any regions that are within 25 kb using Bedtools merge (Bedtools V2.30)³⁹. When choosing between adaptive enrichment and adaptive exclusion, adaptive enrichment was opted for, as adaptive exclusion will read up to 4,000 bp before deciding to exclude a read, whereas adaptive enrichment decides after a maximum of 400 bp. Finally, initial experiments showed higher efficiency when using a reference genome and a .bed file with regions of interest compared to using a .fasta file with target sequences. The added value was assessed of adaptive sampling by running 5 samples on MinION R10.4.1 flow cells where adaptive sampling was enabled for half of the available channels. This allowed us to make a fair comparison between adaptive and non-adaptive sequencing on the same flowcell and library. All adaptive sampling experiments were performed on an Oxford Nanopore GridION device running MinKNOW 22.12.5 using high-accuracy base calling.

### REFERENCES

1. Cohen, A. R. Brain tumors in children. N. Engl. J. Med. 386, 1922-1931 (2022).
2. Duffau, H. & Mandonnet, E. The ‘onco-functional balance’ in surgery for diffuse low grade glioma: integrating the extent of resection with quality of life. Acta Neurochir. 155, 951-957 (2013).
3. Yong, R. L. & Lonser, R. R. Surgery for glioblastoma multiforme: striking a balance. World Neurosurg. 76, 528-530 (2011).
4. Djirackor, L. et al. Intraoperative DNA methylation classification of brain tumors impacts neurosurgical strategy. Neurooncol. Adv. 3, vdab149 (2021).
5. Karremann, M. et al. Diffuse high-grade gliomas with H3 K27M mutations carry a dismal prognosis independent of tumor location. Neuro Oncol. 20, 123-131 (2018).
6. Thompson, E. M. et al. Prognostic value of medulloblastoma extent of resection after accounting for molecular subgroup: a retrospective integrated clinical and molecular analysis. Lancet Oncol. 17, 484-495 (2016).
7. Venkatramani, R. et al. Supratentorial ependymoma in children: to observe or to treat following gross total resection? Pediatr. Blood Cancer 58, 380-383 (2012).
8. Ramaswamy, V. et al. Therapeutic impact of cytoreductive surgery and irradiation of posterior fossa ependymoma in the molecular era: a retrospective multicohort analysis. J. Clin. Oncol. 34, 2468-2477 (2016).
9. Pajtler, K. W. et al. The current consensus on the clinical management of intracranial ependymoma and its distinct molecular variants. Acta Neuropathol. 133, 5-12 (2017).
10. Egiz, A., Kannas, S. & Asl, S. F. The impact of surgical resection and adjuvant therapy on survival in pediatric patients with atypical teratoid/rhabdoid tumor: systematic review and pooled survival analysis. World Neurosurg. 164, 216-227 (2022).
11. Drexler, R. et al. DNA methylation subclasses predict the benefit from gross total tumor resection in IDH-wildtype glioblastoma patients. Neuro. Oncol. 25, 315-325 (2023).
12. Wijnenga, M. M. J. et al. The impact of surgery in molecularly defined low-grade glioma: an integrated clinical, radiological, and molecular analysis. Neuro. Oncol. 20, 103-112 (2018).
13. Papanicolau-Sengos, A. & Aldape, K. DNA methylation profiling: an emerging paradigm for cancer diagnosis. Annu. Rev. Pathol. 17, 295-321 (2022).
14. Capper, D. et al. DNA methylation-based classification of central nervous system tumours. Nature 555, 469-474 (2018).
15. Jaunmuktane, Z. et al. Methylation array profiling of adult brain tumours: diagnostic outcomes in a large, single centre. Acta Neuropathol Commun 7, 24 (2019).
16. Priesterbach-Ackley, L. P. et al. Brain tumour diagnostics using a DNA methylation-based classifier as a diagnostic support tool. Neuropathol. Appl. Neurobiol. 46, 478-492 (2020).
17. Sandoval, J. et al. Validation of a DNA methylation microarray for 450,000 CpG sites in the human genome. Epigenetics 6, 692-702 (2011).
18. Moran, S., Arribas, C. & Esteller, M. Validation of a DNA methylation microarray for 850,000 CpG sites of the human genome enriched in enhancer sequences. Epigenomics 8, 389-399 (2016).
19. Gorzynski, J. E. et al. Ultrarapid nanopore genome sequencing in a critical care setting. N. Engl. J. Med. 386, 700-702 (2022).
20. Sagniez, M. et al. Real-time molecular classification of leukemias. Preprint at medRxiv https://doi.org/10.1101/2022.06.22.22276550 (2022).
21. Xu, L. & Seki, M. Recent advances in the detection of base modifications using the Nanopore sequencer. J. Hum. Genet. 65, 25-33 (2020).
22. Kuschel, L. P. et al. Robust methylation-based classification of brain tumors using nanopore sequencing. Preprint at bioRxiv https://doi.org/10.1101/2021.03.06.21252627 (2021).
23. Yuen, Z. W.-S. et al. Systematic benchmarking of tools for CpG methylation detection from nanopore sequencing. Nat. Commun. 12, 3438 (2021).
24. Guo, C., Pleiss, G., Sun, Y. & Weinberger, K. Q. On calibration of modern neural networks. In Proc. 34^{th} International Conference on Machine Learning, Vol. 70 (eds. Precup, D. & Teh, Y. W.) 1321-1330 (Proceedings of Machine Learning Research, 2017).
25. Capper, D. Practical implementation of DNA methylation and copy-number-based CNS tumor diagnostics: the Heidelberg experience. Acta Neuropathol. 136, 181-210 (2018).
26. Verburg, N. et al. Spatial concordance of DNA methylation classification in diffuse glioma. Neuro. Oncol. 23, 2054-2065 (2021).
27. Euskirchen, P. et al. Same-day genomic and epigenomic diagnosis of brain tumors using real-time nanopore sequencing. Acta Neuropathol. 134, 691-703 (2017).
28. Molinaro, A. M. et al. Association of maximal extent of resection of contrast-enhanced and non-contrast-enhanced tumor with survival within molecular subgroups of patients with newly diagnosed glioblastoma. JAMA Oncol. 6, 495-503 (2020).
29. Cahill, D. P. Extent of resection of glioblastoma: a critical evaluation in the molecular era. Neurosurg. Clin. N. Am. 32, 23-29 (2021).
30. Stummer, W. et al. Fluorescence-guided surgery with 5-aminolevulinic acid for resection of malignant glioma: a randomised controlled multicentre phase III trial. Lancet Oncol. 7, 392-401 (2006).
31. Loose, M., Malla, S. & Stout, M. Real-time selective sequencing using nanopore technology. Nat. Methods 13, 751-754 (2016).
32. WHO Classification of Tumours Editorial Board. Central Nervous System Tumours (International Agency for Research on Cancer, 2022).
33. Rieke, N. et al. The future of digital health with federated learning. npj Digit. Med. 3, 119 (2020).
34. Bregy, A. et al. The role of Gliadel wafers in the treatment of high-grade gliomas. Expert Rev. Anticancer Ther. 13, 1453-1461 (2013).
35. Mathew, E. N., Berry, B. C., Yang, H. W., Carroll, R. S. & Johnson, M. D. Delivering therapeutics to glioblastoma: overcoming biological constraints. Int. J. Mol. Sci. 23, 1711 (2022).
36. Loshchilov, I. AdamW-and-SGDW: Decoupled Weight Decay Regularization (ICLR 2019). (Github).
37. Scheinin, I. et al. DNA copy number analysis of fresh and formalin-fixed specimens by shallow whole-genome sequencing with identification and exclusion of problematic regions in the genome assembly. Genome Res. 24, 2022-2032 (2014).
38. Seshan, V. E. & Olshen, A. DNAcopy: DNA copy number data analysis. R package version.
39. Quinlan, A. R. & Hall, I. M. BEDTools: a flexible suite of utilities for comparing genomic features. Bioinformatics 26, 841-842 (2010).

### Additional Implementation Detail

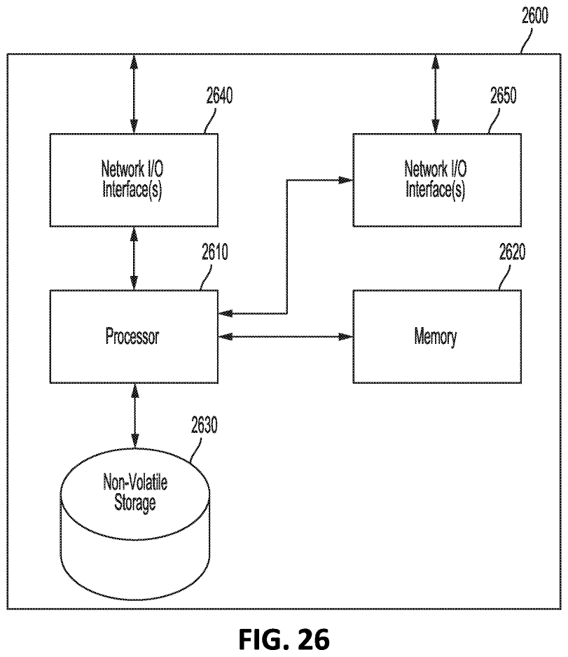

An illustrative implementation of a computer system 2600 that may be used in connection with any of the embodiments of the technology described herein is shown in FIG. 26. The computer system 2600 includes one or more processors 2610 and one or more articles of manufacture that comprise non-transitory computer-readable storage media (e.g., memory 2620 and one or more non-volatile storage media 2630). The processor 2610 may control writing data to and reading data from the memory 2620 and the non-volatile storage device 2630 in any suitable manner, as the aspects of the technology described herein are not limited to any particular techniques for writing or reading data. To perform any of the functionality described herein, the processor 2610 may execute one or more processor-executable instructions stored in one or more non-transitory computer-readable storage media (e.g., the memory 2620), which may serve as non-transitory computer-readable storage media storing processor-executable instructions for execution by the processor 2610.

Computing device 2600 may also include a network input/output (I/O) interface 2640 via which the computing device may communicate with other computing devices (e.g., over a network), and may also include one or more user I/O interfaces 2650, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats. Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### CLAUSES

1. A computer-implemented method for identifying a DNA modification, preferably DNA methylation, in DNA sequencing data, for the intraoperative classification of a disease and/or disorder, preferably a tumor, more preferably a brain tumor, of a subject on which the operation is performed, the method comprising the steps of:
   a) obtaining DNA sequencing data of the subject, preferably wherein the DNA sequencing data is obtained by using a sequencing method capable of directly sensing a DNA modification, more preferably nanopore sequencing;
   b) identifying the DNA modification status of the DNA sequencing data, thereby obtaining a DNA modification profile;
   c) classifying the DNA modification profile using a DNA modification based deep learning (DL)-classifier thereby obtaining a classification score for a disease and/or disorder;
   wherein the DNA modification based DL-classifier is trained with a training set, wherein the training is performed in a pre-operative training routine; and
   wherein the pre-operative training routine comprises training the DNA modification based DL-classifier on a training set generated from reference data comprising DNA modification data, preferably wherein the DNA modification data is any one or more selected from the group of microarray data, preferably hybridization microarray data, whole genome bisulfite sequencing data, PacBio SMRT sequencing data, TAPS-sequencing data or nanopore sequencing data.
2. A computer-implemented method for identifying a DNA modification, preferably DNA methylation, of a sample of a subject, said sample comprising DNA for the intraoperative classification of a disease and/or disorder, preferably a tumor, more preferably a brain tumor, of a subject on which the operation is performed, the method comprising the steps of:
   a) providing the sample comprising DNA;
   b) sequencing the DNA of the sample, preferably by using a sequencing method capable of directly sensing a DNA modification, more preferably nanopore sequencing, thereby obtaining DNA sequencing data;
   c) identifying the DNA modification status of the DNA sequencing data, thereby obtaining a DNA modification profile;
   d) classifying the DNA modification profile using a DNA modification based deep learning (DL)-classifier thereby obtaining a classification score for a disease and/or disorder;
   wherein the DNA modification based DL-classifier is trained with a training set, wherein the training is performed in a pre-operative training routine; and
   wherein the pre-operative training routine comprises training the DNA modification based DL-classifier on a training set from reference data comprising DNA modification data, preferably wherein the DNA modification data is any one or more selected from the group of microarray data, preferably hybridization microarray data, whole genome sequencing data, whole genome sequencing bisulfite sequencing, PacBio SMRT sequencing data, TAPS-sequencing data or nanopore sequencing data.
3. The computer-implemented method according to any one of the previous clauses, wherein the reference data comprising DNA modification data is obtained by using a technique that differs from the technique used for obtaining DNA sequencing data of the subject and/or of the sample, preferably wherein said sample is obtained intraoperatively.
4. The computer-implemented method according to any one of the previous clauses, wherein the pre-operative training routine comprises:
a) providing reference data comprising DNA modification data, preferably whole genome sequencing data, as input in a simulation module, preferably wherein the DNA modification data comprises a DNA modification profile associated with a cell type and/or cell state of interest;
b) using the simulation module to generate a training set from the reference data, wherein the training set comprises data having a DNA modification profile, preferably a nanopore profile; and
c) training the DNA modification based DL-classifier on the training set.
5. The computer-implemented method according to any one of the previous clauses, wherein generating the training set from the reference data comprises any one or more of:
   Binarization;
   Non-uniform subsampling; and
   Error simulation.
preferably, wherein non-uniform subsampling comprises the random selection of CpG sites and the extension of the size of the sequencing reads and/or the sequencing time, and/or, preferably wherein error simulation comprises the generating of an error-rate of at least 10% in DNA modification-calling.
6. The computer-implemented method according to clause 4, wherein the pre-operative training routine further comprises after c) the step d), wherein step d) comprises an intraoperative validation routine comprising validation of the DNA modification based DL-classifier on whole genome sequencing data, preferably nanopore sequencing data.
7. The computer-implemented method according to any one of the previous clauses, wherein the pre-operative training routine comprises down sampling of the DNA modification data, preferably whole genome sequencing data and/or wherein the classifying of the DNA modification profile comprises up sampling of the whole genome sequencing data, preferably nanopore sequencing data.
8. The computer-implemented method according to any one of the previous clauses, wherein the DNA modification classification provides a diagnosis of a tumor species, preferably of a brain tumor species.
9. The computer-implemented method according to any one of the previous clauses, wherein the DNA sequencing data is obtained by whole genome sequencing, preferably by nanopore sequencing.
10. The computer-implemented method according to any one of the previous clauses, wherein the sample comprising DNA is a sample, preferably a tumor sample, and wherein the sample is derived from a subject intraoperatively; and/or wherein the subject is a human subject, preferably a human subject having a disease and/or disorder, preferably a tumor, more preferably a brain tumor, and preferably wherein the subject is under surgery.
11. The computer-implemented method according to any one of the previous clauses, wherein the DNA modification data comprises a DNA modification selected from the group consisting of: methylation or oxidation.
12. The computer-implemented method according to clause 11, wherein methylation comprises any one or more of CpG methylation, GpC methylation, 4mC methylation, 6 mA methylation, 5mC methylation, 5-hydroxymethylation, preferably 5mC methylation.
13. A method for identifying a disease and/or disorder, preferably a tumor, more preferably a brain tumor, comprising performing the computer-implemented invention according to any one of the previous clauses.
14. A method of the pre-operative configuring of a DNA modification based DL-classifier, preferably a DNA modification based DL-classifier as used in any one of the previous clauses, to receive a sample comprising DNA and/or to receive DNA sequencing data and to generate a classification score for a disease and/or disorder, preferably for a tumor species, the method comprising: training a DNA modification based DL-classifier, which runs on a processor coupled to memory, comprising a pre-operative training routine using as input reference data, wherein the reference data comprises DNA modification data; wherein the DNA modification based DL-classifier after training is configured to receive a DNA modification profile; and wherein the DNA modification based DL-classifier generates a classification score output that indicates whether the DNA modification of the DNA modification profile is indicative for a disease and/or disorder.
15. The method of the pre-operative configuring of a DNA modification based DL-classifier according to clause 14, wherein the DNA modification data comprises any one or more selected from the group of microarray data, preferably hybridization microarray data, whole genome bisulfite sequencing data, TAPS-sequencing data or nanopore sequencing data.
16. The method of the pre-operative configuring of a DNA modification based DL-classifier according to clause 14 or 15, wherein the DNA sequencing data is obtained by using a sequencing method capable of directly sensing a DNA modification, more preferably nanopore sequencing.
17. The method of the pre-operative configuring of a DNA modification based DL-classifier according to any one of the previous clauses, wherein the reference data comprising DNA modification data is obtained by using a technique that differs from the technique used for obtaining DNA sequencing data of the subject and/or of the sample, preferably wherein said sample is obtained intraoperatively.
18. The computer-implemented method according to any one of the previous clauses or method according to any one of the previous clauses, wherein the disease and/or disorder is a cancer, preferably a tumor, preferably a brain tumor.
19. A method of treatment comprising the step of performing the computer-implemented method according to any one of the previous clauses.
20. A method of treatment comprising the steps of:
performing the computer-implemented method according to any one of the previous clauses; and
treating a tumor, preferably a brain tumor.
21. A method of surgery comprising the step of performing the computer-implemented method according to any one of the previous clauses.
22. A method of surgery comprising the steps of:
   performing the computer-implemented method according to any one of the previous clauses; and
   removing a tumor, preferably a brain tumor by surgery, preferably by surgical resection.
23. A method for identifying a type of tumor in a patient based on DNA methylation status of a subset of a plurality of candidate DNA methylation sites, the method comprising:
   using at least one computer hardware processor to perform:
      (A) obtaining DNA sequencing data previously obtained in part by sequencing a biological sample obtained from the patient;
      (B) identifying, using the DNA sequencing data, the subset of the plurality of candidate DNA methylation sites as those DNA methylation sites for which the DNA sequencing data indicates DNA methylation status;
      (C) generating a sparse DNA methylation profile for the patient using the DNA sequencing data, the sparse DNA methylation profile indicating DNA methylation status of sites in the identified subset of the plurality of candidate DNA methylation sites; and
      (D) identifying the type of tumor in the patient by processing the sparse DNA methylation profile using a trained neural network model to obtain output indicative of the type of the tumor in the patient.
24. The method of clause 23, further comprising:
   prior to performing (A), sequencing the biological sample obtained from the patient to obtain raw sequencing data and performing base calling on the raw sequencing data to obtain the DNA sequencing data.
25. The method of clause 24, wherein sequencing the biological sample comprises sequencing the biological sample using nanopore sequencing.
26. The method of clause 25, wherein sequencing the biological sample consists of sequencing the biological sample for an amount of time between 10 and 45 minutes to obtain the DNA sequencing data.
27. The method of clause 23, further comprising:
   while the patient is undergoing surgery:
      obtaining the biological sample from the patient;
      sequencing the biological sample using nanopore sequencing to generate the DNA sequencing data; and
      performing (A), (B), (C), and (D).
28. The method of clause 27, wherein the output indicative of the type of tumor in the patient includes a confidence associated with the type of tumor indicated, the method further comprising:
   while the patient is undergoing the surgery and when the confidence is below a predetermined threshold confidence:
      continuing to sequence the biological sample using the nanopore sequencing to generate additional DNA sequencing data;
      augmenting the sparse DNA methylation profile using information in the additional DNA sequencing data to obtain an augmented sparse DNA methylation profile; and
      identifying the type of tumor in the patient by processing the augmented sparse DNA methylation profile using the trained neural network model to obtain a second output indicative of the type of the tumor in the patient.
29. The method of clause 27, further comprising:
   stopping the surgery based on the output indicative of the type of tumor in the patient; or
   performing the surgery in a manner that depends on the output indicative of the type of the tumor in the patient.
30. The method of clause 23, wherein the tumor is a central nervous system (CNS) tumor.
31. The method of clause 23, wherein:
   the DNA sequencing data indicates DNA methylation status only for sites in the identified subset of plurality of candidate DNA methylation sites, and
   the identified subset consists of between 0.001% and 4.0% of sites in the plurality of candidate DNA methylation sites.
32. The method of clause 31, wherein the plurality of candidate DNA methylation sites consists of between 400,000 and 500,000 sites or between 800,000 and 900,000 sites.
33. The method of clause 31, wherein:
   the DNA sequencing data was obtained by nanopore sequencing; and
   the plurality of candidate DNA methylation sites consists of a number of sites substantially equal to a number of CpG probes in a methylation profiling microarray.
34. The method of clause 23, wherein generating the sparse DNA methylation profile comprises:
   generating a data structure representing the methylation profile, the data structure configured to store values for a plurality of entries corresponding to the plurality of candidate DNA methylation sites; and
   setting, based on the DNA sequencing data, values for a subset of the plurality of entries that correspond to the identified subset of the plurality of candidate DNA methylation sites,
   wherein a first value for a first entry in the subset of the plurality of entries indicates presence or absence of DNA methylation at a candidate DNA methylation site in the identified subset to which the first entry corresponds.
35. The method of clause 34, wherein values of entries, which are in the plurality of entries but not in the subset of the plurality of entries, indicate that DNA methylation status was not indicated by the DNA sequencing data for candidate DNA methylation sites to which the entries correspond.
36. The method of clause 35, wherein processing the sparse DNA methylation profile using the trained neural network model comprises processing values stored in the data structure using the trained neural network model.
37. The method of clause 35, wherein the subset of the plurality of entries in the data structure consists of between 0.001 and 4% of all entries in the data structure.
38. The method of clause 35,
   wherein the data structure represents a vector,
   wherein, within the vector, each value for a particular entry in the subset of the plurality of entries that corresponds to the identified subset of the plurality of candidate DNA methylation sites is either a 1, indicating presence of a DNA methylation, or −1, which indicates absence of a DNA methylation at the candidate DNA methylation site to which the particular entry corresponds, and
   wherein, within the vector, each value for a particular entry not in the subset of the plurality of entries that corresponds to the identified subset of the plurality of candidate DNA methylation sites, is set to 0 indicating that the DNA sequencing data provides no indication as to the DNA methylation status at the candidate DNA methylation site to which the particular entry corresponds.
39. The method of clause 23, wherein the trained neural network comprises a plurality of fully connected layers with non-linear activations therebetween and a classification head.
40. The method of clause 23, wherein the trained neural network comprises:
   a first fully connected layer having a first input size corresponding to a total number of sites in the plurality of candidate DNA methylation sites and a first output size smaller than the input size;
   a second fully connected layer having a second input size equal to the first output size and a second output size smaller than the second input size;
   a third fully connected layer having a third input size equal to the second output size and a third output size smaller than the third input size and corresponding to a number of candidate types of the tumor; and
   a first non-linear activation between the first and second fully connected layers and a second non-linear activation between the second and third fully connected layers.
41. The method of clause 23,
   wherein the trained neural network comprises at least 10 million parameters, and
   wherein processing the sparse DNA methylation profile using the trained neural network comprises determining the output indicative of the type of the tumor in the patient by using values in the sparse DNA methylation profile and values of the at least 10 million parameters.
42. The method of clause 23, wherein the output indicative of the type of tumor in the patient comprises a plurality of likelihoods corresponding to a respective plurality of tumor types, wherein each particular likelihood of the plurality of likelihoods indicates a likelihood that the patient has a respective particular type of tumor in the plurality of tumor types.
43. The method of clause 25, wherein sequencing the biological sample using nanopore sequencing comprises sequencing the biological sample using nanopore sequence with adaptive sampling.
44. The method of clause 25, wherein sequencing the biological sample using nanopore sequencing comprising:
   while a DNA strand is being sequenced using a nanopore part of a nanopore sequencing apparatus:
      obtaining a partial sequence of the DNA strand using measurements generated by the nanopore;
      determining whether the partial sequence maps to at least one of the plurality of candidate DNA methylation sites;
      when it is determined that the partial sequence does not map to at least one of the plurality of candidate DNA methylation sites, ejecting the DNA strand from the nanopore; and
      when it is determined that the partial sequence maps to the at least one of the plurality of candidate DNA methylation sites, continuing to sequence the DNA strand.
45. The method of clause 23, further comprising:
   generating training data using microarray methylation data; and
   training a neural network model using the training data to obtain the trained neural network model.
46. The method of clause 45, wherein generating the training data using microarray methylation data comprises generating, from microarray methylation data, sparse DNA methylation profiles representative of types of sparse DNA methylation profiles that would be generated using nanopore sequencing with a threshold amount of time.
47. A system, comprising:
   at least one computer hardware processor; and
   at least one non-transitory computer readable storage medium storing processor-executable instructions that, when executed by the at least one computer hardware processor, cause the at least one computer hardware processor to perform a method of any one of clauses 23-46.
48. At least one non-transitory computer readable storage medium storing processor-executable instructions that, when executed by at least one computer hardware processor, cause the at least one computer hardware processor to perform a method of any one of clauses 23-46.
49. A system for identifying a type of tumor in a patient based on DNA methylation status of a subset of a plurality of candidate DNA methylation sites, the system comprising:
   a nanopore sequencing apparatus; and
   at least one computing device configured to perform:
      (A) obtaining DNA sequencing data at least in part by sequencing a biological sample obtained from the patient using the nanopore sequencing apparatus;
      (B) identifying, using the DNA sequencing data, the subset of the plurality of candidate DNA methylation sites as those DNA methylation sites for which the DNA sequencing data indicates DNA methylation status;
      (C) generating a sparse DNA methylation profile for the patient using the DNA sequencing data, the sparse DNA methylation profile indicating DNA methylation status of sites in the identified subset of the plurality of candidate DNA methylation sites; and
      (D) identifying the type of tumor in the patient by processing the sparse DNA methylation profile using a trained neural network model to obtain output indicative of the type of the tumor in the patient.

1-49. (canceled)

50. A computer-implemented method for identifying a DNA modification, preferably DNA methylation, in DNA sequencing data, for the intraoperative classification of a disease and/or disorder, preferably a tumor, more preferably a brain tumor, of a subject on which the operation is performed, the method comprising the steps of:
a) obtaining DNA sequencing data of the subject, preferably wherein the DNA sequencing data is obtained by using a sequencing method capable of directly sensing a DNA modification, more preferably nanopore sequencing, even more preferably wherein the DNA sequencing data is obtained by whole genome sequencing
b) identifying the DNA modification status of the DNA sequencing data, thereby obtaining a DNA modification profile;
c) classifying the DNA modification profile using a DNA modification based deep learning (DL)-classifier thereby obtaining a classification score for a disease and/or disorder;
wherein the DNA modification based DL-classifier is trained with a training set, wherein the training is performed in a pre-operative training routine; and
wherein the pre-operative training routine comprises training the DNA modification based DL-classifier on a training set generated from reference data comprising DNA modification data, preferably wherein the DNA modification data is any one or more selected from the group of microarray data, preferably hybridization microarray data, whole genome sequencing data, whole genome bisulfite sequencing data, PacBio SMRT sequencing data, TAPS-sequencing data or nanopore sequencing data.

51. The computer-implemented method according to claim 50,
wherein the pre-operative training routine comprises:
a) providing reference data comprising DNA modification data, preferably whole genome sequencing data, as input in a simulation module, preferably wherein the DNA modification data comprises a DNA modification profile associated with a cell type and/or cell state of interest;
b) using the simulation module to generate a training set from the reference data, wherein the training set comprises data having a DNA modification profile, preferably a nanopore profile; and
c) training the DNA modification based DL-classifier on the training set, preferably wherein the pre-operative training routine further comprises after c) the step d), wherein step d) comprises an intraoperative validation routine comprising validation of the DNA modification based DL-classifier on whole genome sequencing data, preferably nanopore sequencing data.

52. The computer-implemented method according to claim 50,
wherein generating the training set from the reference data comprises any one or more of:
Binarization;
Non-uniform subsampling; and
Error simulation,
preferably, wherein non-uniform subsampling comprises the random selection of CpG sites and the extension of the size of the sequencing reads and/or the sequencing time, and/or, preferably wherein error simulation comprises the generating of an error-rate of at least 10% in DNA modification-calling.

53. The computer-implemented method according to claim 50,
wherein the pre-operative training routine comprises down sampling of the DNA modification data, preferably whole genome sequencing data and/or wherein the classifying of the DNA modification profile comprises up sampling of the whole genome sequencing data, preferably nanopore sequencing data, preferably wherein the DNA modification classification provides a diagnosis of a tumor species, preferably of a brain tumor species.

54. A method of the pre-operative configuring of a DNA modification based DL-classifier, preferably a DNA modification based DL-classifier as used in claim 50, to receive a sample comprising DNA and/or to receive DNA sequencing data and to generate a classification score for a disease and/or disorder, preferably for a tumor species, the method comprising: training a DNA modification based DL-classifier, which runs on a processor coupled to memory, comprising a pre-operative training routine using as input reference data, wherein the reference data comprises DNA modification data; wherein the DNA modification based DL-classifier after training is configured to receive a DNA modification profile; and wherein the DNA modification based DL-classifier generates a classification score output that indicates whether the DNA modification of the DNA modification profile is indicative for a disease and/or disorder, preferably wherein the DNA modification data comprises any one or more selected from the group of microarray data, preferably hybridization microarray data, whole genome bisulfite sequencing data, TAPS-sequencing data or nanopore sequencing data.

55. The method of the pre-operative configuring of a DNA modification based DL-classifier according to claim 54, wherein the DNA sequencing data is obtained by using a sequencing method capable of directly sensing a DNA modification, more preferably nanopore sequencing, further preferably wherein the reference data comprising DNA modification data is obtained by using a technique that differs from the technique used for obtaining DNA sequencing data of the subject and/or of the sample, preferably wherein said sample is obtained intraoperatively.

56. The computer-implemented method according to claim 50, wherein the disease and/or disorder is a cancer, preferably a tumor, preferably a brain tumor.

57. A computer-implemented method for identifying a DNA modification, preferably DNA methylation, of a sample of a subject, said sample comprising DNA for the intraoperative classification of a disease and/or disorder, preferably a tumor, more preferably a brain tumor, of a subject on which the operation is performed, the method comprising the steps of:
a) providing the sample comprising DNA;
b) sequencing the DNA of the sample, preferably by using a sequencing method capable of directly sensing a DNA modification, more preferably nanopore sequencing, thereby obtaining DNA sequencing data even more preferably wherein the DNA sequencing data is obtained by whole genome sequencing;
c) identifying the DNA modification status of the DNA sequencing data, thereby obtaining a DNA modification profile;
d) classifying the DNA modification profile using a DNA modification based deep learning (DL)-classifier thereby obtaining a classification score for a disease and/or disorder;
wherein the DNA modification based DL-classifier is trained with a training set, wherein the training is performed in a pre-operative training routine; and
wherein the pre-operative training routine comprises training the DNA modification based DL-classifier on a training set generated from reference data comprising DNA modification data, preferably wherein the DNA modification data is any one or more selected from the group of microarray data, preferably hybridization microarray data, whole genome sequencing data, whole genome sequencing bisulfite sequencing, PacBio SMRT sequencing data, TAPS-sequencing data or nanopore sequencing data, preferably wherein the sample comprising DNA is a sample, preferably a tumor sample, and wherein the sample is derived from a subject intraoperatively; and/or wherein the subject is a human subject, preferably a human subject having a disease and/or disorder, preferably a tumor, more preferably a brain tumor, and preferably wherein the subject is under surgery, preferably wherein methylation comprises any one or more of CpG methylation, GpC methylation, 4mC methylation, 6 mA methylation, 5mC methylation, 5-hydroxymethylation, preferably 5mC methylation.

58. The computer-implemented method according to claim 57, wherein the reference data comprising DNA modification data is obtained by using a technique that differs from the technique used for obtaining DNA sequencing data, preferably wherein the DNA modification data comprises a DNA modification selected from the group consisting of: methylation or oxidation.

59. The computer-implemented method according to claim 57,
wherein the pre-operative training routine comprises:
c) providing reference data comprising DNA modification data, preferably whole genome sequencing data, as input in a simulation module, preferably wherein the DNA modification data comprises a DNA modification profile associated with a cell type and/or cell state of interest;
d) using the simulation module to generate a training set from the reference data, wherein the training set comprises data having a DNA modification profile, preferably a nanopore profile; and
c) training the DNA modification based DL-classifier on the training set, preferably wherein the pre-operative training routine further comprises after c) the step d), wherein step d) comprises an intraoperative validation routine comprising validation of the DNA modification based DL-classifier on whole genome sequencing data, preferably nanopore sequencing data.

60. The computer-implemented method according to claim 57,
wherein generating the training set from the reference data comprises any one or more of:
Binarization;
Non-uniform subsampling; and
Error simulation,
preferably, wherein non-uniform subsampling comprises the random selection of CpG sites and the extension of the size of the sequencing reads and/or the sequencing time, and/or, preferably wherein error simulation comprises the generating of an error-rate of at least 10% in DNA modification-calling.

61. The computer-implemented method according to claim 57,
wherein the pre-operative training routine comprises down sampling of the DNA modification data, preferably whole genome sequencing data and/or wherein the classifying of the DNA modification profile comprises up sampling of the whole genome sequencing data, preferably nanopore sequencing data, preferably wherein the DNA modification classification provides a diagnosis of a tumor species, preferably of a brain tumor species.

62. A method of the pre-operative configuring of a DNA modification based DL-classifier, preferably a DNA modification based DL-classifier as used in claim 57, to receive a sample comprising DNA and/or to receive DNA sequencing data and to generate a classification score for a disease and/or disorder, preferably for a tumor species, the method comprising: training a DNA modification based DL-classifier, which runs on a processor coupled to memory, comprising a pre-operative training routine using as input reference data, wherein the reference data comprises DNA modification data; wherein the DNA modification based DL-classifier after training is configured to receive a DNA modification profile; and wherein the DNA modification based DL-classifier generates a classification score output that indicates whether the DNA modification of the DNA modification profile is indicative for a disease and/or disorder, preferably wherein the DNA modification data comprises any one or more selected from the group of microarray data, preferably hybridization microarray data, whole genome bisulfite sequencing data, TAPS-sequencing data or nanopore sequencing data,
wherein the DNA sequencing data is obtained by using a sequencing method capable of directly sensing a DNA modification, more preferably nanopore sequencing, further preferably wherein the reference data comprising DNA modification data is obtained by using a technique that differs from the technique used for obtaining DNA sequencing data of the subject and/or of the sample, preferably wherein said sample is obtained intraoperatively.

63. The computer-implemented method according to claim 57, wherein the disease and/or disorder is a cancer, preferably a tumor, preferably a brain tumor.

64. A method for identifying a type of tumor in a patient based on DNA methylation status of a subset of a plurality of candidate DNA methylation sites, preferably wherein the tumor is a central nervous system (CNS) tumor, the method comprising:
using at least one computer hardware processor to perform:
(A) obtaining DNA sequencing data previously obtained in part by sequencing a biological sample obtained from the patient;
(B) identifying, using the DNA sequencing data, the subset of the plurality of candidate DNA methylation sites as those DNA methylation sites for which the DNA sequencing data indicates DNA methylation status, preferably wherein:
the DNA sequencing data indicates DNA methylation status only for sites in the identified subset of plurality of candidate DNA methylation sites, and
the identified subset consists of between 0.001% and 4.0% of sites in the plurality of candidate DNA methylation sites;
(C) generating a sparse DNA methylation profile for the patient using the DNA sequencing data, the sparse DNA methylation profile indicating DNA methylation status of sites in the identified subset of the plurality of candidate DNA methylation sites; and
(D) identifying the type of tumor in the patient by processing the sparse DNA methylation profile using a trained neural network model to obtain output indicative of the type of the tumor in the patient, preferably further comprising:
prior to performing (A), sequencing the biological sample obtained from the patient to obtain raw sequencing data and performing base calling on the raw sequencing data to obtain the DNA sequencing data, preferably wherein sequencing the biological sample comprises sequencing the biological sample using nanopore sequencing, more preferably wherein sequencing the biological sample consists of sequencing the biological sample for an amount of time between 10 and 45 minutes to obtain the DNA sequencing data.

65. The method of claim 64, further comprising:
while the patient is undergoing surgery:
obtaining the biological sample from the patient;
sequencing the biological sample using nanopore sequencing to generate the DNA sequencing data; and
performing (A), (B), (C), and (D), preferably wherein the output indicative of the type of tumor in the patient includes a confidence associated with the type of tumor indicated, the method further comprising:
while the patient is undergoing the surgery and when the confidence is below a predetermined threshold confidence:
continuing to sequence the biological sample using the nanopore sequencing to generate additional DNA sequencing data;
augmenting the sparse DNA methylation profile using information in the additional DNA sequencing data to obtain an augmented sparse DNA methylation profile; and
identifying the type of tumor in the patient by processing the augmented sparse DNA methylation profile using the trained neural network model to obtain a second output indicative of the type of the tumor in the patient, more preferably further comprising:
stopping the surgery based on the output indicative of the type of tumor in the patient; or
performing the surgery in a manner that depends on the output indicative of the type of the tumor in the patient, preferably wherein the plurality of candidate DNA methylation sites consists of between 400,000 and 500,000 sites or between 800,000 and 900,000 sites, more preferably wherein:
the DNA sequencing data was obtained by nanopore sequencing; and
the plurality of candidate DNA methylation sites consists of a number of sites substantially equal to a number of CpG probes in a methylation profiling microarray.

66. The method of claim 64, wherein generating the sparse DNA methylation profile comprises:
generating a data structure representing the methylation profile, the data structure configured to store values for a plurality of entries corresponding to the plurality of candidate DNA methylation sites; and
setting, based on the DNA sequencing data, values for a subset of the plurality of entries that correspond to the identified subset of the plurality of candidate DNA methylation sites,
wherein a first value for a first entry in the subset of the plurality of entries indicates presence or absence of DNA methylation at a candidate DNA methylation site in the identified subset to which the first entry corresponds, preferably wherein values of entries, which are in the plurality of entries but not in the subset of the plurality of entries, indicate that DNA methylation status was not indicated by the DNA sequencing data for candidate DNA methylation sites to which the entries correspond, more preferably wherein processing the sparse DNA methylation profile using the trained neural network model comprises processing values stored in the data structure using the trained neural network model, and/or wherein the subset of the plurality of entries in the data structure consists of between 0.001 and 4% of all entries in the data structure; and/or
wherein the data structure represents a vector,
wherein, within the vector, each value for a particular entry in the subset of the plurality of entries that corresponds to the identified subset of the plurality of candidate DNA methylation sites is either a 1, indicating presence of a DNA methylation, or −1, which indicates absence of a DNA methylation at the candidate DNA methylation site to which the particular entry corresponds, and
wherein, within the vector, each value for a particular entry not in the subset of the plurality of entries that corresponds to the identified subset of the plurality of candidate DNA methylation sites, is set to 0 indicating that the DNA sequencing data provides no indication as to the DNA methylation status at the candidate DNA methylation site to which the particular entry corresponds.

67. The method of claim 64, wherein the trained neural network comprises a plurality of fully connected layers with non-linear activations therebetween and a classification head and/or wherein the trained neural network comprises:
a first fully connected layer having a first input size corresponding to a total number of sites in the plurality of candidate DNA methylation sites and a first output size smaller than the input size;
a second fully connected layer having a second input size equal to the first output size and a second output size smaller than the second input size;
a third fully connected layer having a third input size equal to the second output size and a third output size smaller than the third input size and corresponding to a number of candidate types of the tumor; and
a first non-linear activation between the first and second fully connected layers and a second non-linear activation between the second and third fully connected layers;
preferably wherein the trained neural network comprises at least 10 million parameters, and
wherein processing the sparse DNA methylation profile using the trained neural network comprises determining the output indicative of the type of the tumor in the patient by using values in the sparse DNA methylation profile and values of the at least 10 million parameters; and/or
wherein the output indicative of the type of tumor in the patient comprises a plurality of likelihoods corresponding to a respective plurality of tumor types, wherein each particular likelihood of the plurality of likelihoods indicates a likelihood that the patient has a respective particular type of tumor in the plurality of tumor types.

68. The method of claim 64, wherein sequencing the biological sample using nanopore sequencing comprises sequencing the biological sample using nanopore sequence with adaptive sampling and/or wherein sequencing the biological sample using nanopore sequencing comprising:
while a DNA strand is being sequenced using a nanopore part of a nanopore sequencing apparatus:
obtaining a partial sequence of the DNA strand using measurements generated by the nanopore;
determining whether the partial sequence maps to at least one of the plurality of candidate DNA methylation sites;
when it is determined that the partial sequence does not map to at least one of the plurality of candidate DNA methylation sites, ejecting the DNA strand from the nanopore; and
when it is determined that the partial sequence maps to the at least one of the plurality of candidate DNA methylation sites, continuing to sequence the DNA strand.

69. The method of claim 64, further comprising:
generating training data using microarray methylation data; and
training a neural network model using the training data to obtain the trained neural network model, preferably wherein generating the training data using microarray methylation data comprises generating, from microarray methylation data, sparse DNA methylation profiles representative of types of sparse DNA methylation profiles that would be generated using nanopore sequencing with a threshold amount of time.
